(19) United States
(12) Patent Application Publication
(10) Pub. No.: US 2011/0177600 A1
(43) Pub. Date: Jul. 21, 2011

(54) PROTEIN PRODUCTION USING EUKARYOTIC CELL LINES

(76) Inventors: William J. Rutter, San Francisco, CA (US); Michele P. Calos, Burlingame, CA (US); Jimmy Z. Zhang, San Francisco, CA (US)

(21) Appl. No.: 12/301,992

(22) PCT Filed: May 22, 2007
(86) PCT No.: PCT/US07/69482

§ 371 Date: Dec. 15, 2010

Related U.S. Application Data
(60) Provisional application No. 60/802,719, filed on May 22, 2006.

(51) Int. Cl. C12N 15/87; C12N 9/00; C12N 5/10; C12N 15/63

(52) U.S. Cl. 435/462; 435/183; 435/325; 435/352; 435/358; 435/366; 435/320.1

(57) ABSTRACT

The subject invention provides a site-specific integration system and methods for generating eukaryotic cells lines for protein production. The provided system includes a first site-specifically integrating target vector and a second site-specifically integrating donor vector comprising a gene of interest. Also provided are mammalian cell lines produced by the subject methods and systems, as well as kits that include the subject systems.

### CROSS REFERENCE

This application claims the benefit of U.S. Provisional Application No. 60/802,719, filed May 22, 2006, which application is incorporated herein by reference in its entirety.

### BACKGROUND OF THE INVENTION

Proteins, such as antibodies, are emerging as therapeutic and/or preventive options for a wide variety of diseases. For example, administration of therapeutic antibodies provides an important strategy for treatment and/or prophylaxis of individuals with cancer or individuals that have been exposed to, or have been infected by, viral disease agents.

However, the current process of generating cell lines that produce high levels of recombinant proteins, such as antibodies, requires labor-intensive cloning and screening steps. The identification of a cell line that is capable of producing a high yield of proteins is a tedious and time consuming process that requires the screening of hundreds of cell lines. This selection process hinders the potential to screen numerous protein therapeutic or prophylactic candidates. Moreover, the selection process also slows down the manufacture of proteins in a timely and cost-effective manner.

Most of the current mammalian cell lines expressing therapeutic proteins, such as antibodies, are developed by random genomic integration of transgenes encoding the protein. However, the random integration approach has significant drawbacks. For example, since the expression of the transgene depends on the chromosome context at the site of integration, integration of the transgene in an undesirable location results in relatively low expression of the transgene. In addition, the integration is prone to excision during passage of the “permanently” transfected cells. Furthermore, expression of the transgene often becomes “silenced” as a result of the random integration of the transgene in an undesirable location in the chromosome.

Therefore, a method for rapidly generating and identifying stable cell lines that are capable of producing high levels of recombinant proteins for use as therapeutics and diagnostics is necessary. The present invention addresses this need.

### Relevant Literature

Thyagarajan et al., Mol Cell Biol 21, 3926-34 (2001); Groth et al., Proc Natl Acad Sci USA 97, 5995-6000 (2000); Groth et al., J Mol Biol 335, 667-78 (2004); Olivares et al., Nat Biotechnol 20, 1124-8 (2002); Ortiz-Urda et al., Nat Med 8, 1166-70 (2002); Ortiz-Urda et al., Hum Gene Ther 14, 923-8 (2003); Ortiz-Urda et al. J Clin Invest 111, 251-5 (2003); Thyagarajan et al., Methods Mol Bio 308, 99-106 (2005); Olivares et al., Gene 278, 167-76 (2001); Urlaub et al., Proc Natl Acad Sci U S A 77, 4216-20 (1980); Traggiai et al., Nat Med 10, 871-5 (2004); Wurm et al., Nat Biotechnol 22, 1393-8 (2004); Andersen et al., Curr Opin Biotechnol 13, 117-23 (2002); Wirth et al., Gene 73, 419-26 (1988); Kim et al., Biotechnol Bioeng 58, 73-84 (1998); Gandor et al., FEBS Lett 377, 290-4 (1995); Kito et al., Appl Microbiol Biotechnol 60, 442-8 (2002); Coquelle et al., Cell 89, 215-25 (1997); Stark et al., Cell 57, 901-8 (1989); Wurm et al., Ann N Y Acad Sci 782, 70-8 (1996); Wurm et al., Biologicals 22, 95-102 (1994); Kim et al., Biotechnol Prog 17, 69-75 (2001); Chappell et al., J Biol Chem 278, 33793-800 (2003); Owens et al., Proc Natl Acad Sci USA 98, 1471-6 (2001); Chappell et al., Proc. Natl. Acad. Sci. U.S.A., 97, 1536-1541 (2000); Weber et al., Nat Biotechnol 22, 1440-4 (2004); Weber et al., Metab Eng 7, 174-81 (2005); Chalberg et al., J Mol Biol, 357, 28-48 (2006); Jones et al., Biotechnol Prog 19, 163-8 (2003); Marks, et al., J Mol Biol 222, 581-97 (1991); Sblattero, et al., Immunotech 3, 271-8 (1998); and Yamanaka, et al., J Biochem 117, 1218-27 (1995).

### SUMMARY OF THE INVENTION

The subject invention provides a site-specific integration system and methods for generating eukaryotic cells lines for protein production. The provided system includes a first site-specifically integrating target vector and a second site-specifically integrating donor vector comprising a gene of interest. Also provided are eukaryotic cell lines produced by the subject methods and systems, as well as kits that include the subject systems.

A feature of the present invention provides a site-specifically integrating target vector that includes a first vector recombination site that recombines with a genomic recombination site in the presence of a first unidirectional site-specific recombinase; a second vector recombination site that recombines with a donor recombination site in the presence of a second unidirectional site-specific recombinase that is different from the first unidirectional site-specific recombinase; a first portion of a first selectable marker adjacent to the 3′ end of the second vector recombination site; and a second selectable marker that is different from the first selectable marker.

In some embodiments, the genomic recombination site is a eukaryotic genomic recombination site. In some embodiments, the first vector recombination site is a bacterial genomic recombination site (attB) or a phage genomic recombination site (attP). In other embodiments, the first vector recombination site is a bacterial genomic recombination site (attB) and the genomic recombination site is a pseudo-phage genomic recombination site (pseudo-attP). In certain embodiments, the first vector recombination site is a phage genomic recombination site (attP) and the genomic recombination site is a pseudo-bacterial genomic recombination site (pseudo-attB). In other embodiments, the first vector recombination site is a pseudo-bacterial genomic recombination site (pseudo-attB) or a pseudo-phage genomic recombination attP site (pseudo-attP). In some embodiments, the second vector recombination site is a bacterial genomic recombination site (attB) or a phage genomic recombination site (attP). In some embodiments, the second vector recombination site is a pseudo-bacterial genomic recombination site (pseudo-attB) or a pseudo-phage genomic recombination attP site (pseudo-attP).

In some embodiments, the first unidirectional site-specific recombinase is a φC31 phage recombinase, a TP901-1 phage recombinase, a R4 phage recombinase, a φFC1 phage recombinase, a φRv1 phage recombinase, or a φBT1 phage recombinase. In certain embodiments, the first unidirectional site-specific recombinase is a φC31 phage recombinase. In certain embodiments, the second unidirectional site-specific recombinase is a R4 phage recombinase. In certain embodiments, a φC31 phage recombinase includes an altered φC31 phage recombinase, a TP901-1 phage recombinase includes an altered TP901-1 phage recombinase, and a R4 phage recombinase includes an altered R4 phage recombinase.

Another feature of the present invention provides a method of site-specifically integrating a polynucleotide encoding a protein of interest in a genome of a eukaryotic cell by introducing the target vector into a eukaryotic cell comprising a first unidirectional site-specific recombinase and maintaining the cell under conditions sufficient for a recombination event mediated by the first unidirectional site-specific recombinase between the first vector recombination site and the genomic recombination site to site-specifically integrate the target vector into the genome of the cell; introducing a donor vector into the target cell comprising a second unidirectional site-specific recombinase, wherein the donor vector comprises the polynucleotide encoding a protein of interest and a donor recombination site, and maintaining the target cell under conditions sufficient for a recombination event mediated by the second unidirectional site-specific recombinase between the donor recombination site and the second vector recombination site of the target vector to site-specifically integrate the polynucleotide encoding a protein of interest in the genome of the cell; wherein the first unidirectional site-specific recombinase is different from the second unidirectional site-specific recombinase. In further embodiments, the method includes selecting a cell that expresses the protein of interest.

In some embodiments, the first vector recombination site is a bacterial genomic recombination site (attB) or a phage genomic recombination site (attP). In other embodiments, the first vector recombination site is a bacterial genomic recombination site (attB) and the genomic recombination site is a pseudo-phage genomic recombination site (pseudo-attP). In certain embodiments, the first vector recombination site is a phage genomic recombination site (attP) and the genomic recombination site is a pseudo-bacterial genomic recombination site (pseudo-attB). In other embodiments, the first vector recombination site is a pseudo-bacterial genomic recombination site (pseudo-attB) or a pseudo-phage genomic recombination attP site (pseudo-attP). In some embodiments, the second vector recombination site is a bacterial genomic recombination site (attB) or a phage genomic recombination site (attP). In other embodiments, the second vector recombination site is a pseudo-bacterial genomic recombination site (pseudo-attB) or a pseudo-phage genomic recombination attP site (pseudo-attP). In some embodiments, the donor recombination site is a bacterial genomic recombination site (attB) or a phage genomic recombination site (attP). In some embodiments, the donor recombination site is a pseudo-bacterial genomic recombination site (pseudo-attB) or a pseudo-phage genomic recombination attP site (pseudo-attP).

In some embodiments, the first unidirectional site-specific recombinase is a φC31 phage recombinase, a TP901-1 phage recombinase, a R4 phage recombinase, a φFC1 phage recombinase, a φRv1 phage recombinase, or a φBT1 phage recombinase. In certain embodiments, the first unidirectional site-specific recombinase is a φC31 phage recombinase. In certain embodiments, the second unidirectional site-specific recombinase is a R4 phage recombinase. In some embodiments the protein is an enzyme that can be used for the production of nutrients or for performing enzymatic reactions in chemistry, or a polypeptide useful and valuable as a nutrient or for the treatment of a human or animal disease or for the prevention thereof, for example a hormone, a polypeptide with immunomodulatory activity, anti-viral and/or anti-tumor properties (e.g., maspin), an antibody, a viral antigen, a vaccine, a clotting factor, an enzyme inhibitor, a foodstuff ingredient, and the like. In certain embodiments, the protein is a secreted protein, such as an antibody. In some embodiments, the cell is a mammalian cell. In some embodiments, the mammalian cell is a rodent cell, such as a CHO cell or a dihydrofolate reductase-deficient CHO-derived cell line such as DG44. In other embodiments, the mammalian cell is a human cell, such as a PER.C6™ cell.

Yet another feature of the present invention provides an isolated cell, that includes a genomically integrated polynucleotide cassette comprising a first hybrid recombination site and a second hybrid recombination site flanking a vector recombination site that recombines with a donor recombination site in the presence of a unidirectional site-specific recombinase; a first portion of a first selectable marker adjacent to the vector recombination site's 3′ end; and a second selectable marker that is different from the first selectable marker.

In some embodiments, the vector recombination site is a bacterial genomic recombination site (attB) or a phage genomic recombination site (attP). In some embodiments, the donor recombination site is a bacterial genomic recombination site (attB) or a phage genomic recombination site (attP). In some embodiments, the unidirectional site-specific recombinase is a φC31 phage recombinase, a TP901-1 phage recombinase, or a R4 phage recombinase. In some embodiments, the cell is a mammalian cell. In some embodiments, the mammalian cell is a rodent cell, such as a CHO cell or a dihydrofolate reductase-deficient CHO-derived cell line such as DG44. In other embodiments, the mammalian cell is a human cell, such as a PER.C6™ cell.

Yet another feature of the present invention provides a kit for use in site-specifically integrating a polynucleotide into a genome of a cell in vitro, including: a target vector; and a donor vector that includes two promoters, two signal sequences if the protein of interest is secreted, 2 gene regulatory switches to control gene expression, two translational enhancers to increase expression, two multiple cloning sites, a donor recombination site, and a second portion of a first selectable marker (e.g., promoter) adjacent to the donor recombination site's 5′ end. In some embodiments, the kit further includes a first unidirectional site-specific recombinase or nucleic acid encoding the same. In further embodiments, the kit also includes a second unidirectional site-specific recombinase or nucleic acid encoding the same that is different from the first unidirectional site-specific recombinase.

In some embodiments the first unidirectional site-specific recombinase is a φC31 phage recombinase, a TP901-1 phage recombinase, a R4 phage recombinase, a φFC1 phage recombinase, a φRv1 phage recombinase, or a φBT1 phage recombinase. In some embodiments, the second unidirectional site-specific recombinase is a φC31 phage recombinase, a TP901-1 phage recombinase, a R4 phage recombinase, a φFC1 phage recombinase, a φRv1 phage recombinase, or a φBT1 phage recombinase.

Yet another feature of the present invention provides a kit for use in producing a protein in a eukaryotic cell, including: an isolated eukaryotic cell, that includes a genomically integrated polynucleotide cassette comprising a first hybrid recombination site and a second hybrid recombination site flanking a vector recombination site that recombines with a donor recombination site in the presence of a unidirectional site-specific recombinase, a first portion of a first selectable marker adjacent to the vector recombination site's 3′ end, and a second selectable marker that is different from the first selectable marker; and a donor vector that includes a multiple cloning site, a donor recombination site, and a second portion of a first selectable marker (e.g., promoter) adjacent to the donor recombination site's 5′ end.

In some embodiments, the kit also includes a unidirectional site-specific recombinase or nucleic acid encoding the same. In some embodiments the unidirectional site-specific recombinase is a φC31 phage recombinase, a TP901-1 phage recombinase, a R4 phage recombinase, a φFC1 phage recombinase, a φRv1 phage recombinase, or a φBT1 phage recombinase.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a schematic representation of an exemplary target vector. The exemplary target vector includes a first vector recombination site (e.g., a φC31 attB site), a second vector recombination site (e.g., R4 attP site), a first portion of a first selectable marker (e.g., promoter-less first selectable marker (e.g., zeocin resistance gene)) downstream of the R4 attP site, and a second selectable marker (e.g., a hygromycin resistance gene).

FIG. 2 is a schematic representation of an exemplary donor vector. The exemplary donor vector includes a donor recombination site (e.g., R4 attB site) a gene of interest and a promoter (e.g., a CMV promoter) just upstream of the R4 attB site.

FIG. 3 is a schematic representation of an exemplary initial site-specific integration event between the φC31 attB site present on the target vector and the φC31 pseudo-attP site present in the genome of the target cell. The integration event is mediated by the φC31 integrase.

FIG. 4 is a schematic representation of an exemplary site-specific integration event between the R4 attB site present on the donor vector and the R4 attP integrated into the cell genome as a result of integration of the target vector. The second integration event is mediated by the R4 integrase

FIG. 5 is a schematic representation of an exemplary DHFR-target vector. The exemplary DHFR-target vector includes an R4 attP site, a φC31 attB site, a hygromycin resistance gene, a DHFR gene, and a first portion (e.g., promoter-less) of a zeocin resistance gene downstream of the R4 attP site.

FIG. 6 is a schematic representation of an exemplary DHFR-donor vector. The exemplary donor vector includes an R4 attB site, a gene of interest, a DHFR gene, and a CMV promoter just upstream of the R4 attB site.

FIG. 7 is a schematic representation of an exemplary IRES-donor vector. The exemplary donor vector includes an R4 attB site, a gene of interest, a CMV promoter just upstream of the R4 attB site, and an IRES between the transcription start site and the coding region for the gene of interest.

FIG. 8 is a schematic representation of the target vector pR1. The target vector pR1 includes a first vector recombination site (e.g., a R4 attB 295 site), a second vector recombination site (e.g., a φC31 attP 103 site), a first portion of a first selectable marker (e.g., promoter-less selectable marker (e.g., puromycin resistance gene)) downstream of the φC31 attP 103 site, and a complete second selectable marker (e.g., a hygromycin resistance gene cassette). It also contains a ColE1 origin of DNA replication and an ampicillin resistance gene cassette for maintenance and selection in E. coli, respectively. Asterisks designate unique restriction enzyme sites.

FIG. 9 is a schematic representation of an exemplary donor expression vector backbone (pHPC-4). The exemplary donor expression vector backbone includes a donor recombination site (e.g., a φC31 attB 285 AAA site), two CMV promoters, two signal sequences for secretion of proteins, two polylinkers for insertion of genes of interest, and two bovine growth hormone poly adenylation signals. It also includes a weaker promoter (e.g., a SV40 promoter) just upstream of the φC31 attB 285 AAA site for selecting integration of a donor expression vector into the target vector. In addition, the vector also includes a ColE1 origin of DNA replication and an ampicillin resistance gene cassette for maintenance and selection in E. coli, respectively. Asterisks designate unique restriction enzyme sites.

FIG. 10 is a schematic representation of an exemplary donor expression vector (pD1-DTX-1). The exemplary donor expression vector includes a donor recombination site (e.g., a φC31 attB 285 AAA site), two CMV promoters, two signal sequences, the heavy and light chains of an anti-diphtheria toxin antibody, and two bovine growth hormone polyadenylation signals. The vector also includes a weaker promoter (e.g., a SV40 promoter) just upstream of the φC31 attB 285 AAA site for selecting integration of the donor expression vector into the target vector. In addition, the vector also includes a ColE1 origin of DNA replication and an ampicillin resistance gene cassette for maintenance and selection in E. coli, respectively.

FIG. 11 is a schematic representation of the rapid testing procedure used to verify the function of each of the four vectors used to generate cell lines for high level protein production. The first step uses the R4 integrase encoded by an R4 integrase expression vector (e.g., pCMV sre to mediate integration of the target vector into R4 pseudo attP sites. Forty eight hours are allowed for integration to occur without selection (e.g., hygromycin selection).

The second step uses a φC31 mutant integrase encoded by a φC31 mutant integrase expression vector (e.g., pCS-M3J) to mediate integration of the donor vector into the target vector. Forty eight hours are allowed for integration to occur and then a puromycin selection is used to isolate a stable pool of cells. These cells are analyzed for protein expression. High level protein expression depends on proper function of each of the four plasmids used. Whether or not the target vector integrated randomly or site-specifically at R4 pseudo attP sites in the first step can be assessed by doing the experiment with or without the R4 integrase expression vector. The level of protein expression will be substantially lower if the R4 integrase expression vector is omitted because unintegrated target vectors will be diluted out as the cells divide over the length of the experiment (>17 days).

FIG. 12 is a schematic representation of an exemplary first site-specific integration event between the R4 attB 295 site present on the target vector and the R4 pseudo-attP sites present in the genome of the target cell. The integration event is mediated by the R4 integrase, encoded by the plasmid pCMV sre. Hygromycin selection is used to isolate stable clones (e.g., PER.C6-φC31 attP or DG44-φC31 attP cell lines) with the target vector integrated at R4 pseudo-attP sites.

FIG. 13 is a schematic representation of an exemplary second site-specific integration event that occurs in φC31 attP cell lines between the φC31 attB 285 AAA site present on the donor vector and the φC31 attP 103 site integrated into the cell genome as a result of integration of the target vector. The second integration event is mediated by a φC31 mutant integrase (e.g., a mutant φC31 integrase encoded by the plasmid pCS-M3J). A reconstituted drug resistance expression cassette is used to select for integrants in which the donor expression vector has integrated into the target vector, and to select against those cell lines in which the donor vector has integrated into φC31 pseudo-attP sites.

FIG. 14 diagrams the sequences of the φC31 attB, attP, and attL 88 sites. The sequences of the wild type φC31 attB and φC31 attP are given in the top half. The underlined sequence in the top half indicates the sequences from attB and attP which would form an attL site after recombination. By convention attL is named according to the side of the recombination cross over point that was derived from attB. For example in attL, sequences on the left side of the recombination cross over point are derived from sequences on the left (5′) side of the recombination cross over point of attB. Sequences in attL on the right side of the recombination cross over point are derived from sequences on the right (3′) side of the recombination cross over point of attP.

The bottom half of the figure diagrams how the attB and attP sequences were modified to make the φC31 attP 103 and φC31 attB 285 AAA sites that were used on the target and donor vectors, respectively. It also indicates the sequence of the φC31 attL 88 site that results after the φC31 attB 285 AAA site in the donor vector integrates into the φC31 attP 103 site in the target vector.

FIG. 15 is a schematic representation of an exemplary target-DHFR vector (pR1-DHFR). The exemplary target-DHFR vector includes a φC31 attP 103 site, an R4 attB 295 site, a hygromycin resistance gene, a DHFR gene, and a first portion of a (e.g., promoter-less) puromycin resistance gene downstream of the φC31 attP103 site. The vector also includes a ColE1 origin of DNA replication and an ampicillin resistance gene cassette for maintenance and selection in E. coli, respectively.

FIG. 16 is a schematic representation of an exemplary donor-DHFR expression vector (pD1-DHFR). The exemplary donor-DHFR expression vector includes a donor recombination site (e.g., a φC31 attB 285 AAA site), two CMV promoters, two signal sequences, the heavy and light chains of an anti-diphtheria toxin antibody, two bovine growth hormone polyadenylation signals, the DHFR expression cassette, and a promoter (e.g., a SV40 promoter) just upstream of the φC31 attB 285 AAA site for selecting integration of the donor vector into the target vector. The vector also includes a ColE1 origin of DNA replication and an ampicillin resistance gene cassette for maintenance and selection in E. coli, respectively.

FIG. 17 is a schematic representation of an exemplary IRES-donor expression vector (pD1-IRES). The exemplary IRES-donor expression vector includes a donor recombination site (e.g., a φC31 attB 285 AAA site), two CMV promoters, two internal ribosome entry sites (IRES) in the 5′ untranslated region, two signal sequences, the heavy and light chains of an anti-diphtheria toxin antibody, two bovine growth hormone polyadenylation signals, and a promoter (e.g., a SV40 promoter) just upstream of the φC31 attB 285 AAA site for selecting integration of the donor vector into the target vector. The vector also includes a ColE1 origin of DNA replication and an ampicillin resistance gene cassette for maintenance and selection in E. coli, respectively.

FIG. 18 is a schematic representation of an exemplary regulating target vector (pR1reg). The exemplary regulating target vector includes a first vector recombination site (e.g., a R4 attB 295 site), a second vector recombination site (e.g., a φC31 attP 103 site), a first portion of a first selectable marker (e.g., promoter-less selectable marker (e.g., puromycin resistance gene)) downstream of the φC31 attP 103 site, a complete second selectable marker (e.g., a hygromycin resistance gene cassette), and a cassette that encodes proteins (e.g., RheoActivator and RheoReceptor) capable of conferring controllable gene regulation on one or more genes present on a regulatable donor expression vector (e.g., pD1reg), which has genes that are configured in a manner such that they are capable of being regulated. The vector also includes a ColE1 origin of DNA replication and an ampicillin resistance gene cassette for maintenance and selection in E. coli, respectively.

FIG. 19 is a schematic representation of an exemplary regulating target-DHFR vector (pR1reg-DHFR). The exemplary regulating target-DHFR vector includes a first vector recombination site (e.g., a R4 attB 295 site), a second vector recombination site (e.g., a φC31 attP 103 site), a first portion of a first selectable marker (e.g., promoter-less selectable marker (e.g., puromycin resistance gene)) downstream of the φC31 attP 103 site, a complete second selectable marker (e.g., a hygromycin resistance gene cassette), a DHFR gene, and a cassette that encodes proteins (e.g., RheoActivator and RheoReceptor) capable of conferring controllable gene regulation on one or more genes present on a regulatable donor expression vector (e.g., pD1reg), which has genes that are configured in a manner such that they are capable of being regulated. The vector also includes a ColE1 origin of DNA replication and an ampicillin resistance gene cassette for maintenance and selection in E. coli, respectively.

FIG. 20 is a schematic representation of an exemplary regulatable donor expression vector backbone (pD1reg). The exemplary regulatable donor expression vector backbone includes a donor vector recombination site (e.g., a φC31 attB 285 AAA site), two sequences to prevent read-through transcription into the gene regulatory sequences (e.g., a SV40 polyadenylation region), two sequences that mediate gene regulation (e.g., 5×GAL4 UAS, TATA box, and a 5′ UTR), two signal sequences, a polylinker for inserting genes of interest, two bovine growth hormone polyadenylation signals, and a promoter (e.g., a SV40 promoter) just upstream of the φC31 attB 285 AAA site for selecting integration of the donor vector into the target vector. The vector also includes a ColE1 origin of DNA replication and an ampicillin resistance gene cassette for maintenance and selection in E. coli, respectively. Asterisks designate unique restriction enzyme sites.

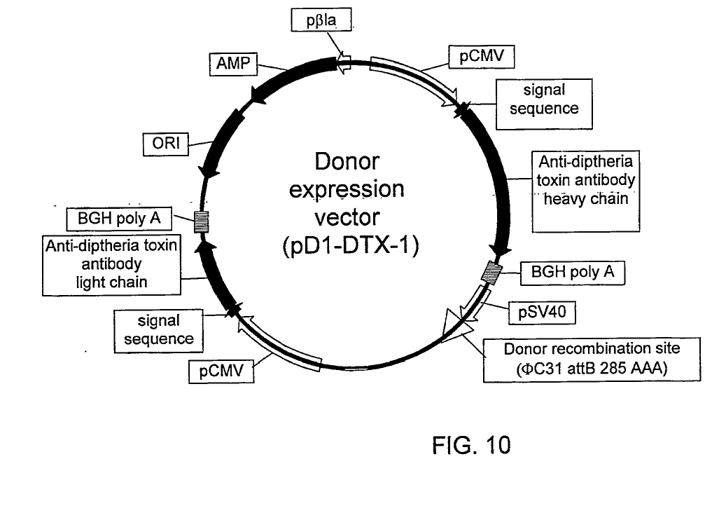

FIG. 21 is a schematic representation of an exemplary selectable donor expression vector (pD1-DTX1-G418). The exemplary selectable donor expression vector includes all of the elements of a donor expression vector (FIG. 10), but also includes a complete selectable marker gene (e.g, G418).

FIG. 22 demonstrates site-specific recombination of a target vector with a donor expression vector after transient transfection.

FIG. 23 shows the sequence of an R4 pseudo att site isolated from cells in which a target vector was site-specifically integrated using R4 integrase. The R4 core sequence in which recombination occurs is shown in upper case letters.

FIG. 24 shows sequences of hybrid φC31 att sites isolated from DG44 cells in which a donor expression vector was site-specifically integrated into a target vector. Panel A shows the hybrid attL site and Panel B shows the hybrid attR site. The top nucleic acid sequence shows the predicted sequence of the donor expression vector region, followed by the attL, and then the puromycin resistance sequence, which originated from the target vector. The bottom sequence is the actual sequence from the cell line. As shown in the figure the actual nucleic acid sequence corresponds exactly with the predicted sequence.

FIG. 25 shows sequences of hybrid φpC31 att sites isolated from PER.C6™ cells in which a donor expression vector was site-specifically integrated into a target vector. Panel A shows the hybrid attL site and Panel B shows the hybrid attR site. The top nucleic acid sequence shows the predicted sequence of the donor expression vector region, followed by the attL, and then the puromycin resistance sequence, which originated from the target vector. The bottom seqeuence is the actual sequence from the cell line. As shown in the figure the actual nucleic acid sequence corresponds exactly with the predicted sequence.

FIG. 26 shows polymerase chain reaction-mediated amplification of attB (Panel A) and attR (Panel B) sites from the genomic DNA of cells with site-specifically integrated donor expression vectors.

FIG. 27A shows expression of an antibody from CHO dhfr-pool of clones after site-specific donor expression vector integration.

FIG. 27B shows expression of an antibody from PER.C6™ pool of clones after site-specific donor expression vector integration.

FIGS. 28A and 28B show expression of an antibody from single cell clones of CHO dhfr-pool #2G7 that contain site-specifically integrated donor expression vectors.

FIG. 29 shows expression of an antibody (pg/cell/day) from a pool of cells in which a donor expression vector was site-specifically integrated into a DHFR-target vector and cell populations were then exposed to increasing concentrations of methotrexate.

FIG. 30 is a schematic representation of an exemplary reporter donor expression vector (pD3-DTX1). The exemplary reporter donor expression vector includes all of the elements of a donor expression vector (FIG. 10), but also includes a gene encoding a reporter molecule, such as green fluorescent protein. The presence of the reporter gene enables easy identification of individual cells that express a protein of interest.

FIG. 31 shows comparable specific binding activity of anti-diphtheria toxin antibody expressed in DG44 cells and PER.C6™ cells.

FIG. 32 shows the biological, in vitro neutralizing activity of anti-diphtheria toxin antibody expressed from DG44 cells or PER.C6™ cells compared to that from the human B-cell line (D2.2), from which the antibody genes were cloned.

FIGS. 33A-33B show the nucleic acid sequence for the pR1 vector.

FIGS. 34A-34C show the nucleic acid sequence for the pD1-DTX-1 vector.

FIGS. 35A-35C show the nucleic acid sequence for the pR1-DHFR vector.

FIGS. 36A-36D show the nucleic acid sequence for the pD1-DTX1-G418 vector.

FIGS. 37A-37D show the nucleic acid sequence for the pD3-DTX1 vector.

### DEFINITIONS

“Recombinases” are a family of enzymes that mediate site-specific recombination between specific DNA sequences recognized by the recombinase (Esposito, D., and Scocca, J. J., Nucleic Acids Research 25, 3605-3614 (1997); Nunes-Duby, S. E., et al., Nucleic Acids Research 26, 391-406 (1998); Stark, W. M., et al., Trends in Genetics 8, 432-439 (1992)). Within this group are several subfamilies including “Integrase” or tyrosine recombinase (including, for example, Cre and lambda integrase) and “Resolvase/Invertase” or serine recombinase (including, for example, φC31 integrase, R4 integrase, and TP-901 integrase). The term also includes recombinases that are altered as compared to wild-type, for example as described in U.S. Patent Publication 20020094516, the disclosure of which is hereby incorporated by reference in its entirety herein.

A “unidirectional site-specific recombinase” is a naturally-occurring recombinase, such as the φC31 integrase, a mutated or altered recombinase, such as a mutated or altered φC31 integrase that retains unidirectional, site-specific recombination activity, or a bi-directional recombinase modified so as to be unidirectional, such as a cre recombinase that has been modified to become unidirectional.

“Altered recombinases” and “mutant recombinases” are used interchangeably herein to refer to recombinase enzymes in which the native, wild-type recombinase gene found in the organism of origin has been mutated in one or more positions relative to a parent recombinase (e.g., in one or more nucleotides, which may result in alterations of one or more amino acids in the altered recombinase relative to a parent recombinase). “Parent recombinase” is used to refer to the nucleotide and/or amino acid sequence of the recombinase from which the altered recombinase is generated. The parent recombinase can be a naturally occurring enzyme (i.e., a native or wild-type enzyme) or a non-naturally occurring enzyme (e.g., a genetically engineered enzyme). Altered recombinases of interest in the invention exhibit a DNA binding specificity and/or level of activity that differs from that of the wild-type enzyme or other parent enzyme. Such altered binding specificity permits the recombinase to react with a given DNA sequence differently than would the parent enzyme, while an altered level of activity permits the recombinase to carry out the reaction at greater or lesser efficiency. A recombinase reaction typically includes binding to the recognition sequence and performing concerted cutting and ligation, resulting in strand exchanges between two recombining recognition sites.

“Site-specific integration” or “site-specifically integrating” as used herein refers to the sequence specific recombination and integration of a first nucleic acid with a second nucleic acid, typically mediated by a recombinase. In general, site-specific recombination or integration occurs at particular defined sequences recognized by the recombinase. In contrast to random integration, site specific integration occurs at a particular sequence (e.g., a recombinase attachment site) at a higher efficiency.

The native attB and attP recognition sites of phage φC31 (i.e. bacteriophage φC31) are generally about 34 to 40 nucleotides in length (Groth et al. Proc Natl Acad Sci USA 97:5995-6000 (2000)). These sites are typically arranged as follows: AttB comprises a first DNA sequence attB5′, a core region, and a second DNA sequence attB3′, in the relative order from 5′ to 3′ attB5′-core region-attB3′. AttP comprises a first DNA sequence attP5′, a core region, and a second DNA sequence attP3′, in the relative order from 5′ to 3′ attP5′-core region-attP3′. The core region of attP and attB of φC31 has the sequence 5′-TTG-3′. Other phage integrases (such as the R4 phage integrase) and their recognition sequences can be adapted for use in the invention.

Action of the integrase upon these recognitions sites is unidirectional in that the enzymatic reaction produces nucleic acid recombination products that are not effective substrates of the integrase. This results in stable integration with little or no detectable recombinase-mediated excision, i.e., recombination that is “unidirectional”. The recombination product of integrase action upon the recognition site pair comprises, for example, in order from 5′ to 3′: attB5′-recombination product site sequence-attP3′, and attP5′-recombination product site sequence-attB3′. Thus, where the target vector comprises an attB site and the target genome comprises an attP sequence, a typical recombination product comprises the sequence (from 5′ to 3′): attP5′-TTG-attB3′ {targeting vector sequence}attB5′-TTG-attP3′. Because the attB and attP sites are different sequences, recombination results in a hybrid site-specific recombination site (designated attL or attR for left and right) that is neither an attB sequence or an attP sequence, and is functionally unrecognizable as a site-specific recombination site (e.g., attB or attP) to the relevant unidirectional site-specific recombinase, thus removing the possibility that the unidirectional site-specific recombinase will catalyze a second recombination reaction between the attL and the attR that would reverse the first recombination reaction.

A “native recognition site”, as used herein, means a recognition site that occurs naturally in the genome of a cell (i.e., the sites are not introduced into the genome, for example, by recombinant means).

A “wild-type recombination site” as used herein means a recombination site normally used by an integrase or recombinase. For example, lambda is a temperate bacteriophage that infects E. coli. The phage has one attachment site for recombination (attP) and the E. coli bacterial genome has an attachment site for recombination (attB). Both of these sites are wild-type recombination sites for lambda integrase. In the context of the present invention, wild-type recombination sites occur in the homologous phage/bacteria system. Accordingly, wild-type recombination sites can be derived from the homologous system and associated with heterologous sequences, for example, the attB site can be placed in other systems to act as a substrate for the integrase.

A “pseudo-site” or a “pseudo-recombination site” as used herein means a DNA sequence comprising a recognition site that is bound by a recombinase enzyme where the recognition site differs in one or more nucleotides from a wild-type recombinase recognition sequence and/or is present as an endogenous sequence in a genome that differs from the sequence of a genome where the wild-type recognition sequence for the recombinase resides. For a given recombinase, a pseudo-recombination sequence is functionally equivalent to a wild-type recombination sequence, occurs in an organism other than that in which the recombinase is found in nature, and may have sequence variation relative to the wild type recombination sequences. In some embodiments a “pseudo attP site” or “pseudo attB site” refer to pseudo sites that are similar to the recognitions site for wild-type phage (attP) or bacterial (attB) attachment site sequences, respectively, for phage integrase enzymes, such as the phage φC31. In many embodiments of the invention the pseudo attP site is present in the genome of a host cell, while the wild type ttB site is present on a targeting vector in the system of the invention. “Pseudo att site” is a more general term that can refer to either a pseudo attP site or a pseudo attB site. It is understood that att sites or pseudo att sites may be present on linear or circular nucleic acid molecules. In certain embodiments, the presence of “pseudo-recombination sites” in the genome of the target cell avoids the need for introducing a recombination site into the genome.

A “hybrid-recombination site”, as used herein, refers to a recombination site constructed from portions of wild type and/or pseudo-recombination sites. As an example, a wild-type recombination site may have a short, core region flanked by palindromes. In one embodiment of a “hybrid-recombination site” the sequence 5′ of the core region sequence of the hybrid-recombination site matches a pseudo-recombination site and the sequence 3′ of the core of the hybrid-recombination site match the wild-type recombination site. In an alternative embodiment, the hybrid-recombination site may be comprised of the region 5′ of the core from a wild-type attB site and the region 3′ of the core from a wild-type attP recombination site, or vice versa. Other combinations of such hybrid-recombination sites will be evident to those having ordinary skill in the art, in view of the teachings of the present specification.

By “nucleic acid fragment of interest” it is meant any nucleic acid fragment adapted for insertion into a genome. Suitable examples of nucleic acid fragments of interest include promoter elements, therapeutic genes, marker genes, control regions, trait-producing fragments, nucleic acid elements to accomplish gene disruption, and the like.

Methods of transfecting cells are well known in the art. By “transfected” it is meant an alteration in a cell resulting from the uptake of foreign nucleic acid, usually DNA. Use of the term “transfection” is not intended to limit introduction of the foreign nucleic acid to any particular method. Suitable methods include viral infection, conjugation, electroporation, particle gun technology, calcium phosphate precipitation, direct microinjection, and the like. The choice of method is generally dependent on the type of cell being transfected and the circumstances under which the transfection is taking place (i.e. in vitro, ex vivo, or in vivo). A general discussion of these methods can be found in Ausubel, et al, Short Protocols in Molecular Biology, 3rd ed., Wiley & Sons, 1995.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term polynucleotide sequence is the alphabetical representation of a polynucleotide molecule. This alphabetical representation can be input into databases in a computer having a central processing unit and used for bioinformatics applications such as functional genomics and homology searching.

A “coding sequence” or a sequence that “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and even synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences that are immunologically identifiable with a polypeptide encoded by the sequence.

“Operably linked” refers to an arrangement of elements wherein the components so described are configured so as to perform their usual function. Thus, a given promoter that is operably linked to a coding sequence (e.g., a reporter expression cassette) is capable of effecting the expression of the coding sequence when the proper enzymes are present. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

By “genomic domain” is meant a genomic region that includes one or more, typically a plurality of, exons, where the exons are typically spliced together during transcription to produce an mRNA, where the mRNA often encodes a protein product, e.g., a therapeutic protein, etc. In many embodiments, the genomic domain includes the exons of a given gene, and may also be referred to herein as a “gene.” Modulation of transcription of the genomic domain pursuant to the subject methods results in at least about 2-fold, sometimes at least about 5-fold and sometimes at least about 10-fold modulation, e.g., increase or decrease, of the transcription of the targeted genomic domain as compared to a control, for those instances where at least some transcription of the targeted genomic domain occurs in the control. For example, in situations where a given genomic domain is expressed at only low levels in a non-modified target cell (used as a control), the subject methods may be employed to obtain an at least 2-fold increase in transcription as compared to a control. Transcription levels can be determined using any convenient protocol, where representative protocols for determining transcription levels include, but are not limited to: RNA blot hybridization, RT PCR, RNAse protection and the like.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as integrating vectors.

An “expression cassette” comprises any nucleic acid construct capable of directing the expression of a gene/coding sequence of interest. Such cassettes can be constructed into a “vector,” “vector construct,” “expression vector,” or “gene transfer vector,” in order to transfer the expression cassette into target cells. Thus, the term includes cloning and expression vehicles, as well as viral vectors.

In the present invention, when a recombinase is “derived from a phage” the recombinase need not be explicitly produced by the phage itself, the phage is simply considered to be the original source of the recombinase and coding sequences thereof. Recombinases can, for example, be produced recombinantly or synthetically, by methods known in the art, or alternatively, recombinases may be purified from phage infected bacterial cultures.

“Substantially purified” generally refers to isolation of a substance (compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

The term “exogenous” is defined herein as DNA which is introduced into a cell by the method of the present invention, such as with the DNA constructs defined herein. Exogenous DNA can possess sequences identical to or different from the endogenous DNA present in the cell prior to transfection.

By “transgene” or “transgenic element” is meant an artificially introduced, chromosomally integrated nucleic acid sequence present in the genome of a host organism.

The term “transgenic animal” means a non-human animal having a transgenic element integrated in the genome of one or more cells of the animal. “Transgenic animals” as used herein thus encompasses animals having all or nearly all cells containing a genetic modification (e.g., fully transgenic animals, particularly transgenic animals having a heritable transgene) as well as chimeric, transgenic animals, in which a subset of cells of the animal are modified to contain the genomically integrated transgene.

“Target cell” as used herein refers to a cell that in which a genetic modification is desired. Target cells can be isolated (e.g., in culture) or in a multicellular organism (e.g., in a blastocyst, in a fetus, in a postnatal animal, and the like). Target cells of particular interest in the present application include, but not limited to, cultured mammalian cells, including CHO cells, and stem cells (e.g., embryonic stem cells (e.g., cells having an embryonic stem cell phenotype), adult stem cells, pluripotent stem cells, hematopoietic stem cells, mesenchymal stem cells, and the like).

### DETAILED DESCRIPTION OF THE INVENTION

The subject invention provides a site-specific integration system and methods for generating eukaryotic cells lines for protein production. The provided system includes a first site-specifically integrating target vector and a second site-specifically integrating donor vector comprising a gene of interest. Also provided are eukaryotic cell lines produced by the subject methods and systems, as well as kits that include the subject systems.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the vector” includes reference to one or more vectors and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Overview

In general, the present invention provides a first site-specifically integrating target vector and a second site-specifically integrating donor vector comprising a gene of interest for use in generating mammalian cells lines capable of protein production. The elements of the target vector are selected so that a first unidirectional site-specific integrase recognizes a first vector site-specific recombination site present on the target vector and a genomic site-specific recombination site in the genome of the target cell, resulting in integration of the target vector having a target site-specific recombination site for a second unidirectional site-specific integrase into the genome of the target cell.

The resulting cell line having a target site-specific recombination site for the second unidirectional site-specific integrase can then be used for efficiently generating a cell line capable of producing a desired protein. A donor vector having a polynucleotide encoding a protein of interest and a donor site-specific recombination site for the second unidirectional site-specific integrase can be introduced into the cell line, resulting in integration of the donor vector into the genome of the target cell. Since integration of the transgene can be directed in a site-specific manner, the present invention is useful for providing integration of a transgene at a desirable location and avoiding low expression of the transgene due to integration in an undesirable location.

The invention will now be described in greater detail.

### Vectors

As noted above, the system includes a target vector for integrating a site-specific recombination site into the genome of a target cell and a donor vector for integrating a polynucleotide encoding a protein of interest into the introduced site-specific recombination site. The vectors are typically circular and may also contain selectable markers, an origin of replication, and other elements such as a promoter, promoter-enhancer sequences, a selection marker sequence, an origin of replication, an inducible element sequence, an epitope tag sequence, and the like. See, e.g., U.S. Pat. No. 6,632,672, the disclosure of which is incorporated by reference herein in its entirety.

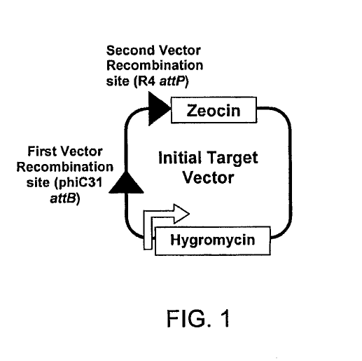

The present invention provides a target vector comprising (a) a first vector site-specific recombination site capable of recombining with a genomic recombination site in the genome of a eukaryotic cell in the presence of a first unidirectional site-specific recombinase; (b) a second vector site-specific recombination site capable of recombining with a donor site-specific recombination site on a donor vector in the presence of a second unidirectional site-specific recombinase; (c) a first portion of a first selectable marker (e.g., a promoter-less first selectable marker) adjacent to a 3′ side of the second vector site-specific recombination site; and (d) a second selectable marker that is different from the first selectable marker, and the first unidirectional site-specific recombinase is different from the second unidirectional site-specific recombinase. An exemplary target vector is provided in FIG. 1.

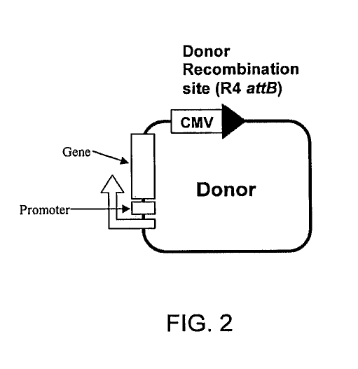

The present invention also provides a donor vector comprising (a) a multiple cloning site; (b) a donor site-specific recombination site that is capable of recombining with the second vector site-specific recombination site of the target vector in the presence of a second unidirectional site-specific recombinase; and (c) a second portion of a first selectable marker (e.g., promoter) adjacent to the 5′ side of the donor site-specific recombination site. In certain embodiments, the donor vector further comprises a polynucleotide encoding a protein of interest present in the multiple cloning site. An exemplary donor vector is provided in FIG. 2.

Two major families of unidirectional site-specific recombinases from bacteria and unicellular yeasts have been described: the integrase or tyrosine recombinase family includes Cre, Flp, R, and lambda integrase (Argos, et al., EMBO J. 5:433-440, (1986)) and the resolvase/invertase or serine recombinase family that includes some phage integrases, such as, those of phages φC31, R4, and TP901-1 (Hallet and Sherratt, FEMS Microbiol. Rev. 21:157-178 (1997)). For further description of suitable site-specific recombinases, see U.S. Pat. No. 6,632,672 and U.S. Patent Publication No. 20030050258, the disclosures of which are herein incorporated herein by reference in their entireties.

In certain embodiments, the unidirectional site-specific recombinase is a serine integrase. Serine integrases that may be useful for in vitro and in vivo recombination include, but are not limited to, integrases from phages φC31, R4, TP901-1, phiBT1, Bxb1, RV-1, A118, U153, and phiFC1, as well as others in the large serine integrase family (Gregory, Till and Smith, J. Bacteriol., 185:5320-5323 (2003); Groth and Calos, J. Mol. Biol. 335:667-678 (2004); Groth et al. PNAS 97:5995-6000 (2000); Olivares, Hollis and Calos, Gene 278:167-176 (2001); Smith and Thorpe, Molec. Microbiol., 4:122-129 (2002); Stoll, Ginsberg and Calos, J. Bacteriol., 184:3657-3663 (2002)). In addition to these wild-type integrases, altered integrases that bear mutations have been produced (Sclimenti, Thyagarajan and Calos, NAR, 29:5044-5051 (2001)). These integrases may have altered activity or specificity compared to the wild-type and are also useful for the in vitro recombination reaction and the integration reaction into the eukaryotic genome.

In representative embodiments, the first unidirectional site-specific recombinase and the second unidirectional site-specific recombinase are different. Each unidirectional site-specific recombinase has distinct site-specific recombination sites (att or attachment sites) that do not recombine with the attachment sites of other unidirectional site-specific recombinases. By using two different unidirectional site-specific recombinase in sequence, one for integration of the target vector and then the other for integration of the donor vector, there is no chance for an unwanted intramolecular recombination within the initial target vector between the attachment site for genomic integration of the target vector and the attachment site for use in integration of the donor vector. It is desirable to avoid such intramolecular recombination events because not only would they create hybrid sites that may not be able to integrate into the genome of the target cell, but they also may result in deletion of important sequence elements in the target vector.

Accordingly, the first and second unidirectional site specific recombinases should be derived from different phages, e.g., φC31, R4, TP901-1, phiBT1, Bxb1, RV-1, A118, U153, and phiFC1, or may be derived from the same phage but at least one of first and second unidirectional site-specific recombinase is an altered unidirectional site-specific recombinase as that recognizes a different site-specific recombination site than the site-specific recombination site recognized by the corresponding wild type unidirectional site-specific recombinase.

In general, site specific recombination sites recognized by a site-specific recombinase in a bacterial genome are designated bacterial attachment sites (“attB”) and the corresponding site specific recombination sites present in the bacteriophage are designated phage attachment sites (“attP”). These sites have a minimal length of approximately 34-40 base pairs (bp) Groth, A. C., et al., Proc. Natl. Acad. Sci. USA 97, 5995-6000 (2000)). These sites are typically arranged as follows: AttB comprises a first DNA sequence attB5′, a core region, and a second DNA sequence attB3′ in the relative order attB5′-core region-attB3; attP comprises a first DNA sequence (attP5′), a core region, and a second DNA sequence (attP3′) in the relative order attP5′-core region-attP3′.

For example, for the phage φC31 attP (the phage attachment site), the core region is 5′-TTG-3′ the flanking sequences on either side are represented here as attP5′ and attP3′, the structure of the attP recombination site is, accordingly, attP5′-TTG-attP3′. Correspondingly, for the native bacterial genomic target site (attB) the core region is 5′-TTG-3′, and the flanking sequences on either side are represented here as attB5′ and attB3′, the structure of the attB recombination site is, accordingly, attB5′-TTG-attB3′.

Because the attB and attP sites are different sequences, recombination results in a hybrid site-specific recombination site (designated attL or attR for left and right) that is neither an attB sequence or an attP sequence, and is functionally unrecognizable as a site-specific recombination site (e.g., attB or attP) to the relevant unidirectional site-specific recombinase, thus removing the possibility that the unidirectional site-specific recombinase will catalyze a second recombination reaction between the attL and the attR that would reverse the first recombination reaction. For example, after a single-site, φC31 integrase mediated, recombination event takes place the result is the following recombination product: attB5′-TTG-attP3′{φC31 vector sequences}attP5′-TTG-attB3′. Typically, after recombination the post-recombination recombination sites are no longer able to act as substrate for the φC31 recombinase. This results in stable integration with little or no recombinase mediated excision.

Native recombination sites have been found to exist in the genomes of a variety of organisms, where the native recombination site does not necessarily have a nucleotide sequence identical to the wild-type recombination sequences (for a given recombinase); but such native recombination sites are nonetheless sufficient to promote recombination meditated by the recombinase. Such recombination site sequences are referred to herein as “pseudo-recombination sequences.” For a given recombinase, a pseudo-recombination sequence is functionally equivalent to a wild-type recombination sequence, occurs in an organism other than that in which the recombinase is found in nature, and may have sequence variation relative to the wild type recombination sequences.

Identification of pseudo-recombination sequences can be accomplished, for example, by using sequence alignment and analysis, where the query sequence is the recombination site of interest (for example, attP and/or attB).

The genome of a target cell may be searched for sequences having sequence identity to the selected recombination site for a given recombinase, for example, the attP and/or attB of φC31 or R4. Nucleic acid sequence databases, for example, may be searched by computer. The find patterns algorithm of the Wisconsin Software Package Version 9.0 developed by the Genetics Computer Group (GCG; Madison, Wis.), is an example of a programmed used to screen all sequences in the GenBank database (Benson et al., 1998, Nucleic Acids Res. 26, 1-7). In this aspect, when selecting pseudo-recombination sites in a target cell, the genomic sequences of the target cell can be searched for suitable pseudo-recombination sites using either the attP or attB sequences associated with a particular recombinase or altered recombinase. Functional sizes and the amount of heterogeneity that can be tolerated in these recombination sequences can be empirically evaluated, for example, by evaluating integration efficiency of a targeting construct using an altered recombinase of the present invention (for exemplary methods of evaluating integration events, see, WO 00/11155, published Mar. 2, 2000).

Functional pseudo-sites can also be found empirically. For example, experiments performed in support of the present invention have shown that after co-transfection into human cells of a plasmid carrying φC31 attB and the neomycin resistance gene, along with a plasmid expressing the φC31 integrase, an elevated number of neomycin resistant colonies are obtained, compared to co-transfections in which either attB or the integrase gene were omitted. Most of these colonies reflected integration into native pseudo attP sites. Such sites are recovered, for example, by plasmid rescue and analyzed at the DNA sequence level, producing, for example, the DNA sequence of a pseudo attP site from the human genome. This empirical method for identification of pseudo-sites can be used, even if a detailed knowledge of the recombinase recognition sites and the nature of recombinase binding to them are unknown.

In some embodiments, the first vector recombination site of the target vector is a bacterial genomic recombination site (attB) or a phage genomic recombination site (attP) recognized by a first site-specific recombinase. In such embodiments, the genomic recombination site present in the genome of the target cell is a corresponding pseudo-recombination site. For example, where the first vector recombination site of the target vector is a bacterial genomic recombination site (attB), the genomic pseudo-recombination site present in the genome of the target cell is a pseudo-phage genomic recombination site (pseudo-attP). Likewise, where the first vector recombination site of the target vector is a phage genomic recombination site (attP), the genomic pseudo-recombination site present in the genome of the target cell is a pseudo-bacterial genomic recombination site (pseudo-attB).

Some unidirectional site-specific recombinases preferentially integrate into pseudo-bacterial recombination sites (e.g., pseudo-attB), rather than pseudo-phage recombination sites (e.g., pseudo-attP). In these cases, the target vector carries a phage recombination site (attP) and will integrate into pseudo-attB site. Examples of enzymes with this preference are phiBT1 integrase and A118 integrase. In such embodiments, the first vector recombination site of the target vector is an attP site and the genomic recombination site in the genome of the target cell is a pseudo-attB site. Other unidirectional, site-specific recombinases, such as φC31 and R4, prefer to integrate into pseudo-phage attachment sites (pseudo-attP sites) rather than pseudo-bacterial recombination sites (pseudo-attB sites), so the target vector carries an attB site and will integrate into a pseudo-attP site (Groth et al, 2000; Olivares, Hollis and Calos 2001). In such embodiments, the first vector recombination site of the target vector is an attB site and the genomic recombination site in the genome of the target cell is a pseudo-attP site.

Furthermore, in certain embodiments, the first vector recombination site of the target vector is a pseudo-recombination site and the genomic recombination site present in the genome of the target cell is a corresponding pseudo-recombination site recognized by a first site-specific recombinase. For example, where the vector recombination site of the target vector is a pseudo-bacterial genomic recombination site (pseudo-attB), the pseudo-recombination site present in the genome of the target cell is a pseudo-phage genomic recombination site (pseudo-attP). Likewise, where the first vector recombination site of the target vector is a pseudo-phage genomic recombination site (pseudo-attP), the pseudo-recombination site present in the genome of the target cell is a pseudo-bacterial genomic recombination site (pseudo-attB).

In some embodiments, the second vector recombination site of the target vector is a bacterial genomic recombination site (attB) or a phage genomic recombination site (attP) recognized by a second site-specific recombinase. In such embodiments, the donor recombination site on the donor vector is a corresponding recombination site. For example, in embodiments where the second vector recombination site of the target vector is a bacterial genomic recombination site (attB), the donor recombination site present on the donor vector is a phage genomic recombination site (attP). Likewise, where the second vector recombination site of the target vector is a phage genomic recombination site (attP), the donor recombination site present on the donor vector is a bacterial genomic recombination site (attB).

As noted above, the target vector includes a first portion of a first selectable marker adjacent to a 3′ side of the second vector recombination site and the donor vector includes a second portion of the first selectable marker adjacent to a 5′ side of the donor recombination site. In the presence of a second unidirectional site-specific recombinase the second vector recombination site on the target vector recombines with the donor recombination site present on the donor vector to generate a hybrid recombination site. As a result of the recombination, the first portion of the selectable marker on the target vector and second portion of the selectable marker on the donor vector are brought into close proximity to provide for a reconstituted functional first selectable marker. Therefore, selection using the first selection marker can be used to screen for successful recombination events between a target vector present in the genome of a target cell and donor vector having a polynucleotide encoding a protein of interest.

In one embodiment of the reconstituted first selectable marker gene the promoter is provided by the donor vector and a coding region for a selectable marker gene and polyadenylation signal is provided by the target vector. In another embodiment of the reconstituted selectable marker gene the donor vector may contain a promoter, an N-terminal part of the coding region, and the 5′ half of an intron, while the target vector may contain the 3′ half of an intron, the C-terminal part of the coding region, and a polyadenylation signal. In a further embodiment of the reconstituted selectable marker gene the donor vector may contain a promoter and the N-terminal part of the coding region while the target vector may contain the C-terminal part of the coding region and a polyadenylation signal. In still another embodiment, the donor vector includes a promoter and the target vector includes a promoter-less selectable marker. In all of these embodiments of the reconstituted selectable marker gene, the key feature is that the genetic elements present in the separate target and donor vectors are incapable of conferring drug resistance independent of one another. However when the donor vector is integrated into the target vector a complete functional gene expression cassette is assembled the cells which contain such a configuration will be resistant to the drug that is used to select for the presence of the reconstituted selectable marker gene.

Promoter and promoter-enhancer sequences are DNA sequences to which RNA polymerase binds and initiates transcription. The promoter determines the polarity of the transcript by specifying which strand will be transcribed. Bacterial promoters consist of consensus sequences, −35 and −10 nucleotides relative to the transcriptional start, which are bound by a specific sigma factor and RNA polymerase.

Eukaryotic promoters are more complex. Most eukaryotic promoters utilized in expression vectors are transcribed by RNA polymerase II. General transcription factors (GTFS) first bind specific sequences near the transcription start site and then recruit the binding of RNA polymerase II. In addition to these minimal promoter elements, small sequence elements are recognized specifically by modular DNA-binding, trans-activating proteins (e.g. AP-1, SP-1) that regulate the activity of a given promoter. Viral promoters serve the same function as bacterial or eukaryotic promoters and either require a promoter-specific RNA polymerase in trans (e.g., bacteriophage T7 RNA polymerase in bacteria) or recruit cellular factors and RNA polymerase II (in eukaryotic cells). Viral promoters (e.g., the SV40, RSV, and CMV promoters) may be preferred as they are generally particularly strong promoters.

Promoters may be, furthermore, either constitutive or regulatable. Constitutive promoters constantly express the gene of interest. In contrast, regulatable promoters (i.e., derepressible or inducible) express genes of interest only under certain conditions that can be controlled. Derepressible elements are DNA sequence elements which act in conjunction with promoters and bind repressors (e.g. lacO/lacIq repressor system in E. coli). Inducible elements are DNA sequence elements which act in conjunction with promoters and bind inducers (e.g. gal1/gal4 inducer system in yeast). In either case, transcription is virtually “shut off” until the promoter is derepressed or induced by alteration of a condition in the environment (e.g., addition of IPTG to the lacO/lacIq system or addition of galactose to the gal1/gal4 system), at which point transcription is “turned-on.”

Another type of regulated promoter is a “repressible” one in which a gene is expressed initially and can then be turned off by altering an environmental condition. In repressible systems transcription is constitutively on until the repressor binds a small regulatory molecule at which point transcription is “turned off”. An example of this type of promoter is the tetracycline/tetracycline repressor system. In this system when tetracycline binds to the tetracycline repressor, the repressor binds to a DNA element in the promoter and turns off gene expression.

Examples of constitutive prokaryotic promoters include the int promoter of bacteriophage λ, the bla promoter of the β-lactamase gene sequence of pBR322, the CAT promoter of the chloramphenicol acetyl transferase gene sequence of pPR325, and the like.

Examples of inducible prokaryotic promoters include the major right and left promoters of bacteriophage (P_{L} and P_{R}), the tip, recA, lacZ, AraC and gal promoters of E. coli, the α-amylase (Ulmanen Ett at., J. Bacteriol. 162:176-182, 1985) and the sigma-28-specific promoters of B. subtilis (Gilman et al., Gene sequence 32:11-20(1984)), the promoters of the bacteriophages of Bacillus (Gryczan, In: The Molecular Biology of the Bacilli, Academic Press, Inc., NY (1982)), Streptomyces promoters (Ward et at., Mol. Gen. Genet. 203:468-478, 1986), and the like. Exemplary prokaryotic promoters are reviewed by Glick (J. Ind. Microtiot. 1:277-282, 1987); Cenatiempo (Biochimie 68:505-516, 1986); and Gottesman (Ann. Rev. Genet. 18:415-442, 1984).

Exemplary constitutive eukaryotic promoters include, but are not limited to, the following: the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gal1 gene sequence promoter (Johnston et al., Proc. Natl. Acad. Sci. (USA) 79:6971-6975, 1982); Silver et al., Proc. Natl. Acad. Sci. (USA) 81:5951-59SS, 1984), the CMV promoter, the EF-1 promoter.

Examples of inducible eukaryotic promoters include, but are not limited to, the following: ecdysone-responsive promoters, the tetracycline-responsive promoter, promoters regulated by “dimerizers” that bring two parts of a transcription factor together, estrogen-responsive promoters, progesterone-responsive promoters, riboswitch-regulated promoters, antibiotic-regulated promoters, acetaldehyde-regulated promoters, and the like.

Some regulated promoters can mediate both repression and activation. For example, in the RheoSwitch system a protein (the RheoReceptor) binds to a DNA element (UAS, upstream activating sequence) in the promoter and mediates repression. However in the presence of certain ecdysone-like inducers another protein (the RheoActivator) will bind to the inducer. The inducer-bound RheoActivator is capable of binding to the DNA-bound RheoReceptor. The RheoReceptor/inducer/RheoActivator is then capable of actrivating gene expression.

Common selectable marker genes include those for resistance to antibiotics such as ampicillin, tetracycline, kanamycin, bleomycin, streptomycin, hygromycin, neomycin, puromycin, G418, bleomycin, blasticidin, Zeocin™, and the like. Selectable auxotrophic genes include, for example, hisD, that allows growth in histidine free media in the presence of histidinol.

A further element useful in an expression vector is an origin of replication. Replication origins are unique DNA segments that contain multiple short repeated sequences that are recognized by multimeric origin-binding proteins and that play a key role in assembling DNA replication enzymes at the origin site. Suitable origins of replication for use in expression vectors employed herein include E. coli oriC, ColE1 plasmid origin, 2μ, and ARS (both useful in yeast systems), sf1, SV40, EBV oriP (useful in eukaryotic systems, such as a mammalian system), and the like.

As noted above, the donor vector includes a multiple cloning site or polylinker. A multiple cloning site or polylinker is a synthetic DNA encoding a series of restriction endonuclease recognition sites inserted into a donor vector and allows for convenient cloning of polynucleotides encoding the protein of interest into the donor vector at a specific position.

Useful proteins that may be produced by the compositions and methods of the invention are, for example, enzymes that can be used for the production of nutrients and for performing enzymatic reactions in chemistry, or polypeptides which are useful and valuable as nutrients or for the treatment of human or animal diseases or for the prevention thereof, for example hormones, polypeptides with immunomodulatory activity, anti-viral and/or anti-tumor properties (e.g., maspin), antibodies, viral antigens, vaccines, clotting factors, enzyme inhibitors, foodstuffs, and the like. Other useful polypeptides that may be produced by the methods of the invention are, for example, those coding for hormones such as secretin, thymosin, relaxin, luteinizing hormone, parathyroid hormone, adrenocorticotropin, melanoycte-stimulating hormone, β-lipotropin, urogastrone or insulin, growth factors, such as epidermal growth factor, insulin-like growth factor (IGF), e.g. IGF-I and IGF-II, mast cell growth factor, nerve growth factor, glial cell line-derived neurotrophic factor (GDNF), or transforming growth factor (TGF), such as TGF-α or TGF-β (e.g. TGF-β1, β2 or β3), growth hormone, such as human or bovine growth hormones, interleukins, such as interleukin-1 or -2, human macrophage migration inhibitory factor (MIF), interferons, such as human α-interferon, for example interferon-αA, αB, αD or αF, α-interferon, γ-interferon or a hybrid interferon, for example an αA-αD- or an αB-αD-hybrid interferon, especially the hybrid interferon BDBB, protease inhibitors such as α₁-antitrypsin, SLPI, α₁-antichymotrypsin, C1 inhibitor, hepatitis virus antigens, such as hepatitis B virus surface or core antigen or hepatitis A virus antigen, or hepatitis nonA-nonB (i.e., hepatitis C) virus antigen, plasminogen activators, such as tissue plasminogen activator or urokinase, tumor necrosis factors (e.g., TNF-α or TNF-β), somatostatin, renin, β-endorphin, immunoglobulins, such as the light and/or heavy chains of immunoglobulin A, D, E, G, or M or human-mouse hybrid immunoglobulins, immunoglobulin binding factors, such as immunoglobulin E binding factor, e.g. sCD23 and the like, calcitonin, human calcitonin-related peptide, blood clotting factors, such as factor IX or VIIIc, erythropoietin, eglin, such as eglin C, desulphatohirudin, such as desulphatohirudin variant HV1, HV2 or PA, human superoxide dismutase, viral thymidine kinase, β-lactamase, glucose isomerase, transport proteins such as human plasma proteins, e.g., serum albumin and transferrin. Fusion proteins of the above may also be produced by the methods of the invention.

Furthermore, the levels of an expressed protein of interest can be increased by vector amplification (see Bebbington and Hentschel, “The use of vectors based on gene amplification for the expression of cloned genes in mammalian cells in “DNA cloning”, Vol. 3, Academic Press, New York, 1987). When a marker in the vector system expressing a protein is amplifiable, an increase in the level of an inhibitor of that marker, when present in the host cell culture, will increase the number of copies of the marker gene. Since the amplified region is associated with the protein-encoding gene, production of the protein of interest will concomitantly increase (Crouse et al., 1983, Mol. Cell. Biol., 3:257). An exemplary amplification system includes, but is not limited to, dihydrofolate reductase (DHFR), which confers resistance to its inhibitor methotrexate. Other suitable amplification systems include, but are not limited to, glutamine synthetase (and its inhibitor methionine sulfoximine), thymidine synthase (and its inhibitor 5-fluoro uridine), carbamyl-P-synthetase/aspartate transcarbamylase/dihydro-orotase (and its inhibitor N-(phosphonacetyl)-L-aspartate), ribonucleoside reductase (and its inhibitor hydroxyurea), ornithine decarboxylase (and its inhibitor difluoromethyl ornithine), adenosine deaminase (and its inhibitor deoxycoformycin), and the like.

Each of these systems requires the use of a cell line that is deficient in the marker gene that is amplified. For example use of the DHFR gene as an amplifiable gene uses a DHFR-deficient cell line, such as a DHFR-deficient CHO cell (e.g., DG44). Methods are available for isolating such marker gene-deficient cell lines. A gene amplification system that does not use marker gene-deficient cell lines is a system that uses the adeno-associated virus type 2 (AAV-2) rep protein and the rep protein binding site.

Most amplifiable marker genes may also be used as selectable marker genes. For example the presence of the DHFR gene can be selected in DHFR-deficient cells by using cell growth media that lacks glycine, thymidine, and hypoxanthine. The presence of the glutamine synthetase gene can be selected in glutamine synthetase-deficient cells by using media that lacks glutamine, and so on. In this manner one can ensure that the amplifiable marker gene is present in order to mediate gene amplification, especially prior to any gene amplification procedures.

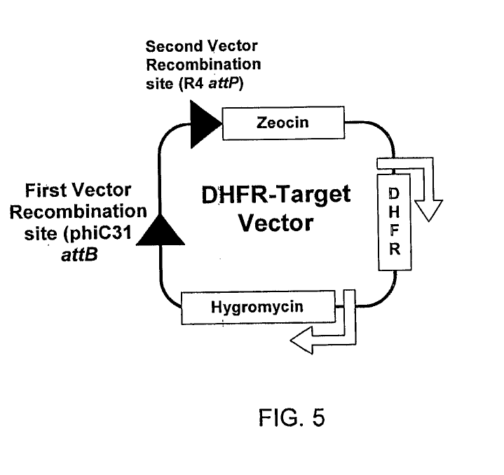

Accordingly, in certain embodiments, the target vector further includes a polynucleotide encoding the selectable and amplifiable marker gene DHFR. An exemplary target vector including DHFR is provided in FIG. 5. In such embodiments, the target vector that is integrated into the genome of the target cell is amplified using increasing concentrations of methotrexate. Since the target vector comprises a second site-specific recombinase site for integration of the donor vector, amplification of the target vector sequence in the genome of the target cell will result in amplification of the number of second site-specific recombinase sites present in the genome of the target cell. This provides a plurality of locations in which the donor vector can integrate.

In other embodiments, the donor expression vector is optionally integrated into the target-DHFR vector prior to exposure to increasing concentrations of methotrexate. In such embodiments, the gene encoding the protein of interest located on the donor expression vector will become closely linked (within 4,000 base pairs) to the DHFR gene located on the target-DHFR vector. As a result of the methotrexate exposure, the copy number of the gene encoding the protein of interest will be amplified by selection of cells in increasing concentrations of methotrexate.

In a traditional method of gene amplification, the DHFR gene is cotransfected with a protein expression vector in such excess (usually 100-fold) that it usually becomes linked to the protein expression vector but only after fragmentation and ligation of both vectors by cellular mechanisms. As opposed to a traditional method of gene amplification, this optional method provides the advantage of being able to control the arrangement, composition, and location of the DHFR gene relative to the protein expression gene prior to exposure to methotrexate. As a result this will provide a higher frequency of successful gene amplification and result in fewer unstable cell lines that do not express the gene of interest or loose expression of the gene of interest over time.

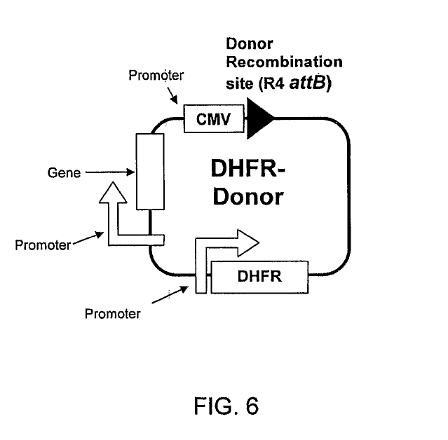

Alternatively, in other embodiments, the donor vector having the polynucleotide encoding the protein of interest further includes a polynucleotide encoding the selectable and amplifiable marker gene DHFR. An exemplary donor vector including DHFR is provided in FIG. 6. In such embodiments, the entire sequence that is integrated into the genome, including the polynucleotide encoding the protein of interest, is amplified using increasing concentrations of methotrexate.

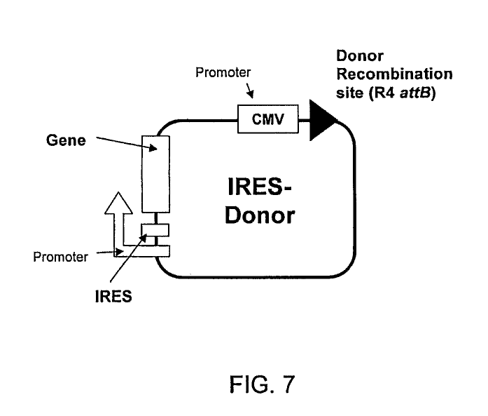

In certain embodiments, the donor vector further includes an internal ribosome entry site (IRES) positioned between the transcription start site and the translation initiation codon of the protein of interest. An exemplary donor vector including an IRES is provided in FIG. 7. Such vectors may allow for increased gene expression if they are translational enhancers or they can also allow for production of multiple proteins of interest from a single transcript, as long as an IRES is located 5′ to each coding region of interest.

The vectors described herein can be constructed utilizing methodologies known in the art of molecular biology (see, for example, Ausubel or Maniatis) in view of the teachings of the specification. An exemplary method of obtaining polynucleotides, including suitable regulatory sequences (e.g., promoters) is PCR. General procedures for PCR are taught in MacPherson et al., PCR: A PRACTICAL APPROACH, (IRL Press at Oxford University Press, (1991)). PCR conditions for each application reaction may be empirically determined. A number of parameters influence the success of a reaction. Among these parameters are annealing temperature and time, extension time, Mg²⁺ and ATP concentration, pH, and the relative concentration of primers, templates and deoxyribonucleotides. After amplification, the resulting fragments can be detected by agarose gel electrophoresis followed by visualization with ethidium bromide staining and ultraviolet illumination.

### Methods

The present invention also provides methods of generating a cell line that produces a protein of interest by site specifically integrating a polynucleotide encoding the protein of interest into the genome of a eukaryotic cell, such as a mammalian cell. In general the method involves first introducing a target vector as described herein into a eukaryotic cell by utilizing a first unidirectional site-specific recombinase and maintaining the cell under conditions sufficient for a recombination event mediated by the first unidirectional site-specific recombinase between the first vector recombination site and the genomic recombination site in order to site-specifically integrate the target vector into the genome of the cell. Successful integration events of the target vector mediated by the first unidirectional site-specific recombinase can be selected by using the selectable marker gene present on the target vector.

A donor vector comprising the polynucleotide encoding a protein of interest and a donor recombination site is then introduced into the target cell by utilizing a second unidirectional site-specific recombinase. The target cell is then maintained under conditions sufficient to allow for a recombination event mediated by the second unidirectional site-specific recombinase to occur. As a result, a recombination event between the donor recombination site and the second vector recombination site of the target vector allows for site-specific integration of the polynucleotide encoding a protein of interest into the genome of the cell. Successful integration events of the donor vector mediated by the second unidirectional site-specific recombinase can be selected by using a reconstituted first selectable marker gene. In one embodiment of the reconstituted first selectable marker gene the promoter is provided by the donor vector and a coding region for a selectable marker gene and polyadenylation signal is provided by the target vector. In another embodiment of the reconstituted selectable marker gene the donor vector may contain a promoter, an N-terminal part of the coding region, and the 5′ half of an intron, while the target vector may contain the 3′ half of an intron, the C-terminal part of the coding region, and a polyadenylation signal. In a further embodiment of the reconstituted selectable marker gene the donor vector may contain a promoter and the N-terminal part of the coding region while the target vector may contain the C-terminal part of the coding region and a polyadenylation signal. In still another embodiment, the donor vector includes a promoter and the target vector includes a promoter-less selectable marker. In all of these embodiments of the reconstituted selectable marker gene, the key feature is that the genetic elements present in the separate target and donor vectors are incapable of conferring drug resistance independent of one another. However when the donor vector is integrated into the target vector a complete functional gene expression cassette is assembled the cells which contain such a configuration will be resistant to the drug that is used to select for the presence of the reconstituted selectable marker gene.

In general, the unidirectional site-specific integrase interaction with the site-specific recombination sites produces a recombination product that does not contain a sequence that acts as an effective substrate for the unidirectional site-specific integrase. Thus, the integration event employed in the subject methods is unidirectional, with little or no detectable excision of the introduced nucleic acid mediated by the unidirectional site-specific integrase. This feature ensures greater stability of expression of proteins of interest compared to other integration systems than can be provided by a bidirectional site specific recombinase (e.g., the lox/cre integration system) or that contain directly repeated sequences (e.g., long terminal repeats) which may result in deletion of genes encoding proteins of interest (e.g., in retrovirus or lentivirus integration systems)

The vectors can be introduced into the host cell by any one of the standard means practiced by one with skill in the art to produce a cell line of the invention. The nucleic acid vectors can be delivered, for example, with cationic lipids (Goddard, et al, Gene Therapy, 4:1231-1236, 1997; Gorman, et al, Gene Therapy 4:983-992, 1997; Chadwick, et al, Gene Therapy 4:937-942, 1997; Gokhale, et al, Gene Therapy 4:1289-1299, 1997; Gao, and Huang, Gene Therapy 2:710-722, 1995, all of which are incorporated by reference herein), using viral vectors (Monahan, et al, Gene Therapy 4:40-49, 1997; Onodera, et al, Blood 91:30-36, 1998, all of which are incorporated by reference herein), by uptake of “naked DNA”, chemical means (e.g., calcium phosphate), electrophoretic means, and the like.

The first and second unidirectional site-specific recombinases used in the practice of the present invention can be introduced into the target cell before, concurrently with, or after the introduction of a target vector or a donor vector. The first and second unidirectional site-specific recombinases can be introduced in the form of the DNA encoding the unidirectional site-specific recombinase (Olivares, Hollis and Calos, Gene, 278:167-176 (2001); Thyagarajan et al. MCB 21:3926-3934 (2001)), or mRNA encoding the unidirectional site-specific recombinase (Groth et al. JMB 335:667-678 (2004); Hollis et al. Repr. Biol. Endocrin. 1:79 (2003)), or as the unidirectional site-specific recombinase protein.

Expression of the first and second unidirectional site-specific recombinases is typically desired to be transient. This is because long term expression of recombinases may promote recombination between pseudo att sites present at various locations in the genome. This would lead to chromsomal rearrangements and eventually to cell death. Accordingly, vectors and methods providing transient expression of the recombinase are preferred in the practice of the present invention. However, stable expression of the first and second unidirectional site-specific recombinases may be acceptable if it is regulated, for example, by placing the expression of the recombinase under the control of a regulatable promoter (i.e., a promoter whose expression can be selectively induced or repressed).

Introduction of the first and second unidirectional site-specific recombinases as proteins has several advantages. The protein has a short half-life, so exposure of the cells to the unidirectional site-specific recombinase is limited in time. Furthermore, there is no chance of integration of the unidirectional site-specific recombinase gene into the genome. Limitations with transcription or translation of unidirectional site-specific recombinase are avoided, and the reaction kinetics may be more rapid. Introduction of protein into cells is generally less toxic than introduction of DNA. Therefore, introduction of a phage unidirectional site-specific recombinase into the eukaryotic cells as a protein may be preferable.

Proteins such as phage unidirectional site-specific recombinase can be introduced into cells by many means, including electroporation, peptide transporters (Siprashvili, Reuter and Khavari, Mol. Ther., 9:721-728 (2004)), or attachment of protein transduction domains, such as those derived from the Herpes Simplex Virus VP22 protein, antennapedia-derived peptides, various arginine-rich peptides, or the Human Immunodeficiency Virus tat protein. DNA or RNA encoding a unidirectional site-specific recombinase can also be introduced into cells by many means, including electroporation, complexing with chemical agents, such as electrostatic interaction with transporter molecules, or endocytosis.

Cells suitable for use with the subject methods of the present invention are generally any higher eukaryotic cell, such as mammalian cells and yeast cells. In some embodiments, the cells are an easily manipulated, easily cultured mammalian cell line. In other embodiments, the cells are an easily manipulated, easily cultured yeast cell line. Suitable cells that are capable of expressing recombinant DNA molecules, include, but are not limited to, mammalian cells such as a rodent cell, such as Chinese hamster ovary (CHO) cells, BHK cells, mouse cells including SP2/0 cells and NS-0 myeloma cells, primate cells such as COS and Vero cells, MDCK cells, BRL 3A cells, hybridomas, tumor cells, immortalized primary cells, human cells such as W138, HepG2, HeLa, HEK293, HT1080, or PER.C6™, and the like.

In some embodiments, the cell is a PER.C6™ cell. In other embodiments, the cell is a CHO cell or a dihydrofolate reductase-deficient cell such as DG44 cells. CHO cells have become a routine and convenient production system for the generation of biopharmaceutical proteins and proteins for diagnostic purposes. A number of characteristics make CHO cells suitable as a host cell. The production levels that can be reached in CHO cells are extremely high. The cell line provides a safe production system, which can be free of infectious agents and infections viral particles. CHO cells have been extensively characterized, are capable of growth in suspension until reaching high densities in bioreactors, using serum-free culture media, and a DHFR-deficient mutant of CHO cells (DG-44 clone. Urlaub et al., Cell. 33(2):405-12 (1983)) has been developed to obtain an easy selection and amplification system by introducing an exogenous DHFR gene, selecting for its presence, and thereafter performing a well-controlled, stepwise amplification of the DHFR gene and any linked genes of interest using increasing concentrations of methotrexate.

### Cell Lines

The present invention also provides cell lines generated by integrating the target vector described above into the genomic recombination site of the target cell. Accordingly, the subject cells have a genomically integrated polynucleotide cassette comprising a first hybrid recombination site and a second hybrid recombination site flanking a vector recombination site that recombines with a donor recombination site in the presence of a unidirectional site-specific recombinase; a promoter-less first selectable marker adjacent to the vector recombination site's 3′ end; and a second selectable marker that is different from the first selectable marker.

In some embodiments, the vector recombination site is a bacterial genomic recombination site (attB) or a phage genomic recombination site (attP). In some embodiments, the donor recombination site is a bacterial genomic recombination site (attB) or a phage genomic recombination site (attP). In some embodiments, the unidirectional site-specific recombinase is a φC31 phage recombinase, a TP901-1 phage recombinase, or an R4 phage recombinase. In some embodiments, the mammalian cell is a rodent cell. In other embodiments, the mammalian cell is a CHO cell. In yet other embodiments, the mammalian cell is a PER.C6™ cell.

### Kits

Also provided by the subject invention are kits for practicing the subject methods, as described above. In certain embodiments, the subject kits at least include one or more of, and usually all of a target vector and a donor vector as described above. In some embodiments, the kits further include a first and second unidirectional site-specific recombinase component, where the recombinase component can be provided in any suitable form (e.g., as a protein formulated for introduction into a target cell or in a recombinase vector which provides for expression of the desired recombinase following introduction into the target cell).

In other embodiments, the subject kits at least include one or more of, and usually all of an isolated cell line having an integrated target vector and a donor vector as described above. In some embodiments, the kits further include a first and second unidirectional site-specific recombinase component, where the recombinase component can be provided in any suitable form (e.g., as a protein formulated for introduction into a target cell or in a recombinase vector which provides for expression of the desired recombinase following introduction into the target cell).

Other optional components of the kit include restriction enzymes, control plasmids, buffers, materials for introduction of vectors into cells, etc. The various components of the kit may be present in separate containers or certain compatible components may be precombined into a single container, as desired.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Construction of Target and Donor Vectors

High-level expression of transgenes has been difficult to achieve consistently in CHO cells and other mammalian cell lines because of the random nature of integration and associated chromosomal context effects upon the integrated transgene. Using site-specific integrases from phages φC31 and R4, site specific integration vectors can be generated in order to provide for site specific integration of expression cassettes encoding a gene of interest in the genome of a mammalian cell.

The φC31 and R4 integration systems remove many of the limitations of random integration by providing integration into a relatively small number of locations in the genome that are also characterized by robust gene expression. Integration of transgenes with the φC31 or R4 integrase affords a facile method to generate mammalian cell lines that display stable, high-level expression of the introduced gene. Use of phage integrases to generate production cell lines thus reduces the time and effort required in isolating clones suitable for protein production. Therefore, since integration is thought to most favorably occur in places on chromosomes with open chromatin or reduced methylation, such locations will also be most favorable for high level, sustained gene expression.

### Target Vector

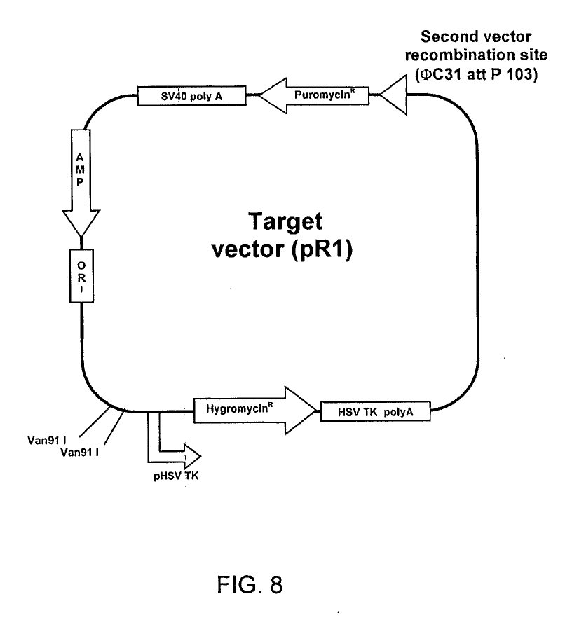

A schematic map of an exemplary target vector for use in introducing a site specific integrase attachment site in the genome of cell line is provided in FIG. 1 and FIG. 8. In general the target vector will include a first attachment site for a first site-specific integrates and a second attachment site for a second site-specific integrase (e.g., an altered, site-specific integrase with a higher integration efficiency), wherein the first and second site-specific integrases are different. The target vectors may also include further elements, such as a bacterial selectable marker (e.g., β-lactamase encoding resistance to ampicillin) that provides for selection of prokaryotic cells containing the vectors. In addition, the vector may also include a mammalian cell specific selectable marker (e.g., a gene encoding hygromycin B phosphotransferase encoding resistance to the drug hygromycin) for selecting mammalian cells that have the target vector successfully integrated into the genome, and an origin for vector replication (e.g., the ColE1 origin of DNA replication) in bacterial cells, such as E. coli.

As shown in FIG. 12 and FIG. 13, the target vector will be used for introducing a nucleic acid sequence encoding the φC31 attP 103 site into the genome of cells, such as mammalian cells. Once integrated, this φC31 attP 103 site will be used for site specifically integrating a donor plasmid that includes an expression cassette for a gene of interest and a nucleic acid sequence encoding the φC31 attB 285 AAA site. The initial target vector includes the nucleic acid sequences for two different att sites for two different site specific integrases. In particular, the target vector will include a nucleic acid sequence encoding the R4 attB 295 site. The R4 attB 295 site mediates integration of the target vector into R4 pseudo attP (R4 Ψ attP) sites in the mammalian cell genome. There are estimated to be about 100 R4 Ψ attP sites in a typical mammalian genome. The target vector will also include a nucleic acid sequence encoding a φC31 attP 103 site. The φC31 attP 103 site serves as a target site for integration of the donor vector that includes an expression cassette designed to direct expression of genes of interest.

The order of integration chosen here, namely R4 integrase-mediated integration followed by φC31 mutant integrase-mediated integration, is chosen for two reasons. R4 integrase-mediated integration was chosen as the first step, instead of φC31 integrase-mediated integration, because there are fewer R4 Ψ attP sites compared to φC31 Ψ attP sites in mammalian genomes. Therefore the number of sites at which integration will occur is less and fewer clones will need to be screened to identify those with the highest levels of protein expression. φC31 mutant integrase-mediated integration is chosen as the second step because once first integration sites are identified that result in high level protein expression after donor vector integration, it is desirable to have integration of the donor vector be as efficient as possible. Hence a mutant φC31 integrase will be used. Mutants of φC31 integrase have been identified that result in up to 75% of integration events occurring at the wild type att P site contained on an integrated vector (such as that contained on the target vector), while the remaining 25% occur at a variety of φC31%Ψ attP sites. There are estimated to be about 370 (range=202-764 with a 95% confidence interval) φC31 Ψ attP sites in human cells, such as 293, D407, and HepG2 cells (Chalberg, et al., 2006). The site at which integration most frequently occurs can vary between different cells but is typically <5-10% of the total number of sites that can serve as integration sites. If a less efficient integrase is used that had a lower degree of selectivity for wild type attP sites over pseudo attP sites, then more integration would occur at φC31 Ψ attP sites rather than at the desired wild type attP site in the integrated target vector.

In addition, the target vector also includes a nucleic acid sequence encoding the selectable marker hygromycin, which is used to select hygromycin resistant-clones that have a genomically integrated target vector. The target vector has a first portion of a (e.g., promoter-less) puromycin coding region and a SV40 poly A signal downstream of the nucleic acid sequence encoding the φC31 attP 103 site. Upon integration of the donor vector, a SV40 promoter is introduced upstream of the puromycin gene, thereby reconstituting a complete gene expression cassette capable of providing expression of the selectable marker. Therefore, the reconstituted puromycin selectable marker can be used to efficiently select for successful recombination events between a φC31 attB site (e.g., a φC31 attB 285 AAA site) on the donor vector and a φC31 attP site (e.g. a φC31 attP 103 site) present on the target vector.

A weaker promoter (e.g., SV40) and more toxic drug for selection (e.g., puromycin) are chosen as opposed to stronger promoters (e.g., CMV) and weaker drugs for selection (e.g., G418) in order to provide a stronger selection for the desired donor vector integration event. This step, the integration of the donor vector into the integrated target vector, is the key step of the invention that allows a site specific integration of the donor vector, which contains expression cassettes for genes of interest. However, it is possible that a wide variety of promoters (without coding regions) on the donor vector may work as efficiently. In addition a wide variety of coding regions for drug resistance genes (without promoters) present on the target vector may also work as efficiently. The examples given here, using an SV40 promoter and a puromycin coding region, are not meant to be exclusive.

In a similar manner a relatively weak promoter (herpes simplex virus thymidine kinase) is used to drive expression of the drug resistance marker (hygromycin) on the target vector. It has been reported by some that weaker expression of a co-selected marker can result in higher expression of linked genes of interest.

Construction of Target Vector

To construct the target vector (pR1; FIG. 8) the following steps were performed. The sequence of the pR1 vector is provided in FIGS. 33A-33B. A 295 bp fragment containing the R4 attB site (R4 attB 295) was amplified by PCR from rehydrated Streptomyces parvulus cells (ATCC 12434) using primers 5′-CGTGGGGACGCCGTACAG-3′ (SEQ ID NO:01) and 5′-CCCGGTCAACATCCAGTACACCT-3′ (SEQ ID NO:02) as described by Olivares et al., 2001 and cloned into pCR2.1-TOPO (Invitrogen) to make pTA-R4attB. R4 attB 295 was isolated from pTA-R4attB by digestion with EcoRI. This fragment was blunt-ended by filling in the ends with Klenow DNA polymerase and then ligated into pTK-Hyg (TaKaRa Clontech) at the Hind III site, which had also been blunt-ended by filling in the ends with Klenow DNA polymerase to make the vector pTK-R4B. DNA sequencing was used to confirm pTK-R4B had the correct sequence and also that the R4 attB 295 site was in the orientation shown in FIG. 8, namely that the right side of the R4 attB core recombination site (indicated by the narrow point of the triangle) was closest to the hygromycin resistance cassette.

Two polymerase chain reactions were done to amplify the φC31 attP 103 and the puromycin resistance coding region separately. Then they were fused together precisely using a third PCR. The PCR conditions were 95° C. for 1 minute to denature, 60° C. for 15 seconds to anneal, and 72° C. for 45 seconds to polymerize. The reactions were done with a proofreading enzyme (Pfu Ultra) that generates blunt-ended PCR products.

A 103 bp region of the φC31 attP site (φC31 attP 103) which contains sequences known to encode a functional attP site was amplified from pTA-attP (described by Olivares et al., 2001) using primers C31-attP-1 (5′-AAAAAAGAATTCGTACTGACGGACACACCGAAGCCCC-3′ (SEQ ID NO:03) and C31-attP-2 (5′-CACGGTAGGCTTGTACTCGGTCATGGTGGCGACCCTACGCCCCCAACTG-3′) (SEQ ID NO:04) resulting in a 186 bp product. The 5′ end of primer C31-attP-2 has 24 bases from 5′ end of puromycin resistance ORF.

The puromycin resistance coding region along with a polyadenylation signal from SV40 was amplified by PCR from pPUR (TaKaRa Clontech) using primers Puro1 (5′-CAGTTGGGGGCGTAGGGTCGCCACCATGACCGAGTACAAGCCCACGGT G-3′) (SEQ ID NO:05) and SV40polyA (5′-AAAAAACCTTTCGTCTTCAGACATGATAAGATACATTGATGAGTTTGG-3′) (SEQ ID NO:06) resulting in a 1001 bp product. The 5′ end of primer Puro1 had 24 bases from 3′ end of φC31 attP and the 3′ end of SV40polyA has a Bbs I restriction enzyme recognition site. The PCR conditions for the first 10 cycles were 95° C. for 1 minute to denature, 47° C. for 30 seconds to anneal, and 72° C. for 75 seconds to polymerize. The PCR conditions for the next 15 cycles were 95° C. for 1 minute to denature, 60° C. for 30 seconds to anneal, and 72° C. for 75 seconds to polymerize. The reactions were done with a proofreading enzyme (Pfu Ultra) that generates blunt-ended PCR products.

To fuse the DNA containing the φC31 attP 103 to the DNA containing the puromycin resistance coding region and SV40 polyadenylation signal the products of those separate PCRs were mixed in an equimolar ratio and amplified by PCR with primers C31-attP-1 and SV40 polyA to produce a 1138 bp product. The PCR conditions were 95° C. for 30 seconds to denature, 60° C. for 20 seconds to anneal, and 72° C. for 90 seconds to polymerize. The reactions were done with a proofreading enzyme (Pfu Ultra) that generates blunt-ended PCR products.

The 1138 bp PCR product containing φC31 attP 103, the puromycin resistance open reading frame, and the SV40 polyadenylation signal was digested with Bbs I and cloned into pTK-R4B which was digested with Swa I and Bbs I. This produced the target vector pR1. The sequences and proper orientation of φC31 attP 103, the puromycin resistance open reading frame, and the SV40 polyadenylation signal in pR1 were confirmed by DNA sequencing.

A key feature of the design of the φC31 attP 103-puromycin coding region fusion is diagrammed in FIG. 14. The 221 base pair long φC31 attP 221 site that is present in pTA-attP has an ATG that would end up being upstream of the puromycin coding region once the donor vector is integrated into the target vector, to create a φC31 attL site. Usually ATG sequences (potential translation initiation sites) that are upstream of legitimate coding regions are detrimental to gene expression. Therefore, in the PCR product that fuses φC31 attP 103 to the puromycin coding region, that ATG was made the start codon of the puromycin coding region. In addition, 2 bases prior to that ATG were changed to create a more optimal, consensus translation start (Kozak) sequence (GCCACC). As shown in FIG. 14 these changes are at least eighteen bases 3′ to the minimal, but fully functional, φC31 attP site identified by Groth et al., 2000. Therefore they should not affect the ability of the φC31 attB 285 AAA site in the donor vector to integrate into the φC31 attP 103 site in the target vector. After integration of the donor vector into the target vector the 88 base long φC31 attL site (q C31 attL 88) is located in the 5′ untranslated region, immediately before the puromycin coding region. Preceding φC31 attL 88 may be 57, 62, or 74 bases derived from the SV40 early promoter 5′ untranslated region (transcription directed by the SV40 early promoter begins at 3 different sites).

### Donor Vector

A schematic of an exemplary donor expression vector is provided in FIGS. 2 and 10. The exemplary donor expression vector contains a nucleic acid sequence encoding the φC31 attB 285 AAA site and a nucleic acid expression cassette encoding genes of interest, such as a cassette encoding the heavy and light chains of a human antibody. The donor vector also contains a SV40 promoter upstream of the nucleic acid sequence encoding the φC31 attB 285 AAA site. Upon integration of the donor vector into the previously integrated target vector, which is mediated by site specific recombination between the φC31 attB 285 AAA present on the donor vector and the φC31 attP 103 present in the target vector, the SV40 promoter will drive the expression of the puromycin gene (FIG. 13). Therefore, the reconstituted puromycin resistance gene can be used to select for cell clones that have integrated the genes on the donor vector for expressing proteins of interest.

This selection step is critical for achieving a high efficiency method because the φC31 attB 285 AAA site on the donor vector can also integrate into φC31 Ψ attP sites found at an estimated 370 chromsomal positions (Chalberg, et al., 2006). However all exemplary donor expression vectors that integrate into φC31 Ψ attP sites will contain only the SV40 promoter and will not reconstitute a functional puromycin resistance gene. Some puromycin resistant cells also result when integrase alone is expressed in an attP target vector clone (i.e., in the absence of a donor expression vector). Without being held to theory, the mechanism by which this occurs may involve recombination of Ψ attB sites that are near a cellular promoter with the attP 103 site in the target vector. Transfection of attP cell lines with a selectable donor expression vector and a second integrase expression vector addresses this concern because cells with no expression vector will not be resistant to the complete selectable drug resistance gene on the selectable donor expression vector. In addition, if necessary, desirable integration of donor vectors into chromosomal target vectors can easily be distinguished from undesirable random integration or integration of donor vectors into φC31 Ψ attP sites as described below in the section “Methods for cell line characterization”.

Construction of Donor Expression Vector

The donor expression vector (pD1-DTX-1) is based on pcDNA3002neo described by Jones et al., 2003. pcDNA3002neo is based on pcDNA3 (Invitrogen, Inc.). pcDNA3002neo contains two CMV promoters followed by two bovine growth hormone polyadenylation signals for expression of proteins in mammalian cells. pcDNA3002neo also includes a ColE1 origin and ampicillin resistance gene for maintenance and selection in E. coli. Finally, pcDNA3002neo vector has a G418 resistance gene expressed using an SV40 promoter and an SV40 polyadenylation signal. The sequence of the pD1-DTX-1 vector is provided in FIGS. 34A-34C.

To construct pD1-DTX-1, six inserts were cloned into pcDNA3002neo that contain 1) a polylinker with recognition sites for three restriction enzymes that cut within eight base pair long recognition sequences, 2) the φC31 attB 285 AAA region, 3) a first signal sequence that mediates secretion of proteins such as the heavy chain of a human antibody and contains a unique restriction site, 4) a second signal sequence that mediates secretion of proteins such as the light chain of a human antibody and contains another unique restriction site, 5) a coding region for a first protein such as the heavy chain of a human antibody specific for diphtheria toxin, and 6) a coding region for a second protein, such as the light chain of a human antibody specific for diphtheria toxin.

pcDNA3002neo lacks useful polylinkers after one of its CMV promoters. Therefore, as a first step to creating the donor vector pD1, a polylinker with three rarely occurring restriction sites was inserted. Two synthetic oligonucleotides (BamBst-A and BamBst-B) were annealed. The sequence of BamBst-A is: 5′-GATCCAAAAAATTAATTAAAAAAAACACCGGCGAAAAAAGCGATCGCA AAAAACCAGTGTG-3′ (SEQ ID NO:07). The sequence of BamBst-B is: 5′-CTGGTTTTTTGCGATCGCTTTTTTCGCCGGTGTTTTTTTTAATTAATTTTT TG-3′ (SEQ ID NO:08). When BamBst-A and BamBst-B are annealed they will contain Bam HI and Bst XI complementary sequences at their 5′ and 3′ ends, respectively, to allow ligation to Bam HI/Bst XI-digested pcDNA3002neo. The sequences will also include (in order from 5′ to 3′) restriction enzyme recognition sites for Pac I, SgrA I, and AsiS I. Spacer sequences of 6 adenosines separate each restriction site to allow efficient digestion at two adjacent sites, if needed. The two synthetic oligonucleotides were annealed as-is (i.e., unphosphorylated). pcDNA3002neo was digested with Bam HI at 37° C. and then with Bst XI at 55° C. The digested vector was ligated to the annealed polylinker and the ligation was transformed into XL-10 Gold (Stratagene) E. coli cells. The resulting vector was called pHPC-1.

A critical sequence element in the donor vector pD1 is the φC31 attB 285 AAA site. The φC31 attB 285 AAA site was amplified by PCR from the vector pT A-attB described by Olivares, et al, 2001. The 5′ primer was called C31attB-5′ and has a sequence of 5′-GTCGACGAAATAGGTCACGGTCTC-3′ (SEQ ID NO:09). The 3′ primer was called C31attB-3′ and has a sequence of 5′-TACGTCGACATGCCCGCCGTGACC-3′ (SEQ ID NO:10). The PCR conditions were denaturation at 95° C. for 1 minute, annealing at 60° C. for 15 seconds, and extension at 72° C. for 30 seconds using the Pfu Ultra polymerase (Stratagene). The concentration of other reaction components was the same as that of a standard PCR (e.g., 200 μM dNTPs, 1 μM each primer, 1.5 mM MgCl₂).

The 5′ primer changed an ATG sequence at the 5′ end of the φC31 attB site in pTA-attB to an AAA sequence. The reason for this is similar to that described above for the φC31 attP 103 site and is diagrammed in FIG. 14. The 5′ end of the φC31 attB 285 site that is present in pTA-attP has an ATG that would end up being upstream of the puromycin coding region once the donor vector is integrated into the target vector, to create a φC31 attL 88 site. Usually ATG sequences (potential translation initiation sites) that are upstream of legitimate coding regions are detrimental to gene expression. Therefore, the ATG at the 5′ end of φC31 attB was changed to AAA. All one base variants of AUG have been found to function as alternate translation initiation codons. However no two base variants have been shown to function as alternate translation initiation codons. Therefore in order to prevent the 5′ ATG in φC31 attB from being used as a translation initiation codon, but at the same time introduce a minimal number of changes to the sequence of φC31 attB, the ATG was changed to AAA. Since this ATG is near the 5′ end of the φC31 attB region contained in pTA-attB it was most convenient to incorporate the ATG to AAA change into the primer used to PCR the φC31 attB sequence from pTA-attB.

Amplification of pTA-attB by PCR with primers C31 attB-5′ and C31 attB-3′ resulted in a 285 base pair long product called φC31 attB 285 AAA. pHPC-1 was digested with Sma I and Bst Z17 I to produce 1130 bp and 5718 bp fragments. The φC31attB 286 AAA PCR product was ligated to the 5718 bp fragment. This produced a plasmid called pHPC-2. The plasmid with the φC31 attB 286 AAA sequence in an orientation such that the left side of attB was next to the SV40 promoter was called pHPC-2 (+) while the plasmid with the φC31 attB 286 AAA sequence in the opposite orientation was called pHPC-2 (−).

pHPC2(+) and pHPC-2(−) are useful as a vectors for integrating and expressing genes that encode proteins that are not secreted. However, to secrete proteins such as antibodies, hemophilic factors, growth factors, serum factors, or soluble receptors, a donor vector that contains a signal sequence for secretion would be desirable. Therefore a signal sequence (HAVT20; Boel et al., J Immunol Methods. 2000 May 26; 239(1-2):153-66) from a human T-cell receptor alpha chain was modified to have unique restriction sites. One version with a unique Pml I site was inserted at one of the two polylinkers in pHPC2(+) and another version with a unique PspX I site was inserted at the other polylinker in pHPC2(+). Neither version changed the amino acid sequence of the HAVT20 signal sequence and the changes also utilized frequently used human codons. Both the Pml I and the PspX I sites occur just before the signal sequence cleavage site. Therefore, a precise fusion between the cleavage site in the HAVT20 signal sequence and the coding region of a protein of interest is easily achieved by designing the appropriate PCR primers to amplify the coding regions of the genes of interest. Alternatively, it is possible to excise the HAVT20 signal sequence (e.g., using BamH I/Pac I at one cloning site and Asc I/Not I at the other cloning site) and insert other signal sequences. Those sequences could be heterologous (e.g., the IL-2 signal sequence) or homologous (e.g., a human IgG1 signal sequence).

To insert one HAVT20 signal sequence into pHPC-2(+) a duplex DNA encoding a Bam HI site at the 5′ end, an optimal consensus Kozak sequence, the HAVT20 signal sequence with a Pml I site, and a Pac I site at the 3′ end was generated by annealing 2 oligonucleotides: HAVT20-L-top (5′-CGCGCCACCATGGCATGCCCTGGCTTCCTGTGGGCACTTGTGATCTCCA CCTGCCTCGAGTTTTCCATGGCTCG-3′) (SEQ ID NO:11) and HAVT20-L-bot (3′-GGTGGTACCGTACGGGACCGAAGGACACCCGTGAACACTAGAGGTGGA CGGAGCTCAAAAGGTACCGAGC-5′) (SEQ ID NO:12). This annealed cassette was ligated to pHPC2(+) that was digested with Bam HI and Pac I. The resulting plasmid was called pHPC-3.

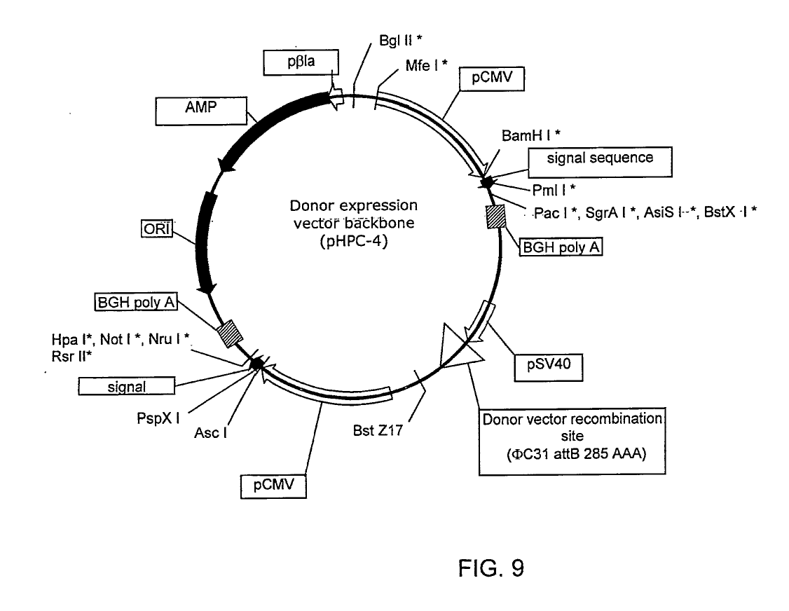

To insert a second HAVT20 signal sequence into pHPC-3 a duplex DNA encoding an Asc I site at the 5′ end, an optimal consensus Kozak sequence, the HAVT20 signal sequence with a PspX I site, and a blunt 3′ end was generated by annealing 2 oligonucleotides: HAVT20-H-top (5′-GATCCGCCACCATGGCATGCCCTGGCTTCCTGTGGGCACTTGTGATCTCC ACGTGTCTTGAATTTTCCATGGCTTTAAT-3′) (SEQ ID NO:13) and HAVT20-H-bot (3′-GCGGTGGTACCGTACGGGACCGAAGGACACCCGTGAACACTAGAGGTG CACAGAACTTAAAAGGTACCGAAAT-5′) (SEQ ID NO:14). This annealed cassette was ligated to pHPC3 that was digested with Asc I and Eco RV. The resulting plasmid is a donor expression vector backbone that may be used for, among other things, readily exchanging various gene expression elements, such as promoters. This donor expression vector backbone was called pHPC-4 (FIG. 9).

To isolate human IgG genes, EBV-transformed human B-cell lines that secrete antibodies which bind diphtheria toxin were derived as described by Traggiai, et al., 2004. One antibody with high affinity was subtyped and found to have a human IgG1 heavy chain and a kappa light chain. RNA was prepared from the cells producing this antibody and used in RT-PCR reactions to generate cDNAs encoding the heavy and light chain antibody genes. The primers used for amplification were similar to those described by Marks, et al. (Transplantation, 1991 August; 52(2):340-5), Sblattero, et al. (Immunotechnology, 1998 January; 3(4):271-8), and Yamanaka, et al. (J Biochem (Tokyo), 1995 June; 117(6): 1218-27) except that the ends had the appropriate restriction sites to allow subcloning. The light chain cDNA was cloned into the Not I/Xba I site of pBK-CMV (Stratagene) to create pBK-CMV-DTX-L. The heavy chain cDNA was cloned into the Hind III/Sal I site of pBK-CMV-DTX-L to create pABMC103. The cDNAs were sequenced and their identity as a human IgG1κ was confirmed.

To subclone the anti-diphtheria toxin antibody genes into pHPC-4 the entire heavy chain gene was amplified by PCR with primers 5′-AAAAAACACGTGTCTTGAATTTTCCATGGCTGAAGTGCAGCTGGTGGAG TCTGGG-3′ (SEQ ID NO:15) and 5′-AAAAAATTAATTAATTATTTACCCGGAGACAGGGAGAG-3′ (SEQ ID NO:16) using pABMC103 as a template. The resulting heavy chain PCR product was digested with BbrP I (isoschizomer of Pml I) and Pac I and cloned into pHPC-4 that was digested with BbrP I and Pac Ito create pHPC4-DTX-H. The entire light chain gene was amplified with primers 5′-AAAACCTCGAGTTTTCCATGGCTGAAACGACACTCACGCAGTCTCCAG3′ (SEQ ID NO:17) and 5′-AAAAAAGCGGCCGCTTAACACTCTCCCCTGTTGAAGCTCTTTG-3′ (SEQ ID NO:18) using pABMC103 as a template. The resulting light chain PCR product was digested with PspX I and Not I and cloned into pHPC4-DTX-H that was digested with PspX I and Not Ito create pD1-DTX-1. The sequences of both antibody chain genes were confirmed for both strands.

pHPC-2, pHPC-4, and pD1-DTX-1 can be subcloning vectors and expression vectors. Although the sequences of each of the two the CMV promoters, HAVT20 signal sequences, and bovine growth hormone polyadenylation signals are almost identical they are separated by polylinkers that are different in sequence. Therefore specific sequencing primers have been designed that are capable of sequencing genes inserted in each expression cassette. For example the primer 5′-GCTTGGTACCGAGCTCGGATCC-3′ (SEQ ID NO:19) can be used to sequence antibody variable regions inserted after the Pml I site of one signal sequence and the primer 5′-GAAGCTTGGTACCGGTGAATTCGG-3′ (SEQ ID NO:20) can be used to sequence antibody variable regions inserted after the PspX I site of the other signal sequence. Therefore, there is no need to clone genes of interest into other vectors for sequencing prior to cloning them into pHPC-2, pHPC-4 or pD1-DTX-1 for expression.

In addition, every element in pHPC-4 or pD1-DTX-1 is flanked by unique restriction sites such that any element (e.g., promoter, signal sequence, variable antibody chain, constant antibody chain, coding region, polyadenylation site, φC31 attB site) can easily be excised and replaced with other similar elements.

For example the heavy chain variable region can be exchanged by digesting pD1-DTX-1 with Pml I/Xho I and replacing the anti-diphtheria toxin antibody heavy chain variable region with other heavy chain variable regions. The light chain variable region can be exchanged by digesting pD1-DTX-1 with PspX I/BsiW I and replacing the anti-diphtheria toxin antibody light chain variable region with other light chain variable regions.

Similarly the IgG1 heavy chain constant region can be exchanged for those from other antibody subtypes (e.g., IgG2, IgG3, IgG4) or other immunoglobulin classes (e.g., IgA1, IgA2, IgD, IgE, or IgM) by exchanging an Apa I/Pac I restriction fragment. The kappa light chain constant region in pD1-DTX1 can be exchanged for a lambda kappa light chain constant region by exchanging a BsiW I/Not I restriction fragment.

One CMV promoter can be replaced with another promoter by exchanging a Mfe I/BamH I restriction fragment and the other CMV promoter can be replaced by exchanging a BstZ17 I/Asc I restriction fragment. One HAVT20 signal sequence can be replaced by exchanging a BamH I/Pml I restriction fragment and the other can be replaced by exchanging a Asc I/PspX I restriction fragment. One bovine growth hormone polyadenylation signal can be replaced by exchanging a AsiS I/NgoM IV restriction fragment and the other can be replaced by exchanging a Cla I/Pci I restriction fragment. The φC31 attB site can be replaced with an attB site recognized by another site-specific serine integrase by exchanging a Stu I/BstZ17 I restriction fragment.

Construction of Target-DHFR Vector

The target-DHFR vector (pR1-DHFR) was constructed by cloning a mouse DHFR expression cassette consisting of the SV40 promoter, a mouse DHFR coding region, the 3′ UTR of the mouse DHFR cDNA, and the Moloney murine leukemia virus (MLV) polyadenylation signal into the target vector pR1. The sequence of the pR1-DHFR vector is provided in FIGS. 35A-35C.

A 1,074 base pair DNA fragment from pSV2dhfr (American Type Culture Collection) containing the SV40 promoter, a mouse DHFR coding region, and part of the 3′ UTR of the mouse DHFR cDNA was amplified by PCR using primers 5′-CGAATCAGCACGGGGTGGCGCGCCCTGTGGAATGTGTGTCAGTTAGG-3′ (SEQ ID NO:21) and 5′-CGAATCAGCACGAAGTGCACCGGTGTTTAAACTTAATTAAAGATCTAAA GCCAGCAAAAGTCCCATGGT-3′ (SEQ ID NO:22). Conditions used for PCR were 95° C. for 30 seconds, 60° C. for 30 seconds, 72° C. for 90 seconds for 10 cycles, then 95° C. for 30 seconds and 72° C. for 90 seconds for 15 cycles using Pfu polymerase. The PCR product was then cloned into pCR-Blunt II-TOPO (Invitrogen), then digested with Dra III, and a fragment of 1050 base pairs was isolated and gel purified. pR1 was digested with Van91 I (isoschizomer of PflM I) and purified using a Qiagen PCR cleanup kit. The Dra III fragment was ligated to Van91 I cut pR1 to generate pR1-dHFR (noltr).

The 594 bp long MLV long terminal repeat, which contains a polyadenylation signal was amplified by PCR from pLNXH (TaKaRa Clontech) using the primers 5′-AAAAAATTAATTAAAATGAAAGACCCCACCTGTAGGTTTGG-3′ (SEQ ID NO:23) and 5′-AAAAAACACCGGTGAAAGTTTAAACAAACCTGCAGGAATGAAAGACCC CCGCTGACGGGTAG-3′ (SEQ ID NO:24). The PCR conditions that were used included 95° C. for 30 seconds, 56° C. for 30 seconds, and 72° C. for 45 seconds for 15 cycles using Pfu polymerase. The blunt-ended PCR product was then cloned into pCR-Blunt II-TOPO to create pCR-pLTR. The MLV LTR was cut out of pCR-pLTR using EcoRI, blunted-ended with Klenow, and gel purified. pR1-dHFR(noltr) was digested with PmeI and treated with CIP. The MLV LTR fragment containing the MLV poly A signal was ligated to the Pme I-digested vector to create pR1-DHFR. The orientations and correct sequences of the inserts wer confirmed by restriction enzyme digestions and DNA sequencing.

Construction of Donor-DHFR Expression Vector

The donor-DHFR expression vector (pD1-DHFR) can be constructed by cloning a mouse DHFR expression cassette consisting of the SV40 promoter, a mouse DHFR coding region, the 3′ UTR of the mouse DHFR cDNA, and the Moloney murine leukemia virus (MLV) polyadenylation signal into the donor expression vector pD1-DTX-1. This 1626 base pair expression cassette is amplified by PCR using Pfu polymerase from the target-DHFR vector pR1-DHFR using primers DHFR-1 (5′-TTTTTTGAAGACGAAAGGCTGTGGAATGTGTGTCAGTTAGGGTGTGGA-3′) (SEQ ID NO:25) and LTR-2 (5′-AAAAAACCTGCAGGAATGAAAGACCCCCGCTGACGGGTAG-3′) (SEQ ID NO:26), and cloned as a blunt-ended fragment into the BstZ17 I site of pD1-DTX-1 in the orientation shown in FIG. 16.

Construction of IRES-Donor Vector

The IRES-donor vector (pD1-IRES, FIG. 17) can be constructed by cloning two copies of the same IRES (also known as translational enhancer elements (TEEs)) into either the unique BamHI or Asc I sites of pD1-DTX-1. Several IRES can be chosen such as the naturally occurring Gtx IRES from the mouse Gtx homeodomain gene (Chappell, et al., 2000), the naturally occurring IRES in the mouse Rbm3 mRNA (Chappell, et al., 2003), or synthetic IRES such as ICS1-23b or ICS2-17.2 that were selected in a FACS-based enrichment scheme (Owens, et al., 2001). Multimeric versions of some IRES often enhance translation several fold better than monomeric versions. Sequences of IRES, even multimers, are short and are easily inserted into pD1-like vectors by constructing synthetic oligonucleotides that encode them.

A multimeric ICS1-23b IRES is assembled by annealing 2 synthetic oligonucleotides. One pair, consisting of the sequences 5′-GATCCAGCGGAAACGAGCGAAAAAAAAACAGCGGAAACGAGCGAAAA AAAAACAGCGGAAACGAGCGAAAAAAAAACAGCGGAAACGAGCGAAA AAAAAACAGCGGAAACGAGCGGACTCACAACCCCAGAAACAGACATG-3′ (SEQ ID NO:27) and 5′-GATCCATGTCTGTTTCTGGGGTTGTGAGTCCGCTCGTTTCCGCTGTTTTTT TTTCGCTCGTTTCCGCTGTTTTTTTTTCGCTCGTTTCCGCTGTTTTTTTTTC GCTCGTTTCCGCTGTTTTTTTTTCGCTCGTTTCCGCTG-3′ (SEQ ID NO:28), which have ends complementary to a BamH I restriction site and another pair, consisting of the sequences 5′-CGCGCCAGCGGAAACGAGCGAAAAAAAAACAGCGGAAACGAGCGAAA AAAAAACAGCGGAAACGAGCGAAAAAAAAACAGCGGAAACGAGCGAA AAAAAAACAGCGGAAACGAGCGGACTCACAACCCCAGAAACAGACAT GG-3′ (SEQ ID NO:29) and 5′-CGCGCCATGTCTGTTTCTGGGGTTGTGAGTCCGCTCGTTTCCGCTGTTTT TTTTTCGCTCGTTTCCGCTGTTTTTTTTTCGCTCGTTTCCGCTGTTTTTTTT TCGCTCGTTTCCGCTGTTTTTTTTTCGCTCGTTTCCGCTGG-3′ (SEQ ID NO:30), that have ends complementary to an Asc I restriction site. These sequences contain 5 copies of the 15 base long ICS1-23b IRES. Each is separated by a four copies of a 9 base long poly A spacer. Finally, the 3′ end contains a 25 base sequence that immediately precedes the mouse β-globin coding region (e.g., GenBank Accession Number J00413). These annealed oligonucleotides are cloned into the BamH I and Asc I sites of pD1-DTX-1 to create the IRES-donor vector pD1-IRES. Clones are sequenced to identify those with the correct orientation and sequence.

Construction of Regulatable Target Vector

When some proteins are expressed at levels necessary to render them commercially useful they can be toxic and lead to slow cell growth or even cell death. Therefore, it can be useful to repress their expression until it is necessary to produce large quantities. Several methods for regulating genes are available. In some embodiments, it is desirable to introduce the system which regulates genes into cells first before the protein expression cassette is introduced into cells. In this manner the gene regulatory system is established and will repress gene expression before an expression vector is introduced. Therefore, it may be desirable to have a gene regulatory system on the target vector pR1 and not the donor vector.

The RheoSwitch system (New England Biolabs) provides gene regulation over a wide expression range. Gene regulation by the RheoSwitch system is mediated by two proteins. The RheoReceptor consists of the yeast GAL4 protein fused to the ligand binding domain of an insect estrogen nuclear receptor. The RheoReceptor binds to upstream activating sequences (UAS) derived from the yeast GAL4 gene that is placed upstream of a TATA-box. The RheoActivator consists of a hybrid insect/mammalian RXR ligand binding receptor fused to the herpes simplex virus VP16 transcriptional activation domain. Ecdysone analogs can dimerize the RheoReceptor and the RheoActivator and when this occurs genes that are properly linked to GAL4 UAS DNA binding elements will be activated. Furthermore in the absence of the dimerizer the RheoReceptor binds to the UAS sequences and mediates repression of gene expression. The net result is that basal levels of expression using this system are very low and the levels of induction that can be achieved are high.

Gene cassettes encoding the two protein components of the RheoSwitch system (RheoReceptor and RheoActivator) can be amplified by PCR from pNEBR-R1 (New England Biolabs). They are cloned in an orientation, as shown in FIG. 18, such that the coding regions for the RheoReceptor and RheoActivator are in an orientation that is the same as that of the puromycin coding region. This configuration is different from the configuration in pNEBR-R1 (where they are in opposite orientations) and this is why the RheoReceptor and RheoActivator gene cassettes are cloned into pR1 separately.

More specifically, PCR primers consisting of the sequences 5′-AAAAAAACCCTGCAGGGGCCTCCGCGCCGGGTTTTGGCGCCT-3′ (SEQ ID NO:31) and 5′-AAAAAAAACACCGGTGCTTATCGGATTTTACCACATTTG-3′ (SEQ ID NO:32) are used to amplify the RheoActivator gene expression cassette (which consists of a ubiquitin C (UbC) promoter, RheoActivator coding region, and SV40 late region polyadenylation signal sequence). The 2481 base pair long product is digested with Sbf I and SgrA I and cloned into the unique Sbf I/SgrA I sites of pR1-PL1 to create pR1-RA.

PCR primers consisting of the sequences 5′-AAAAAAAACACCGGTGCCGATATCGGGTGCCACGCCGTCCCG-3′ (SEQ ID NO:33) and 5′-AAAAAAAAGCCCGGGCGGCGGCCCGCCAGAAATCC-3′ (SEQ ID NO:34) are used to amplify the RheoReceptor gene expression cassette (which consists of a ubiquitin B (UbB) promoter, RheoReceptor coding region, and TK polyadenylation signal sequence). The 3680 base pair long product is digested with SgrA I and Srf I and cloned into the unique SgrA I/Srf I sites of pR1-RA to create pRlreg.

Construction of Regulatable Target-DHFR Vector

In order to construct a target vector that can regulate genes in the donor vector and be subjected to gene amplification, a regulating target-DHFR vector (FIG. 19) is constructed. The gene regulating cassette from pRlreg, consisting of the RheoActivator and RheoReceptor genes, is amplified by PCR from pRlreg using primers 5′-AAAAAAACCCTGCAGGGGCCTCCGCGCCGGGTTTTGGCGCCT-3′ (SEQ ID NO:35) and 5′-AAAAAAAAGCCCGGGCGGCGGCCCGCCAGAAATCC-3′ (SEQ ID NO:36), digested with Sbf I and Sfr I and cloned into the Sbf I and Sfr I sites of pR1-DHFR to construct the regulating target-DHFR vector pR1reg-DHFR

Construction of Regulatable Donor Expression Vector Backbone

The regulatable donor expression vector backbone (FIG. 20) has the DNA sequences recognized by the protein component (e.g., RheoReceptor) of the gene regulatory system encoded by pRlreg cloned upstream of coding regions for proteins of interest. In the case of the RheoSwitch system the DNA elements that the RheoReceptor binds to are GAL4 upstream activation sequences (UAS). A 722 base pair long DNA sequence encoding, in order, restriction sites (the 3′ half of BstZ17 I, EcoR I), the SV40 polyadenylation signal region (to prevent cryptic transcription into the regulatory region), five GAL4 UAS elements, and a TATA box can be amplified by PCR from pNEBR-X1Hygro (New England Biolabs) using primers 5′-TACGAATTCATCAGCCATATCACATTTGTAGAG-3′ (SEQ ID NO:37) and 5′-TTATATACCCTCTAGAGTCTCCGCTCGGA-3′ (SEQ ID NO:38).

Two 173 or 178 base pair long DNA sequences encoding two versions of the CMV early promoter 5′ untranslated region (5′ UTR) with different restriction enzyme sites on the 3′ ends are generated by annealing two sets of overlapping oligonucleotides and filling in their 3′ ends using Klenow DNA polymerase. The 173 base long version is generated by annealing 5′-CCGAGCGGAGACTCTAGAGGGTATATAAGCAGAGCTCGTTTAGTGAAC CGTCAGATCGCCTGGAGACGCCATCCACGCTGTTTTGACCTCCATAGAA GAC-3′ (SEQ ID NO:39) and 5′-AAAAAAGGATCCGAGCTCGGTACCAAGCTTCCAATGCACCGTTCCCGGC CGCGGAGGCTGGATCGGTCCCGGTGTCTTCTATGGAGGTCAAAA-3′ (SEQ ID NO:40) and filling in with Klenow polymerase. The 178 base long version is generated by annealing 5′-CCGAGCGGAGACTCTAGAGGGTATATAAGCAGAGCTCGTTTAGTGAAC CGTCAGATCGCCTGGAGACGCCATCCACGCTGTTTTGACCTCCATAGAA GAC-3′ (SEQ ID NO:41) and 5′-AAAAAAGGCGCGCCGAATTCACCGGTACCAAGCTTCCAATGCACCGTTC CCGGCCGCGGAGGCTGGATCGGTCCCGGTGTCTTCTATGGAGGTCAAAA 3′ (SEQ ID NO:42) and filling in with Klenow polymerase. Then they are mixed separately with the 722 base pair PCR product (containing the SV40 poly A signal, five GAL4 UAS, and a TATA box), and PCR amplified with two sets of PCR primers: either 5′-TACGAATTCATCAGCCATATCACATTTGTAGAG-3′ (SEQ ID NO:43) and 5′-AAAAAAGGATCCGAGCTCGGTACCAAGCTTCCAATGCACCGTTCCCGGC CGCGGAGGCTGGATCGGTCCCGGTGTCTTCTATGGAGGTCAAAA-3′ (SEQ ID NO:44) or 5′-TACGAATTCATCAGCCATATCACATTTGTAGAG-3′ (SEQ ID NO:45) and 5′-AAAAAAGGCGCGCCGAATTCACCGGTACCAAGCTTCCAATGCACCGTTC CCGGCCGCGGAGGCTGGATCGGTCCCGGTGTCTTCTATGGAGGTCAAAA-3′ (SEQ ID NO:46).

In this manner two cassettes containing a SV40 polyadenylation signal region (to prevent cryptic transcription into the regulatory region), five GAL4 UAS elements, a TATA box, and a 5′ UTR from the CMV early promoter are assembled. One is digested with EcoR I and BamH I and cloned into the Mfe I/BamH I site of pHPC-4 to create pHPC-4reg. The other is digested with Asc I and cloned into the BstZ17 I/Asc I site of pHPC-4reg to create pD1reg. Both of these cloning steps remove the two constitutive CMV promoters in pHPC-4 which could interfere with regulated expression. As described above, various genes of interest can be inserted into the polylinker regions of pD1reg such that they can be integrated into a target vector and their expression can be regulated.

There are two features about the construction of pD1reg that may be important for maintaining the high levels of gene expression possible using versions of the donor vector that do not contain components of a gene regulatory system (e.g., pD1, pD1-DHFR, pD1-IRES). First the TATA box from the gene regulatory system was precisely fused to the TATA boxes from the CMV promoters of pD1. Second, the 5′ UTRs of the CMV promoters were reconstituted. The net result is that the sequences between the TATA box and the translation start codon (i.e., the transcription start site and the 5′ UTR) of pD1reg are the same as they are in pD1. However the sequences before the TATA boxes in pD1reg consist of those DNA sequences required to obtain gene regulation mediated by the protein components of the gene regulatory system that are encoded by pR1reg.

Construction of a Selectable Donor Expression Vector

The selectable donor expression vector (FIG. 21) is similar to the Donor Expression Vector except that it also includes a complete drug resistance gene, which is different from both the promoterless first selectable marker gene and the second functional selectable marker gene on the target vector. By way of example the construction of a selectable donor expression vector with a complete G418 resistance gene (pD1-DTX1-G418, FIG. 21) is described. The sequence of the pD1-DTX1-G418 vector is provided in FIGS. 36A-36D.

The selectable donor expression vector pD1-DTX1-G418 was constructed by amplifying a complete, functional G418 drug resistance cassette from pcDNA3002neo (Crucell) using the polymerase chain reaction and the primers 5′-GAGAGAGGATCCACGCGTCTGTGGAATGTGTGTCAGTTAGGG-3′ (SEQ ID NO:47) and 5′-GAGAGAGAATTCTCTAGACAGACATGATAAGATACATTGATGAGTTTG-3′ (SEQ ID NO:48). The resulting PCR product contains an SV40 promoter, the G418 resistance gene, and the SV40 poly adenylation signal. The PCR product was digested with the restriction enzymes BamH I and EcoR I and ligated into the donor expression vector pD1-DTX-1, which had been digested with Bgl II and Mfe I. The ligation was digested with Bgl II and Mfe I (which are destroyed by ligation of the insert) to reduce ligation of vector backbone alone and transformed into XL-10 Gold ultracompetent E. coli cells (Stratagene). Clones with inserts in the desired oritentation were identified by PCR and restriction enzyme digestion. The correct DNA sequence of the entire G418 resistance gene was confirmed by sequencing.

Construction of a Reporter Donor Expression Vector

The reporter donor expression vector (FIG. 30) is similar to the Donor Expression Vector except that it also includes a reporter gene, which can be detected in individual cells either by, for example, fluorescence microscopy or a fluorescence activated cell sorter. In general, the expression level of the reporter gene on a reporter donor expression vector will correlate to the expression level of proteins of interest on the same reporter donor expression vector. Therefore, after transfection of target vector clones with a reporter donor expression vector, target vector clones can be optionally identified that result in high level expression of a protein of interest by identifying clones that express the reporter gene at high levels. By using a high throughput instrument such as a fluorescence activated cell sorter a much larger number of target vector clones (i.e., integration sites) can be screened for expression than can be screened by manual clone picking methods.

In such an optional scheme a large number of pools of target vector clones will be generated. For example, cells will be transfected with a target vector and a first integrase expression vector. Stable colonies will be selected (e.g, by resistance to hygromycin). For example, as many as 100 plates with 100 colonies per plate (i.e., 10,000 target vector clones) can be generated. Each pool of target vector clones is then transfected separately with a reporter donor expression vector and a second integrase expression vector. Stable integration of reporter donor expression vectors into target vectors is selected (e.g, by resistance to puromycin). Each individual pool of reporter donor vector clones is sorted using a fluorescence activated cell sorter and single cells from each pool with the highest reporter gene expression are collected. High level expression of the protein of interest is then confirmed. The integration site of the target vector in cells with the highest reporter gene expression is then determined using plasmid rescue or PCR techniques. Target vector-specific PCR primers are designed to be specific for the target vector integration sites. Then, the pools of target vector clones that provide the highest levels of expression are single cell cloned and the target vector-specific PCR primers are used to identify which individual target vector clones that give rise to the highest levels of expression after transfection with a reporter donor expression vector and a second integrase expression vector. By isolating a small number of target vector clones that result in the very highest levels of protein expression, other donor expression vectors can be transfected into the identified clones to express a variety of other proteins, instead of doing the large scale expression screening each time.

In addition to the optional use described above for high throughput screening of integration sites, a reporter donor expression vector provides a simple, quick method for monitoring the time course, frequency, and stability of reporter donor vector integration in real time by examination of transfected cells using a fluorescence microscope. By way of example the construction of a reporter donor expression vector with a green fluorescent protein gene (pD3-DTX1, FIG. 30) is described.

The reporter donor expression vector pD3-DTX1 was constructed by first amplifying a Rous Sarcoma Virus promoter (pRSV) from the plasmid pLXRN (Clontech) using the polymerase chain reaction and the primers 5′-TTTTCACTGCATTCGACAATTGTCATCCCCTCAGGATATAGTAGTTTC-3′ (SEQ ID NO:49) and 5′-GACCAGCACGTTGCCCAGGAGTTGGAGGTGCACACCAATGTGGTG-3′ (SEQ ID NO:50). A DNA containing the humanized Renilla reniforms green fluorescent protein (hrGFP) coding region and a human growth hormone (hGH) gene polyadenylation signal was amplified by PCR from pAAV hrGFP (Stratagene) using the primers 5′-CACCACATTGGTGTGCACCTCCAACTCCTGGGCAACGTGCTGGTC-3′ (SEQ ID NO:51) and 5′-GAGAGAGCTAGCATTTAAATAAGGACAGGGAAGGGAGCAGTGG-3′ (SEQ ID NO:52). The 2 PCR products were mixed and amplified with the primers 5′-TTTTCACTGCATTCGACAATTGTCATCCCCTCAGGATATAGTAGTTTC-3′ (SEQ ID NO:53) and 5′-GAGAGAGCTAGCATTTAAATAAGGACAGGGAAGGGAGCAGTGG-3′ (SEQ ID NO:54) in order to fuse the Rous Sarcoma Virus promoter to the hrGFP coding region and the hGH gene polyadenylation signal. The resulting blunt-ended PCR product was ligated into the blunt Psi I site of the donor expression vector pD1-DTX1. Clones with inserts were identified by PCR using the primers 5′-TTTTCACTGCATTCGACAATTGTCATCCCCTCAGGATATAGTAGTTTC-3′ (SEQ ID NO:53) and 5′-GAGAGAGCTAGCATTTAAATAAGGACAGGGAAGGGAGCAGTGG-3′ (SEQ ID NO:54) and the orientation of the insert was determined by restriction enzyme digestion. The correct DNA sequence of the entire pRSV-hrGFP-hGH poly A insert was confirmed. The sequence of the pD3-DTX1 vector is provided in FIGS. 37A-37D.

### Testing of Vectors

The functions of the individual target vector, donor expression vector, and integrase expression vectors was tested. For example transfection of the target vector into either DG44 cells or PER.C6™ cells can confer hygromycin resistance. When either the R4 integrase expressing vector or the φC31 integrase expressing vector is transfected with the target vector about 5 times as many hygromycin resistant colonies resulted compared to transfection of the target vector alone showing that expression of either integrase can result in an increased number of stable clones. Transient transfection of the donor expression vector alone resulted in production of 300 ng/ml antibody in DG44 cells and 1 μg/ml in PER.C6™ (FIG. 31).

Another important function to demonstrate is the ability of the φC31 attP site in a target vector to recombine with the φC31 attB site in a donor expression vector. This is particularly true since the att sites in both the target vector and the donor vector were either mutated or truncated to meet the demands of the expression system described herein. DG44 cells (3e6) on 10 cm plates were transfected with 500 ng of a target vector (pR1) and 500 ng of a donor expression vector (pD1-DTX-1) in the presence or absence of 4000 ng of a φC31 integrase expressing vector (pCS-M3J) using Lipofectamine 2000 CD. Forty eight hours after transfection the cells were trypsinized and plasmid DNA was isolated using a QIAprep Spin Miniprep Kit (QIAGEN). The DNA was amplified with PCR primers 5′-TGCCCCGGGGCTTCACGTTTTCC-3′ (SEQ ID NO:55) (from φC31 att P) and 5′-GCCCGCCGTGACCGTCGAGAAC-3′(SEQ ID NO:56) (from φC31 att B), then with primers 5′-CAGGTCAGAAGCGGTTTTCGGGAG-3′ (SEQ ID NO:57) (from φC31 att P) and 5′-CCGCTGACGCTGCCCCGCGTATC-3′ (SEQ ID NO:58) (from φC31 att B), all of which were designed to specifically amplify the attR product that could result only from φC31 integrase-mediated recombination of a φC31 attP site in a target vector with a φC31 attB site in a donor expression vector. As a positive control 500 ng each of the plasmids pTA-attB and pTA-attP which contain longer, wild type φC31 att sites sequences were transfected in the presence or absence of 4000 ng of a φC31 integrase vector (pCS-M3J). pTA-attB and pTA-attP have 285 and 221 base pair long regions from the φC31 attB sites and φC31 attP sites, respectively. As a negative control untransfected cells were used. As can be seen in FIG. 22 pR1 and pD1-DTX-1 can recombine to generate an attR site only in the presence of φC31 integrase.

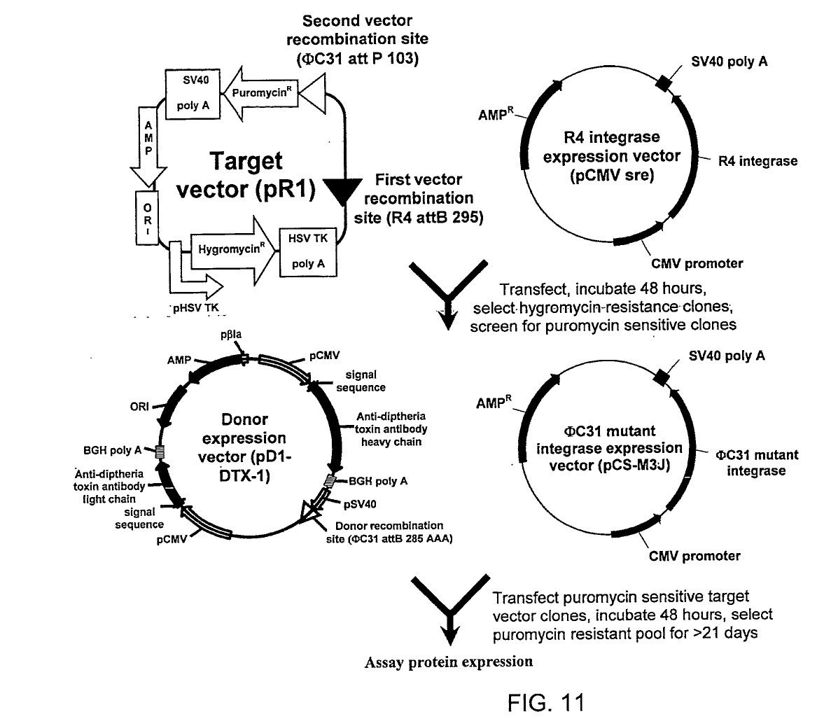

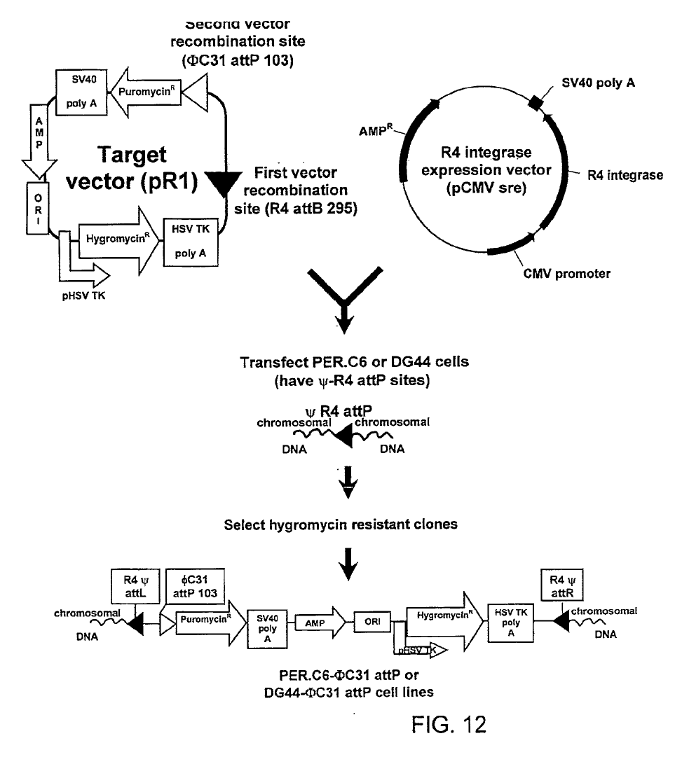

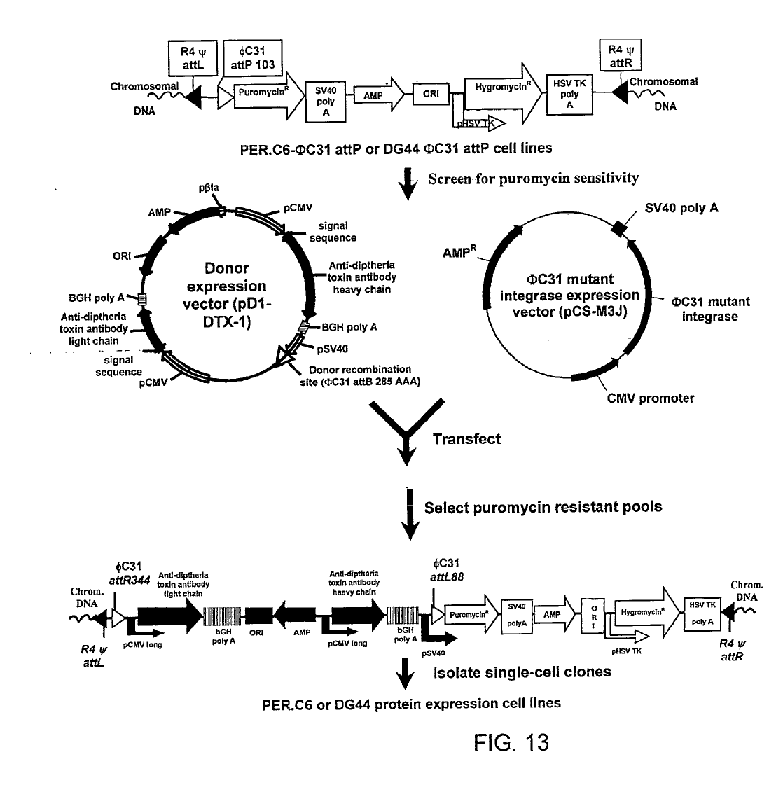

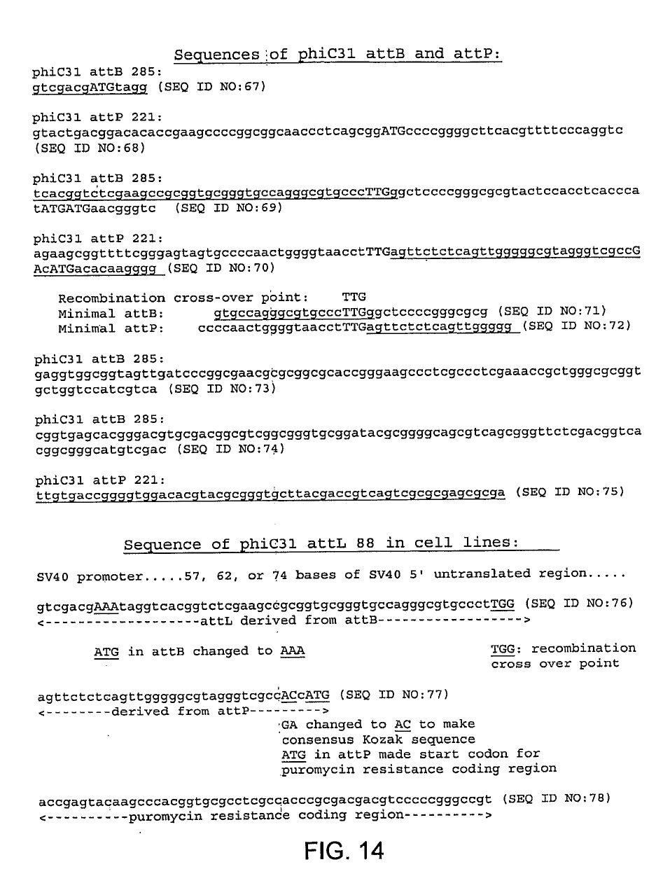

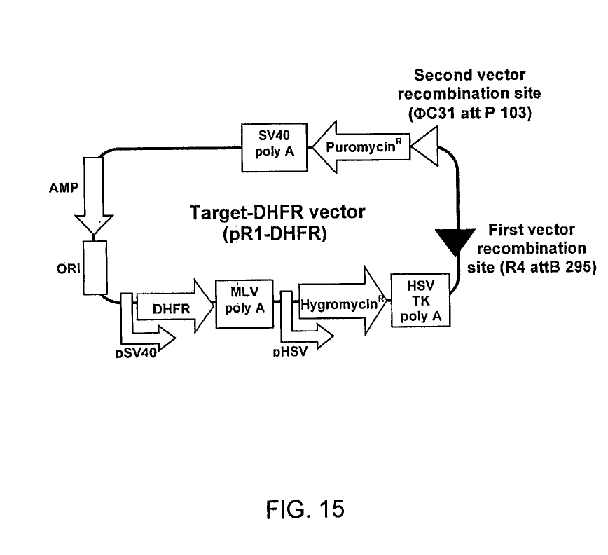

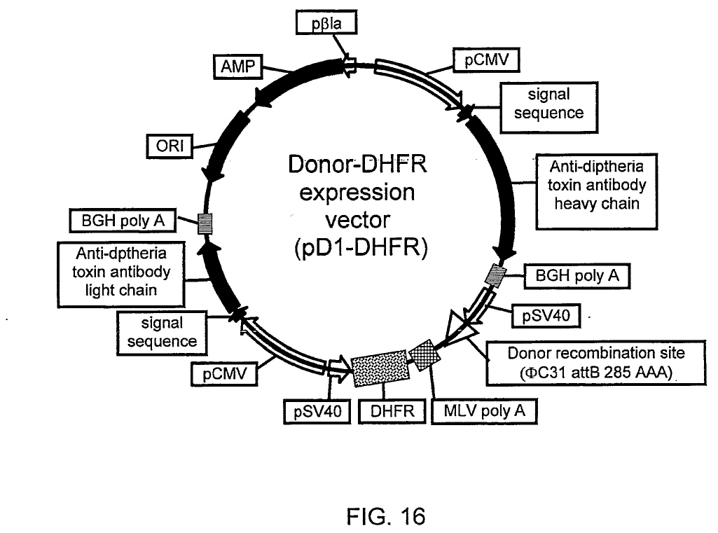

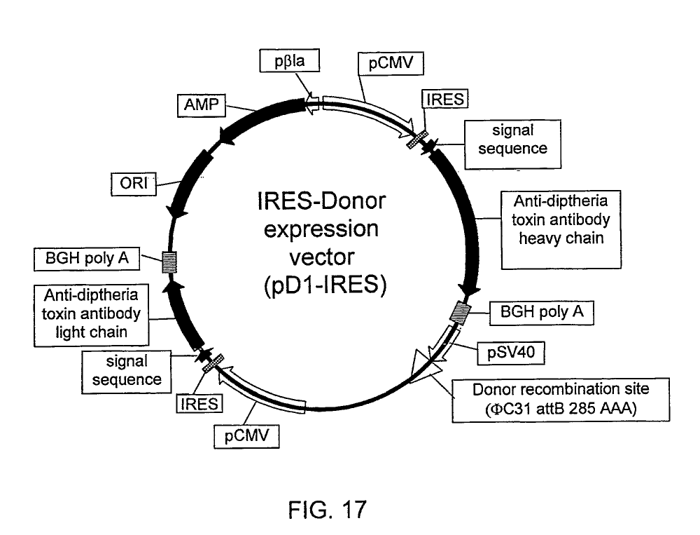

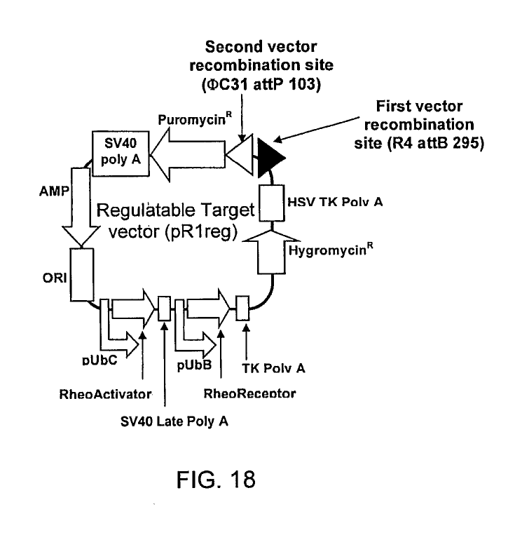

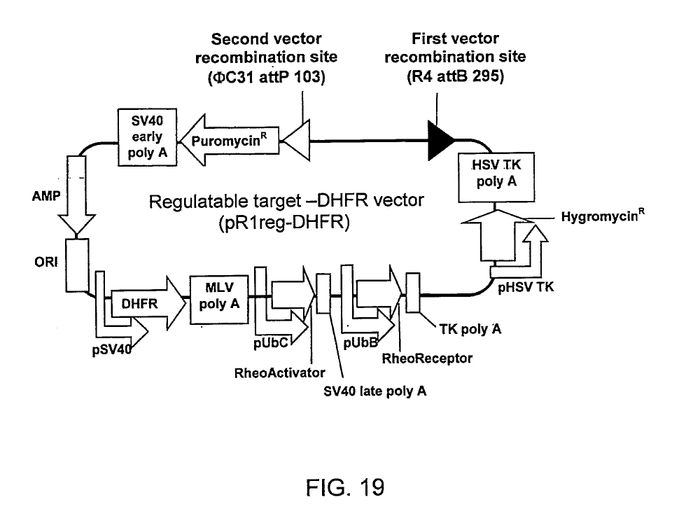

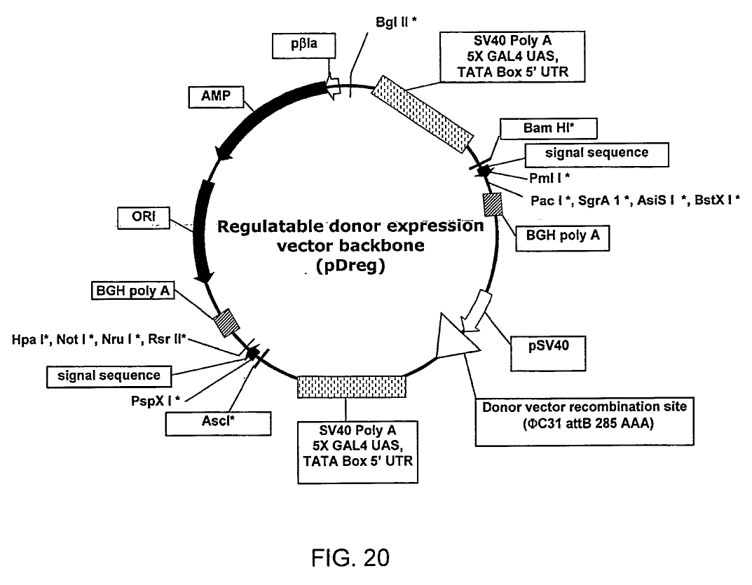

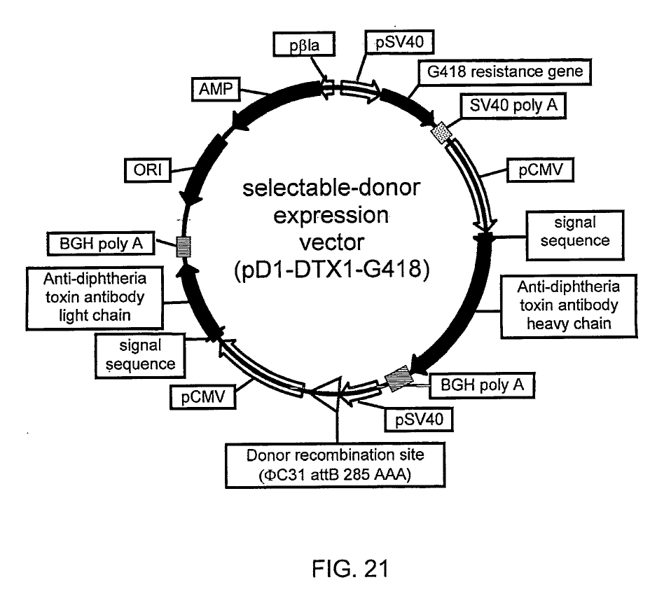

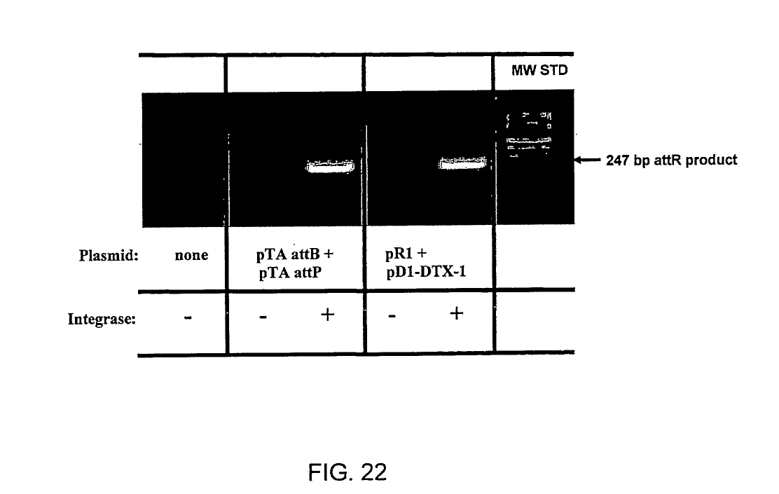

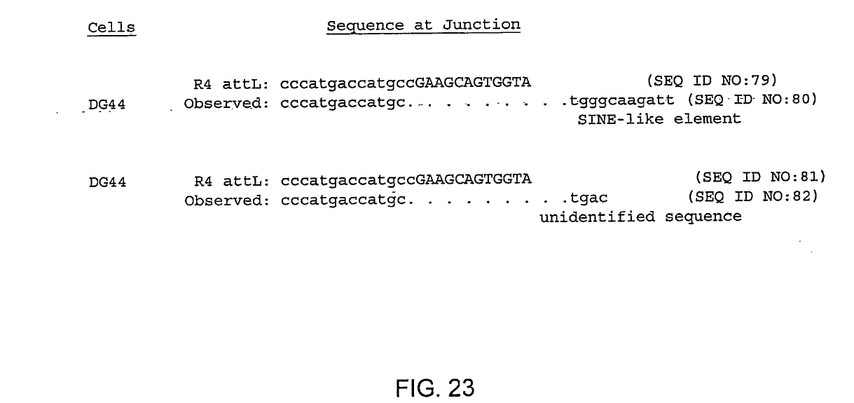

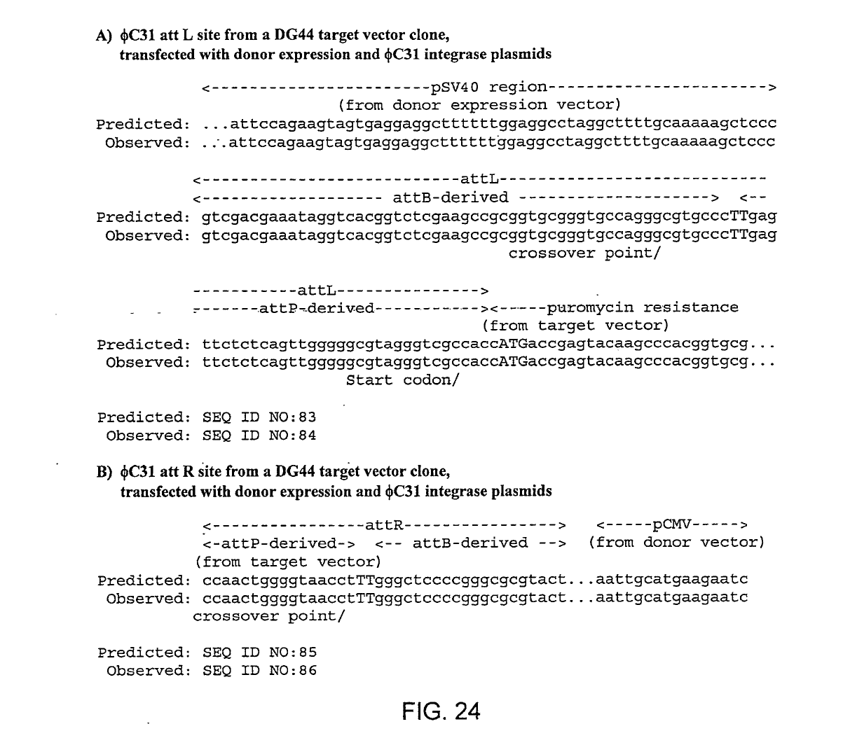

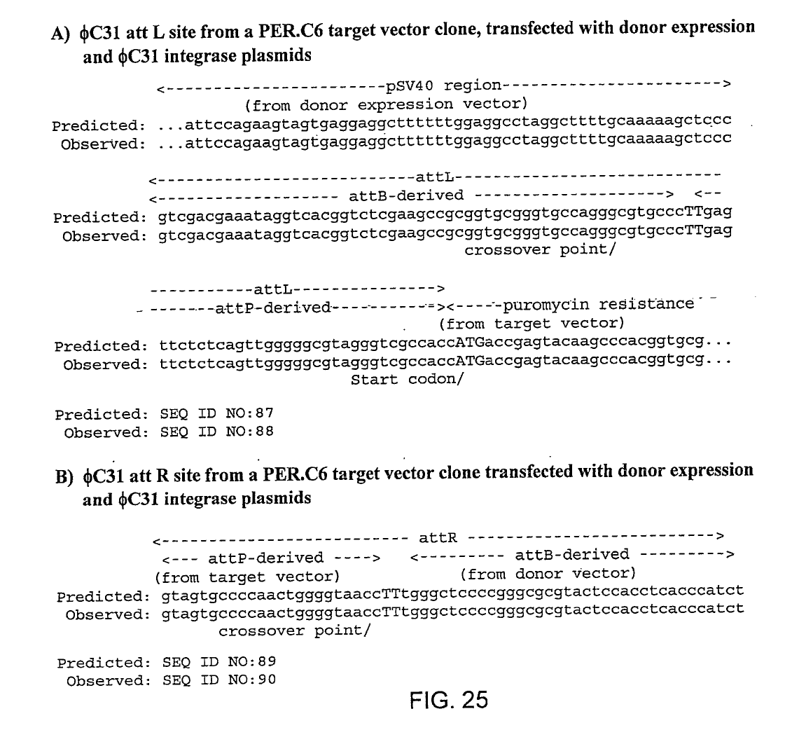

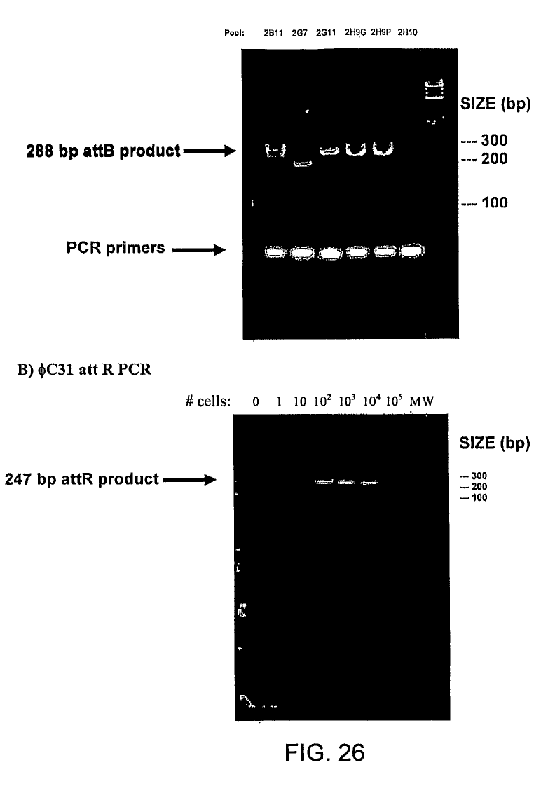

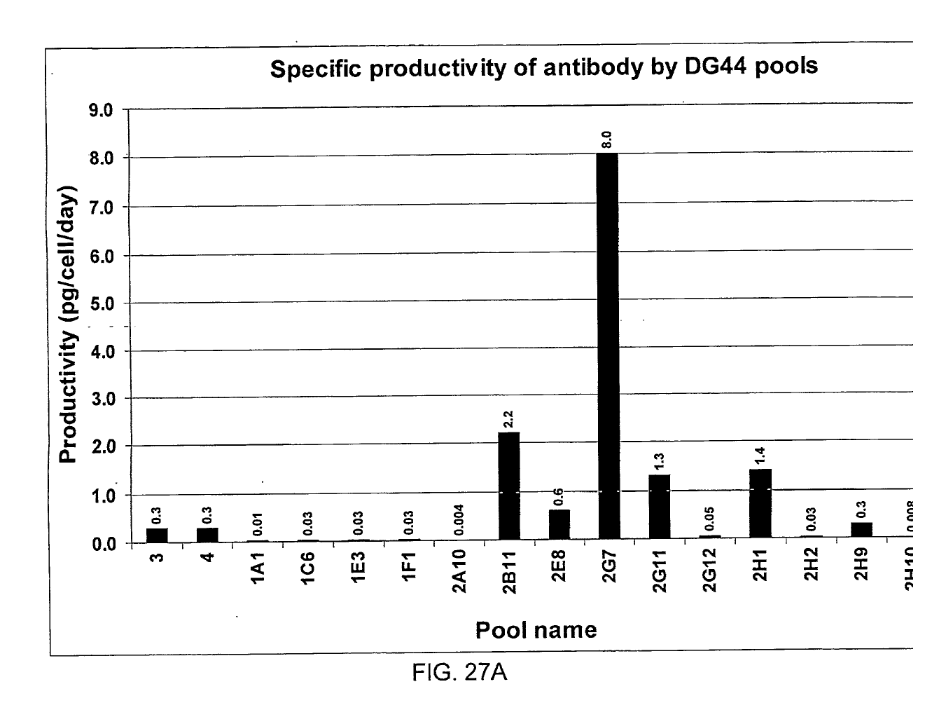

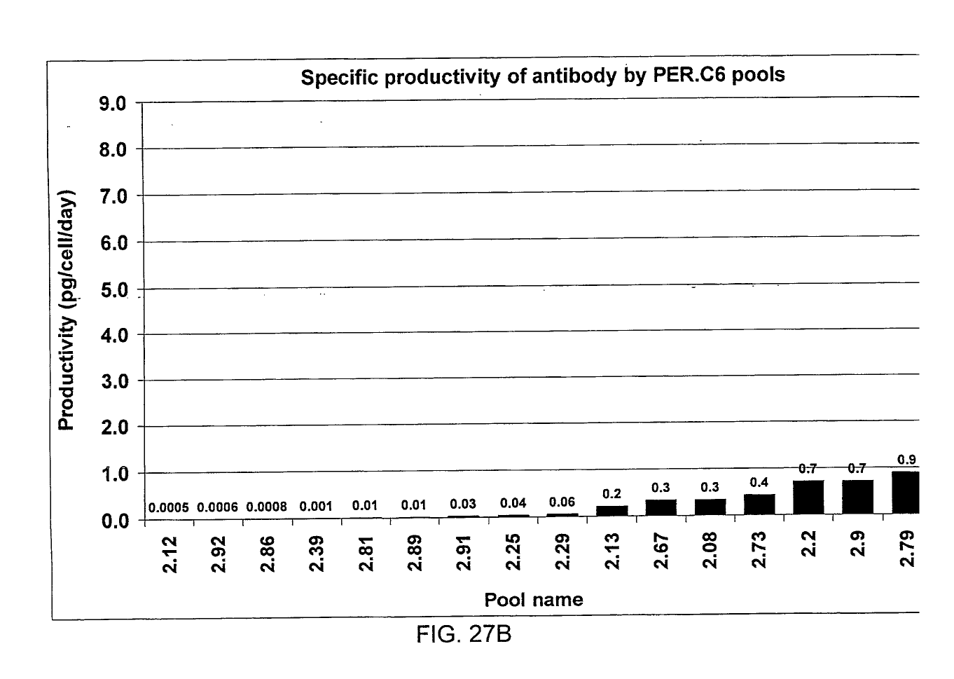

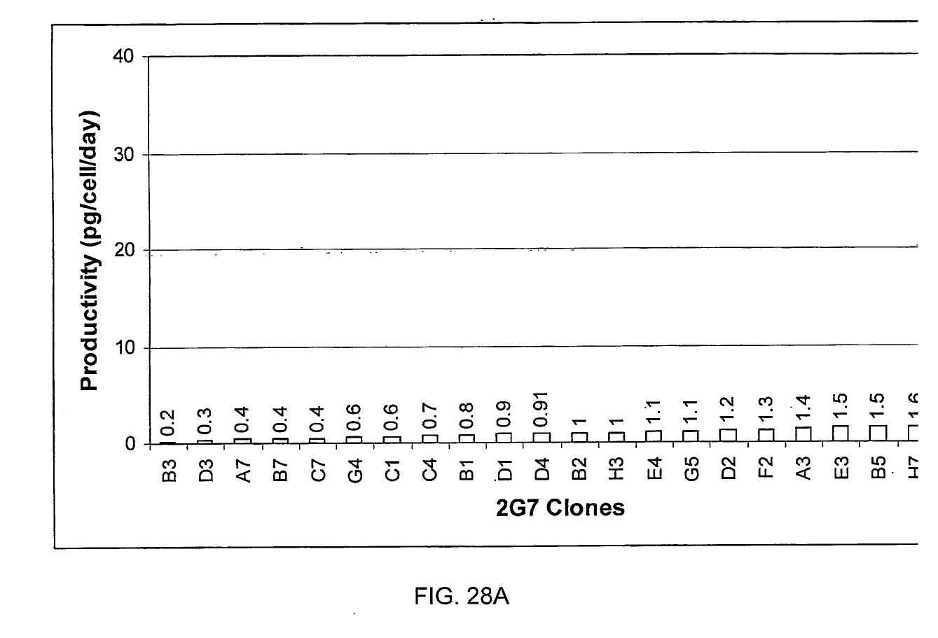

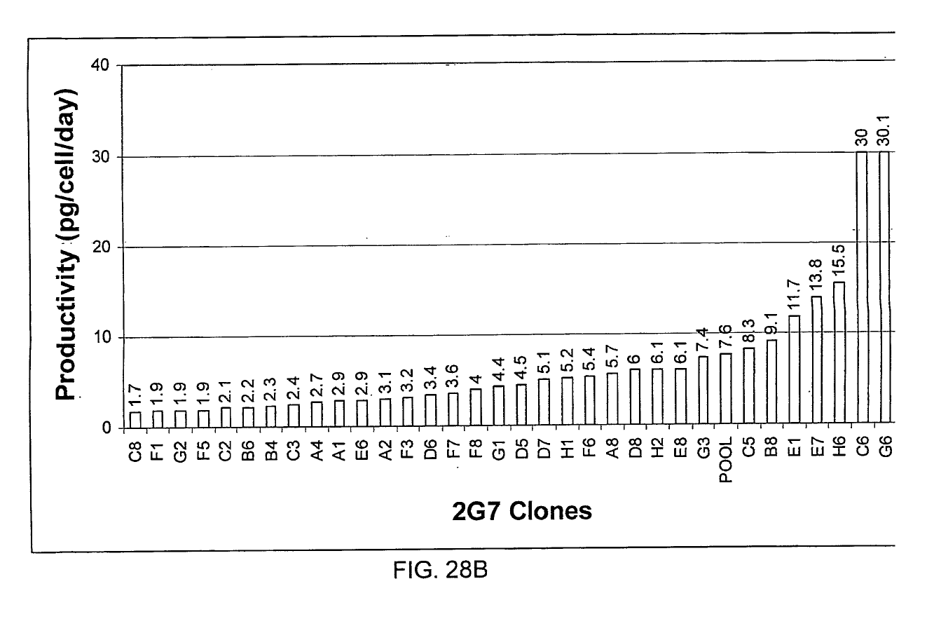

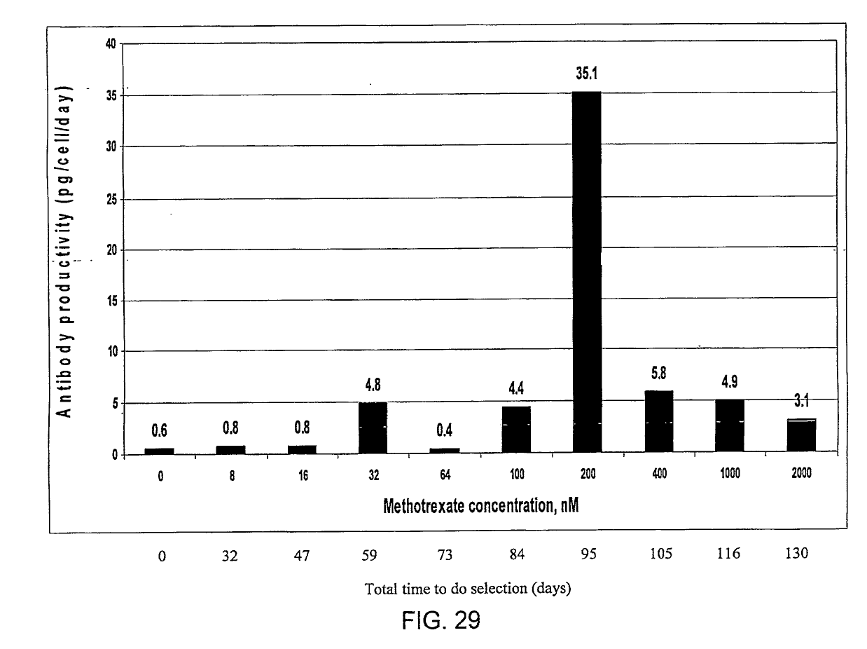

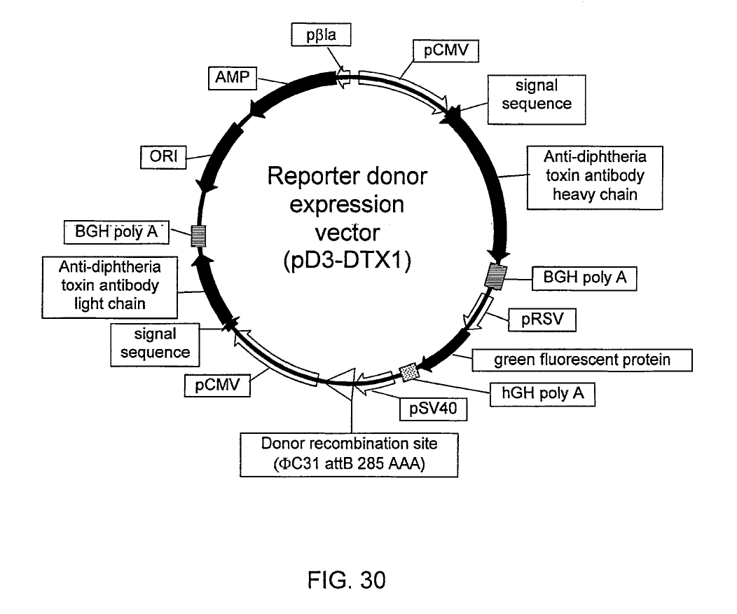

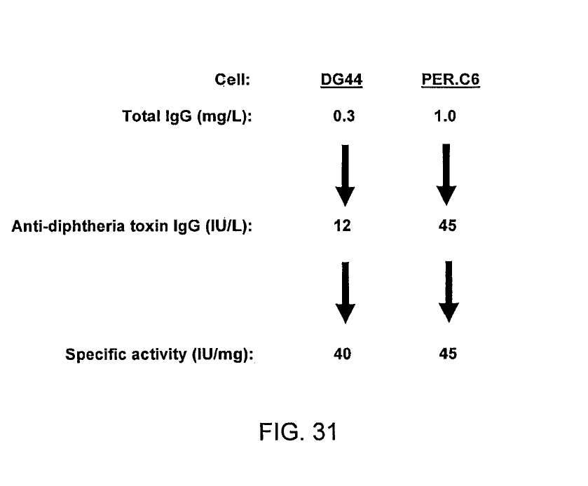

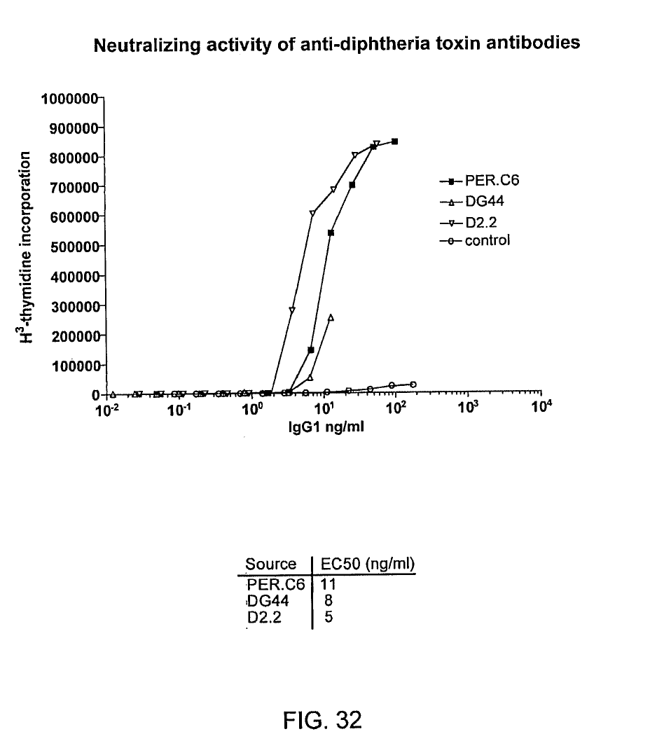

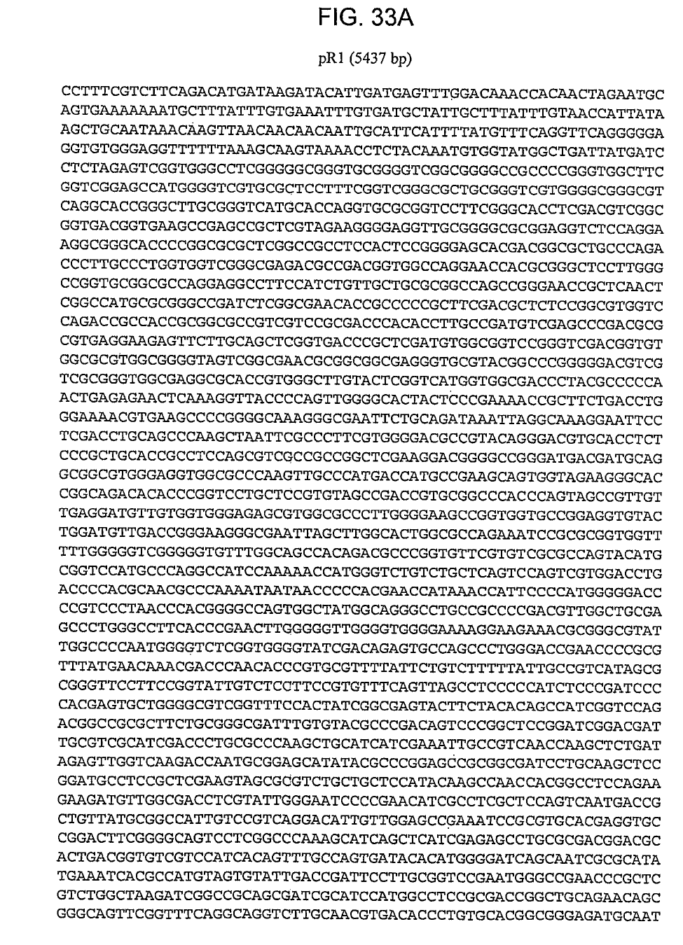

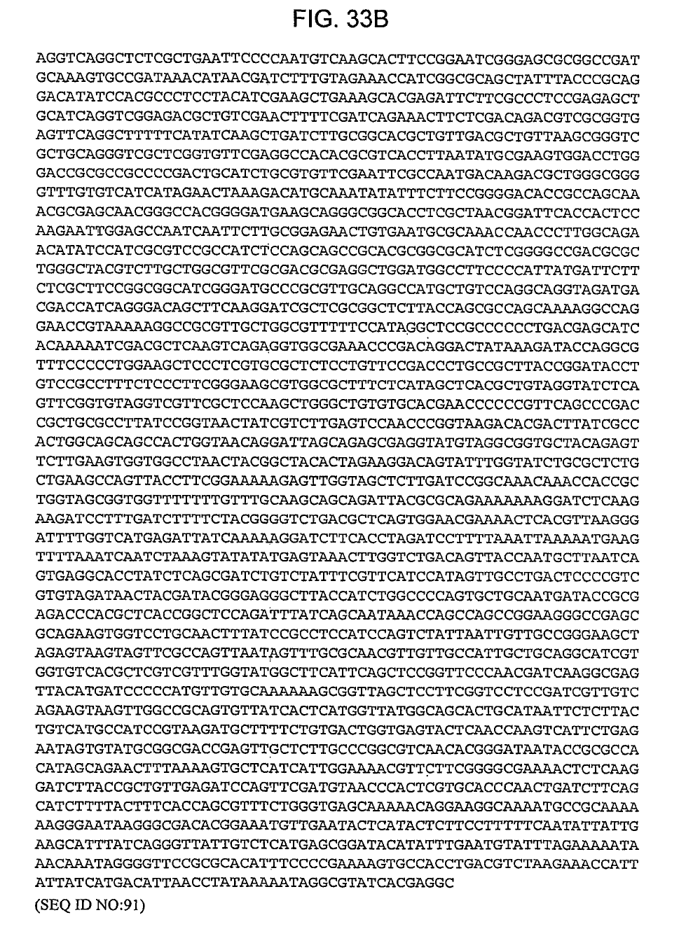

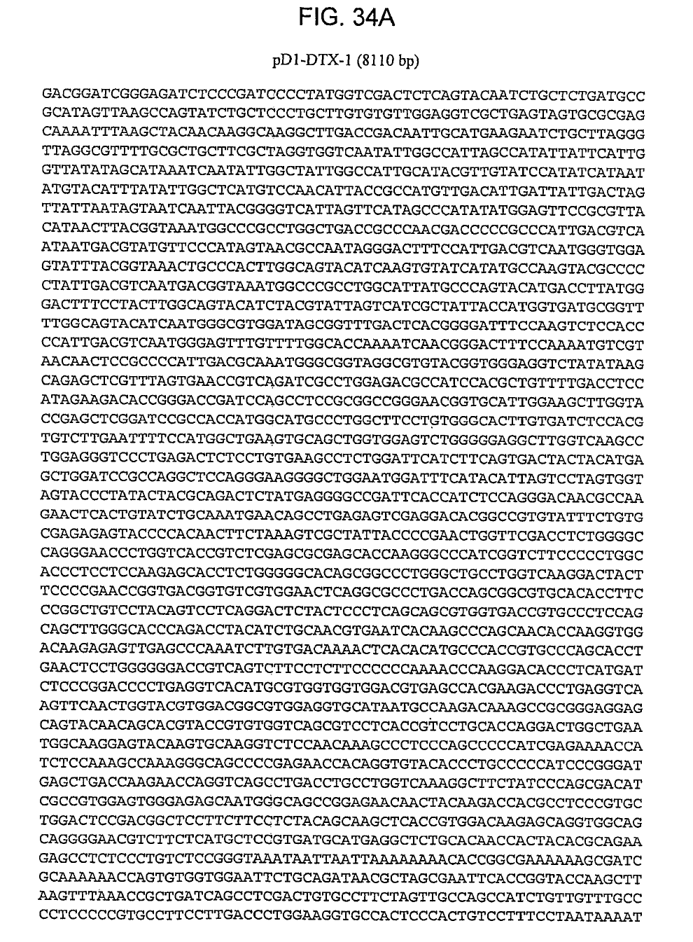

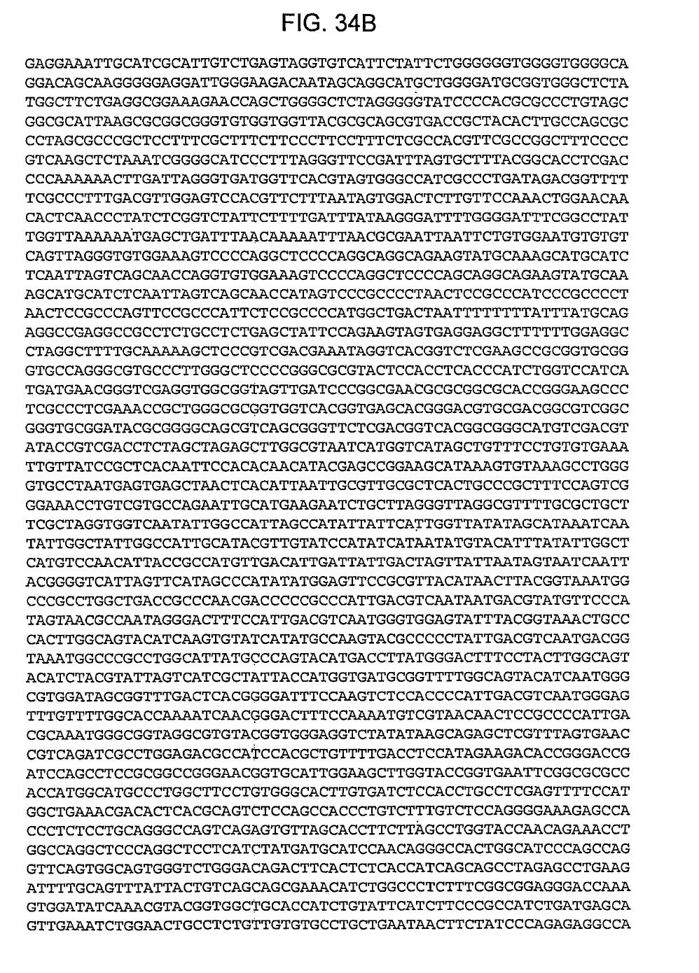

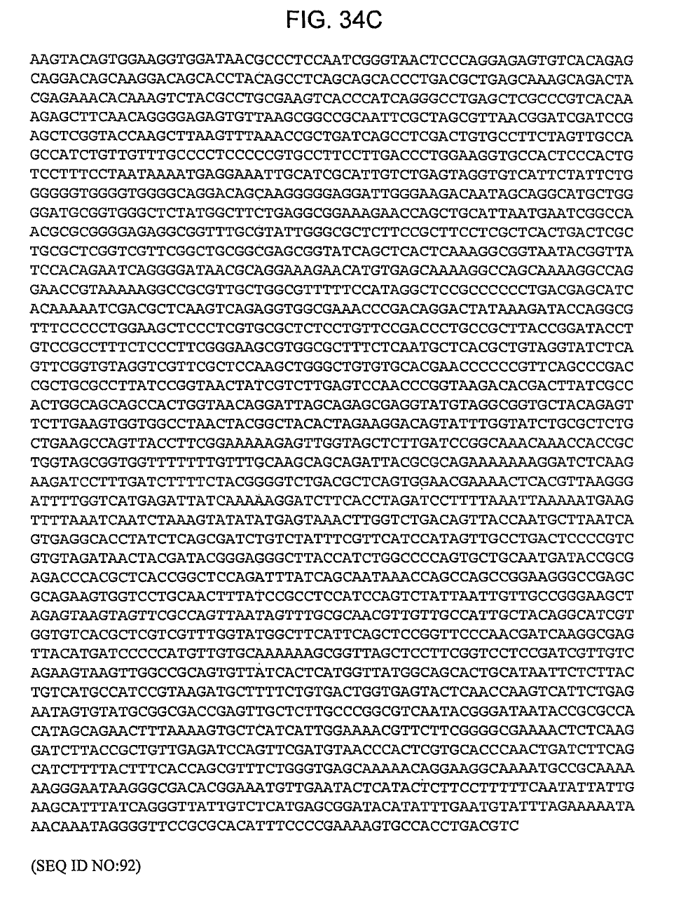

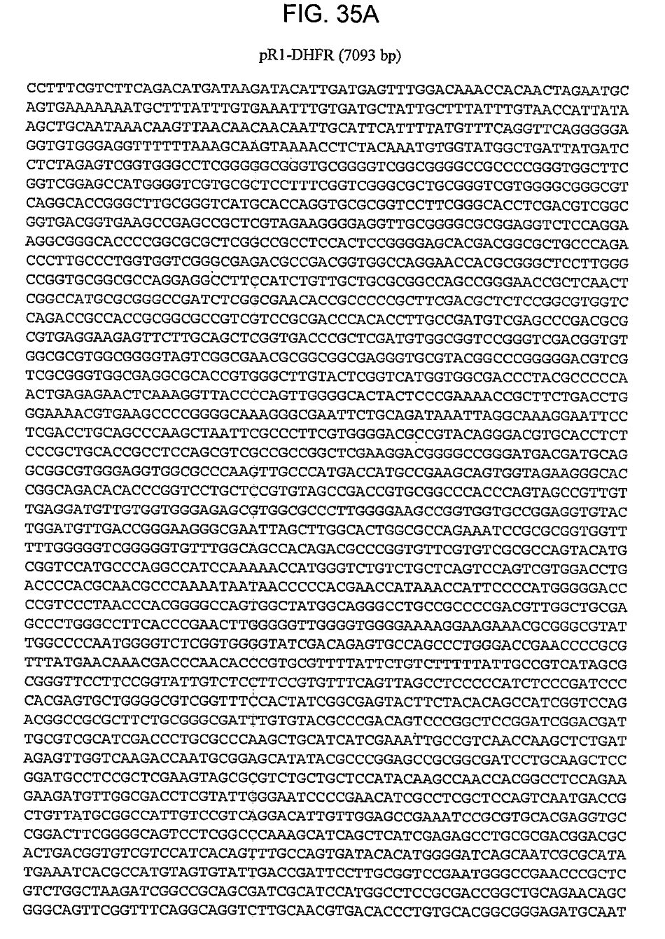

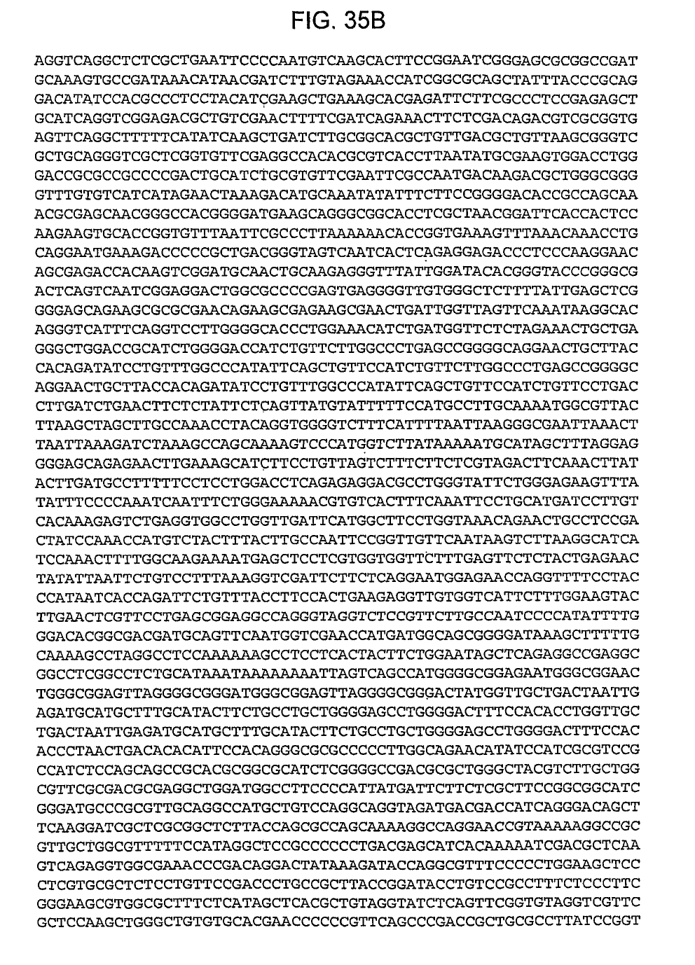

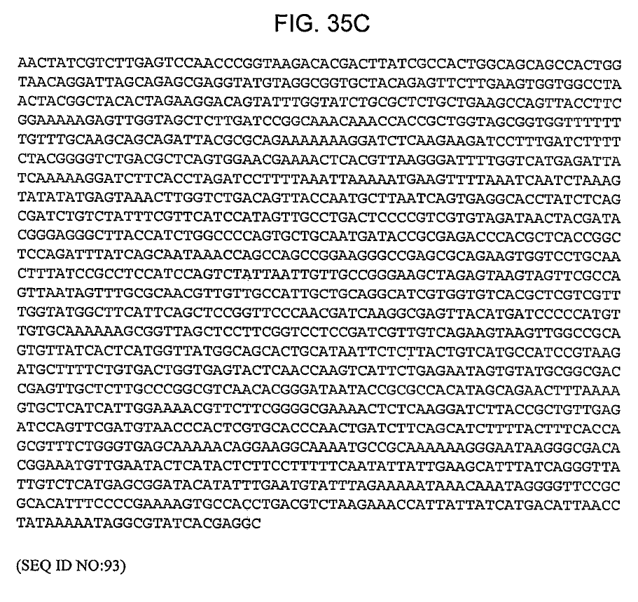

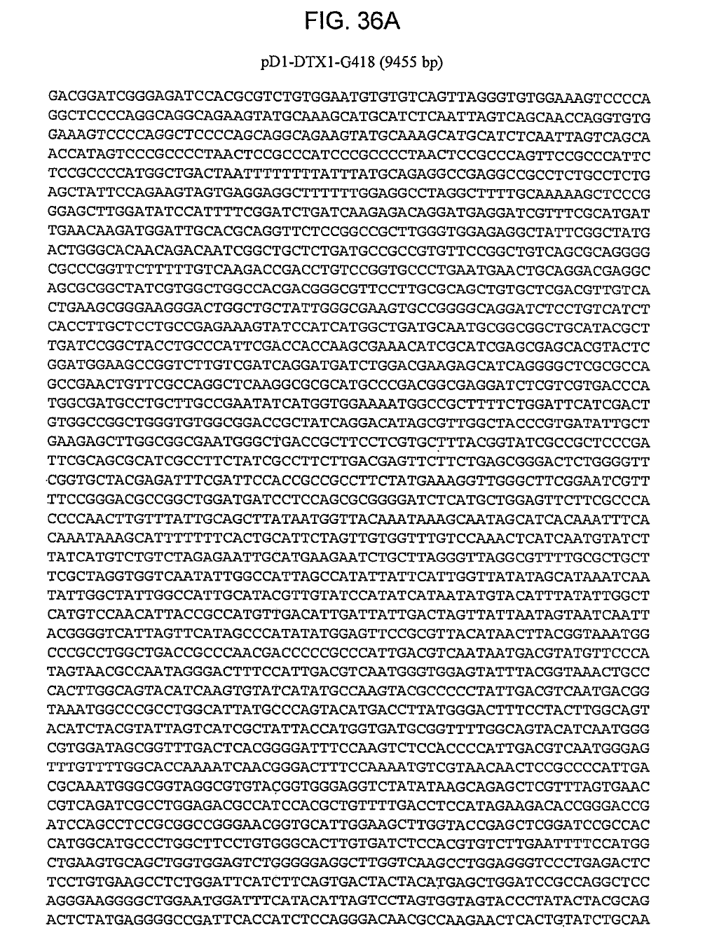

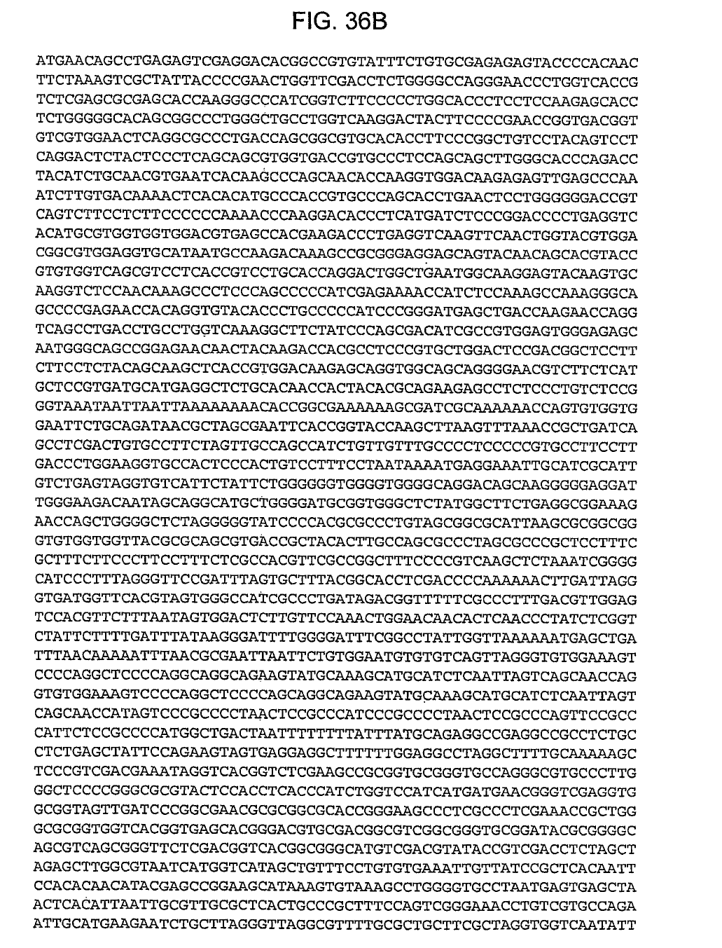

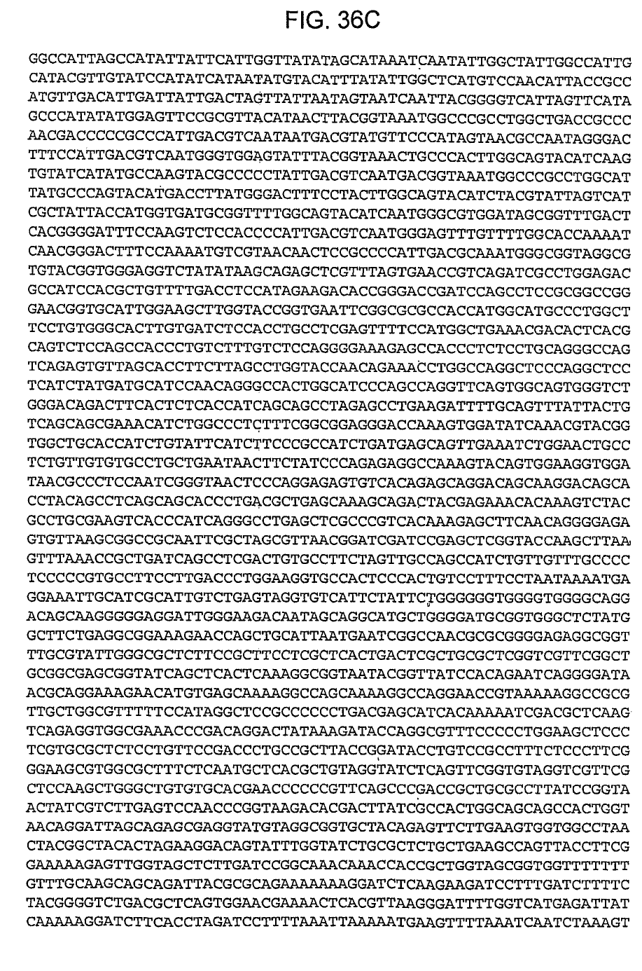

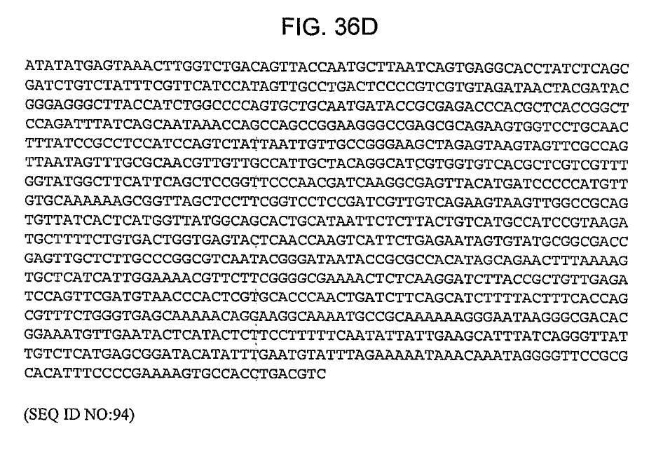

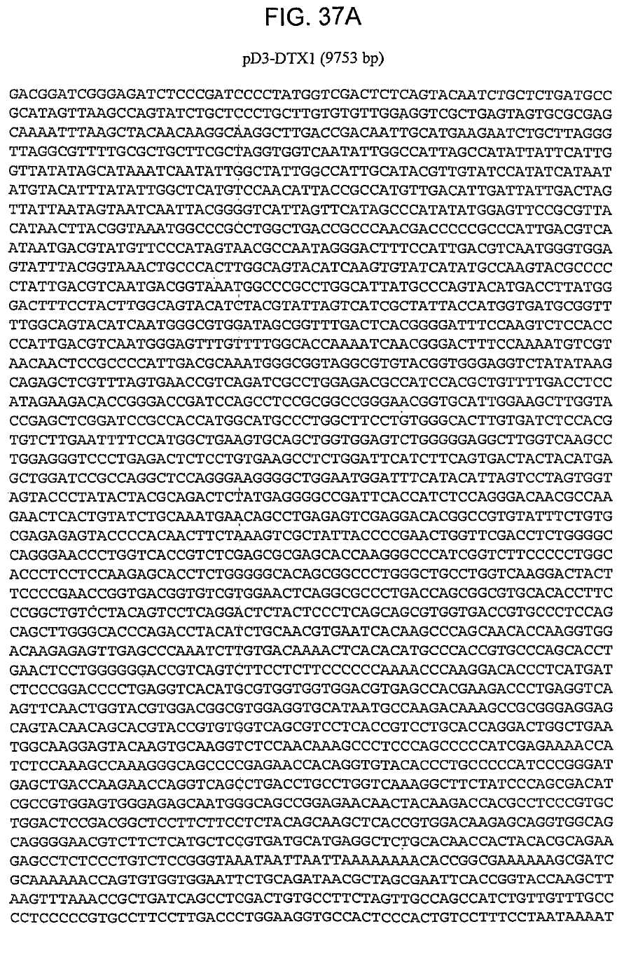

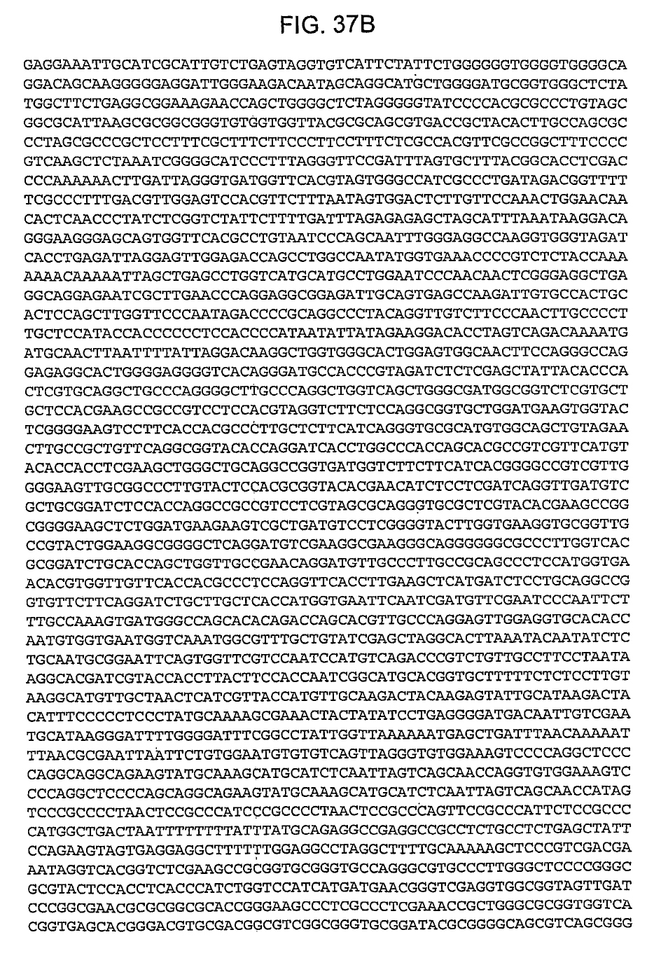

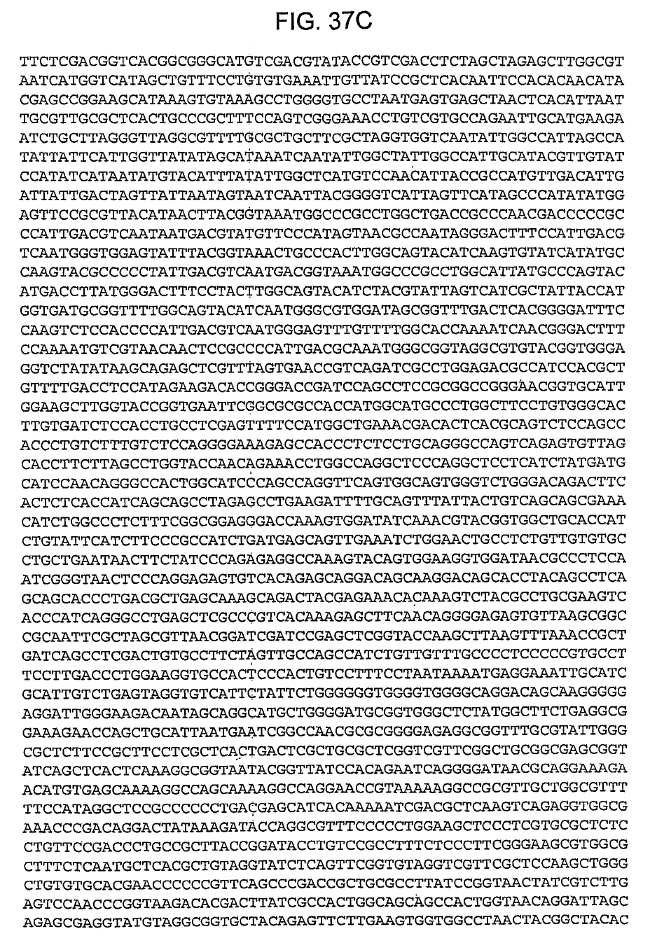

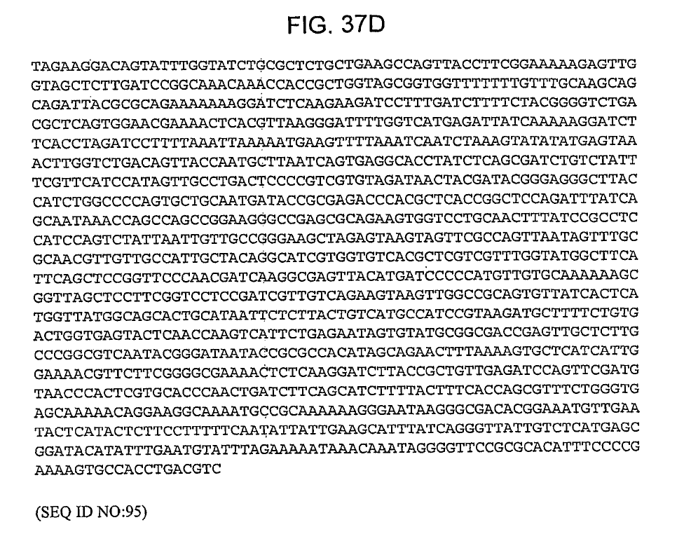

The functions of the target vector, the donor vector, and both integrase expression vectors were tested all at once by transfection and selection of PER.C6™ or DG44 cells as diagrammed in FIG. 11, before a large number of individual stable cell lines are generated. This experiment is only done once in the course of developing the methodology or as needed, for example, if variants of the target, donor, or integrase plasmids are constructed. Subsequently only the donor expression vectors which encode other proteins of interest are transiently transfected to test for expression of the protein of interest and confirm the donor vector is capable of expression.

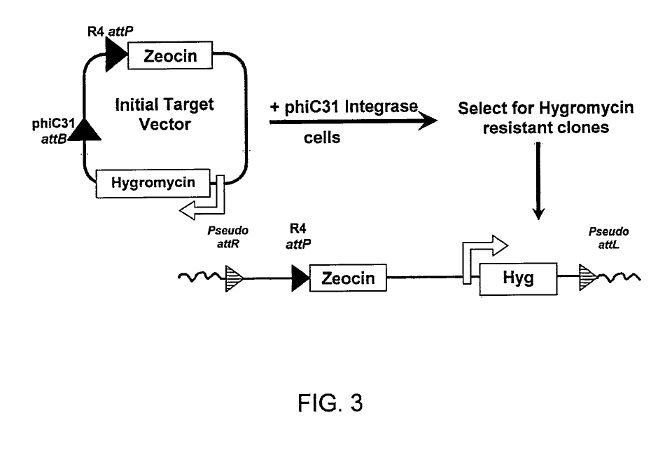

The target vector pR1 was co-transfected with a plasmid expressing the R4 integrase (pCMV-sre) into PER.C6™ or DG44 cells by lipofection using Lipofectamine 2000 CD (Invitrogen) according to the manufacturer's instructions. The cells were then incubated for forty eight hours to allow expression of the R4 integrase protein, which mediates site-specific integration between the R4 attB 295 site present on the target vector and pseudo R4 attP sites present in the chromosome (FIGS. 3 and 11). Colonies containing an integrated target vector were then selected in hygromycin containing media (e.g., DMEM, 10% fetal bovine sera, 10 mM MgCl₂ for PER.C6™ and F-12, 5% fetal bovine sera, 30 μM thymidine for DG44). Single, hygromycin resistant colonies were isolated and screened for puromycin sensitivity.

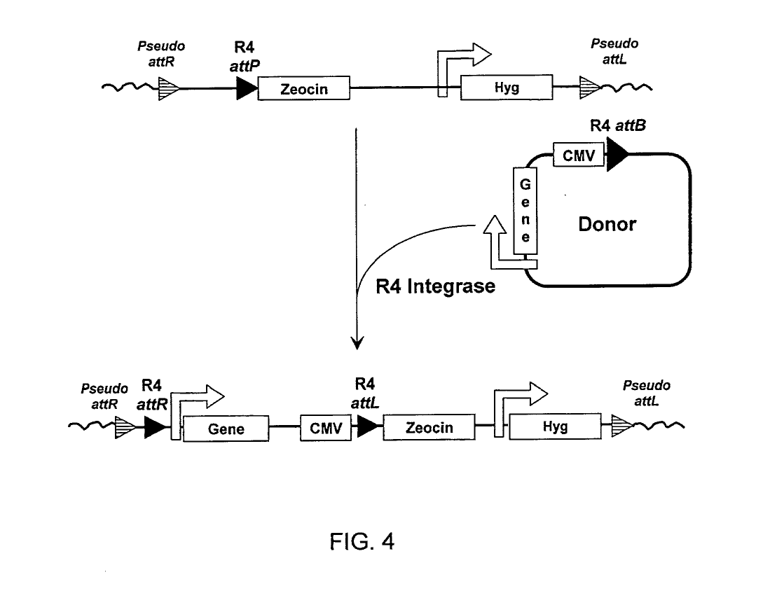

The hygromycin resistant, puromycin sensitive target vector clones were co-transfected again with a donor vector (e.g., pD1-DTX-1) containing the φC31 attB 285 AAA site and an expression cassette encoding genes of interest, such as the heavy and light chains of a human antibody specific for diphtheria toxin, and an expression plasmid encoding an altered φC31 integrase (e.g., pCS-M3J). The altered φC31 integrase protein mediates site-specific integration between the φC31 attB 285 AAA site present on the donor vector and the φC31 attP 103 site engineered into the chromosome of the cell line using the target vector (FIGS. 4 and 11).

A stable pool of puromycin-resistant cells is isolated as follows. Forty eight hours after the second transfection the regular cell growth media was replaced with cell growth media containing puromycin (1 μg/ml for PER.C6™, 10 μg/ml for DG44). The puromycin-containing media was changed every 2-3 days for 7 days (DG44 cells) or 14-21 days (PER.C6™ cells), or until the number of growing colonies became stable.

At this point all of the colonies were trypsinized and pooled. The cells were replated and allowed to attach for 24 hours. Selection for puromycin resistance was continued for a total of at least 21 days to allow for unintegrated expression vectors to be diluted. Then the expression level of the protein of interest (e.g., encoding an antibody) was assayed to confirm the function of both integrase expression vectors and the target vector and donor vectors. For measuring antibody expression an assay specific for human IgG (e.g., the Easy Titer IgG Assay, Pierce, Inc.) was used.

The target vector may not integrate or may integrate randomly at locations other than R4 pseudo attP sites. Even in these cases the donor vector can still integrate into the target vector to reconstitute a complete puromycin resistance gene. The number of puromycin colonies that would be expected to result from these events is much lower than those that occur as a result of integration of a donor vector into a target vector that was in turn integrated site-specifically using R4 integrase. This is because unintegrated vectors would be lost during the lengthy selection process. Random integration of a target vector will occur at a much lower frequency than site-specific integration mediated by the R4 integrase. To further document that protein expression levels measured in this experiment are primarily a result of the initial site-specific integration of the target vector, a control experiment is done in which the R4 integrase expression vector is omitted.

It is desirable to perform the puromycin resistance selection step to ensure it works because that step is the key to site-specifically integrating the donor expression vector. Integration of the φC31 attB site on the donor vector into the φC31 attP site on the target vector results in creation of a φC31 attL site, which in this specific example is 88 bases long. This additional sequence will be present in the 5′ untranslated region of the mRNA encoding puromycin resistance. Since the effect of this additional sequence on transcription, mRNA stability, translation, and hence ultimately on the level of puromycin resistance that can be achieved can not be predicted solely from nucleic acid sequences, the vectors should be tested as described above to ensure the reconstituted puromycin resistance cassette functions to a degree that allows efficient selection of cells in which the donor vector has integrated into the recipient vector.

### Example 2

### Construction of Protein-Expressing Cell Lines

The following protocol was followed for construction of protein-expressing cell lines. CHO/dhfr⁻ cells (e.g., DG44 cells and PER.C6™ cells) were transfected using Lipofectamine 2000 CD on 10 cm plates as follows:
1. The first transfection was done with 500 ng of the target vector pR1-DHFR and 5000 ng of the R4 integrase plasmid pCMV-sre (FIG. 11) per 10 cm plate.
2. The cells were grown for 48 hours in regular medium (Ham's F-12, 5% fetal bovine serum, 30 μM thymidine).
3. Then the cells were trypsinized and plated on 96-well plates in the selective medium, which was regular medium containing 400 μg/ml hygromycin B. Under these conditions, about 30 single cell clones grew on each of five 96-well plate.
4. Approximately 7-8 days after transfection when colonies are first visible by eye, the individual clones were trysinized and transferred to a minimal number of 96-well plates. A total of 165 clones were selected and consolidated on two 96-well plates.
5. The selected colonies were expanded onto a triplicate set of 96-well plates. One set was for maintenance. One set was frozen and stored in the vapor phase of liquid nitrogen. The third set was for the second transfection.
6. One set of CHO colonies was expanded to 24-well plates and co-transfected with 15 ng of pD1-DTX1-G418, the selectable donor expression vector, and 150 ng of pCS-M3J, the mutant φC31 integrase plasmid (FIG. 11).

7. The cells were grown for 48 hours in regular medium containing 400 μg/ml hygromycin B.
8. The cells were then grown in selective medium containing 10 μg/ml puromycin. After 7-21 days of selection variable numbers of colonies grew, depending on which parental attP cell line was transfected.
9. The colonies were then trypsinized and pooled. Half was plated in medium containing 10 μg/ml puromycin and half was plated in medium containing 10 μg/ml puromycin and 400 μg/ml G418.
10. The selective media was changed every 2-3 days until the wells were confluent. Pools of clones that grew in puromycin and G418 were expanded to 6 well plates and tested for IgG productivity (pg IgG produced/cell/day).
11. Out of 165 parental DHFR-target vector clones, 132 were puromycin sensitive and were used for the second transfection. Of these 96 produced puromycin resistant clones and were tested for IgG production. Out of 96 clones, 14 produced IgG at detectable levels.
12. The pool (2G7-G) with the highest level of expression (˜8 pg/cell/day) was grown in media selective for both the DHFR gene and the selectable donor expression vector (MEMα-, 7% dialyzed fetal bovine serum, 400 μg/ml G418) for 6 days and then plated at 1 cell per well on two 96-well plates in order to isolate clones.
13. A total of 56 clones were obtained and the IgG productivity of these was measured. The results are shown in FIGS. 28A and 28B. Three clones were identified that have average levels of productivity that are considered to be at the high end (i.e., >30 pg/cell/day).
14. Another pool (2H9-G), in which the DHFR gene was shown to be linked to the antibody genes by plasmid rescue methods, was subjected to DHFR gene amplification. The cells were grown in media selective for both the DHFR gene and the selectable donor expression vector (MEMα-, 7% dialyzed fetal bovine serum, 400 μg/ml G418). Then the DHFR gene was amplified by adding increasing amounts of methotrexate to the media. The starting concentration was 2 nM and the concentration was typically increased 2 to 3 fold about every 10-14 days.
15. The IgG productivities of the 2H9-G pool selected in various concentrations of methotrexate was measured and the results are shown in FIG. 29. At 200 nM methotrexate a dramatic increase in productivity was observed to a level equal to that of the highest expressing 2G7-G clones. However while it would take about 1 month to isolate the highest expressing 2G7-G clones using site specific integration, it would take about 4 months to isolate a high-expressing 2H9-G pool using gene amplification.

First Integration

In order to create a specific unique site for integration of a protein expression vector and to identify R4 Ψ attP sites in the genomes of cell lines that are suitable for high level, reproducible production of proteins either the target vector pR1 or the DHFR-target vector pR1-DHFR was integrated at a large number of different R4 Ψ attP sites in PER.C6™ and DG44 cells. The target vector or DHFR-target vector was mixed with the R4 integrase expression vector pCMV-sre and transfected into PER.C6™ and DG44 cells by lipofection according to the manufacturer's instructions. Liposomal reagents suitable for lipofection include Fugene 6 (Roche Applied Science), Lipofectamine 2000 CD (Invitrogen), and the like. The cells were incubated for forty eight hours to allow for expression of integrase and integration of either pR1 or pR1-DHFR into R4 Ψ attP sites to occur. The cell regular growth medium is then replaced with selective growth medium containing 100 ug/ml (for PER.C6™ cells) of 400 μg/ml (for DG44 cells) hygromycin B (Calbiochem). The cell growth medium was replaced every 2-3 days for 7-14 days or until a maximal number colonies are visible. A total of 100 colonies, which is estimated to represent about 50 different R4 Ψ attP sites, were picked and expanded for the second integration. Each cell clone isolated in this step is referred to as either a PER.C6™ attP cell line or a DG 44 attP cell line.

Sequences adjacent to integrated target vectors were determined to show they were integrated by an R4 integrase-mediated mechanism. To do this a “plasmid rescue” method was used that involves the following steps. Genomic DNA was prepared from target vector clones and digested with Afl III or Nsi I (New England Biolabs). These enzymes cut the target vector near the origin of replication but would not cut it at any other sites between the origin of replication and a W R4 attL site (see FIG. 12). Most importantly they also do not cut within the origin of replication and the ampicillin resistance gene, which are required for successful plasmid rescue in E. coli. The digested DNA was ligated at low concentration (˜10 ng/ml) and then electroporated into TOP10 cells (Invitrogen). Miniprep DNA was isolated from the resulting colonies and sequenced with a primer corresponding to the antisense strand of the puromycin coding region such that the sequence obtained would extend from the puromycin coding region through the φC31 attP site and then into the Ψ R4 attL site. As shown in FIG. 23 plasmids rescued from two target vector clones contained sequences up to the R4 att site core sequence and then extended into chromosomal DNA. The R4 att site core sequence was deleted in each case, as often occurs when serine integrases recombine a wild type att site with a Ψ att site.

Semi-random PCR methods can also be used to determine sequences at the junctions between target vectors and chromosomal DNA. For example the DNA Walking SpeedUp Kit (Seegene) can be used for this purpose. The “target-specific primers” would be located in the puromycin resistance gene to isolate a sequence containing the R4 Ψ attL site or in the HSK TK poly A area to isolate a sequence containing the R4 Ψ attR site

Alternatively “inverse PCR” methods can be used. In these methods genomic DNA is digested with a restriction enzyme that does not cut in the region of interest. The DNA is ligated to form circular DNA. Then the ligated DNA is amplified by the polymerase chain reaction using nested primers in known sequences. The orientation of the primers is inverted relative to what they would be in a normal PCR such that sequences across the point of ligation are amplified.

Prior to the second integration the attP cell lines are screened for puromycin sensitivity. A puromycin resistance selection is used to select the second integration step and thus it is useful to ensure the target vector or DHFR-target vector clones obtained in the first integration are puromycin sensitive. We have found that up to about 10% of the target vector or DHFR-target vector clones can be puromycin sensitive, depending on the cell line. Since the efficiency of integration is about 0.1-1% if a puromycin resistance clone was transfected it would be predicted that only 0.1-1% of the cells would express the proteins of interest and since the cells were already puromycin resistant it would not be possible to enrich for protein expressing cells. Another approach to circumvent this problem, besides screening target vector clones for puromycin sensitivity after the first transfection, would be to use a selectable donor expression vector in the second transfection.

Second Integration

In order to test the ability of each R4 Ψ attP site that the target vector integrated into in the first integration to allow high level protein expression, a second integration of a donor expression vector is done. A donor vector encoding an anti-diphtheria toxin antibody (pD1-DTX-1) was mixed with the φC31 mutant integrase expression vector (pCS-M3J) and transfected into each PER.C6™ attP or DG44 attP cell line generated in the first transfection by lipofection according to the manufacturer's instructions. Liposomal reagents suitable for lipofection include Fugene 6 (Roche Applied Science), Lipofectamine 2000 CD (Invitrogen), and the like. The cells were incubated for forty eight hours to allow for expression of the φC31 mutant integrase and integration of pD1-DTX-1 into the target vector to occur. The regular growth medium was then replaced with selective growth medium containing 1 μg/ml (for PER.C6™) or 10 μg/ml (for DG44) puromycin (Calbiochem). The cell growth medium containing puromycin was replaced every 2-3 days for 7-14 days or until a maximal number colonies are visible. The colonies arising from each transfection were trypsinized, expanded, frozen for liquid nitrogen vapor phase storage.

Sequences surrounding the junction of the target and donor expression vectors were determined to show they were recombined by a φC31 integrase-mediated mechanism. To do this a “plasmid rescue” method was used that involves the following steps. Genomic DNA was prepared from pools transfected with the donor and φC31 mutant integrase expression vectors. The DNA was digested with Tfi I (New England Biolabs). This enzyme cuts the expression vector within the heavy chain antibody gene and the target vector near the origin of replication but would not cut it at any other sites between these areas (see FIG. 13). Most importantly Tfi I does not cut within the origin of replication or the ampicillin resistance gene, which are required for successful plasmid rescue in E. coli. The digested DNA was ligated at low concentration (˜10 ng/ml) and then electroporated into TOP10 cells (Invitrogen). Miniprep DNA was isolated from the resulting colonies and sequenced with a primer corresponding to the antisense strand of the puromycin coding region such that the sequence obtained would extend from the puromycin coding region (from the target vector) through the φC31 attL88 site (junction between recombined target and donor vectors), and then into the bovine growth hormone polyadenylation signal (from the donor vector). As shown in FIG. 24A and FIG. 25A the sequence of plasmids rescued from DG44 and PER.C6™ cells was as predicted if φC31 integrase correctly integrated the donor expression vector into the target vector. The sequences surrounding the φC31 attR sites were determined in a similar manner and were also found to be exactly as predicted (FIG. 24B and FIG. 25B).

PCR-based methods were also developed to allow rapid determination of the types of integrations that might be present in clones or pools of clones. With regard to integration of the donor expression vector three types of integration are possible: random, target vector, or Ψ att site. To detect random integration, PCR primers specific for the φC31 attB site in the donor expression vector were designed. In most cases of random integration, the small (285 base pair) attB site would be intact, whereas if integration of the donor vector into a target vector or a Ψ att site had occurred the attB site would be disrupted. Genomic DNA from 6 pools of clones in which the donor vector had been integrated was prepared. One microgram of DNA was subjected to the polymerase chain reaction using primers 5′-CATCTCAATTAGTCAGCAACCATAGTC-3′ (SEQ ID NO:59) and 5′-AAGCTCTAGCTAGAGGTCGACGGTA-3′(SEQ ID NO:60) for 30 cycles and then 1% of that reaction DNA was subjected to the polymerase chain reaction using primers 5′-GTCGACGAAATAGGTCACGGTCTC-3′ (SEQ ID NO:61) and 5′-TACGTCGACATGCCCGCCGTGACC-3′ (SEQ ID NO:62) for 30 more cycles. The PCR products were separated on a 4% agarose gel and the results are shown in FIG. 26A. Evidence for random integration of the donor expression vector was absent from two pools (2G7, 2H10), but present in four pools (2B11, 2G11, 2H9G, 2H9P)

To detect the presence of integration into a target vector, a region containing the hybrid φC31 attR site was amplified by PCR directly on cells. Various numbers of trypsinized cells from the 2H9G pool were used. The 2H9G pool of cells was derived by transfecting a DG44 target vector (pR1-DHFR) clone (2H9) with a donor expression vector (pD1-DTX1-G418) and a φC31 mutant integrase vector (pCS-M3J). The cells were selected in puromycin for one month and then G418 for one month. Trypsinized cells were subjected to PCR amplification using primers 5′-TGCCCCGGGGCTTCACGTTTTCC-3′ (SEQ ID NO:64) and 5′-GCCCGCCGTGACCGTCGAGAAC-3′ (SEQ ID NO:65) for 30 cycles and then 1% of that reaction DNA was subjected to a subsequent round of PCR amplification using primers 5′-CAGGTCAGAAGCGGTTTTCGGGAG-3′ (SEQ ID NO:63) and 5′-CCGCTGACGCTGCCCCGCGTATC-3′ (SEQ ID NO:66) for 30 more cycles. The PCR products were separated on a 4% agarose gel and the results are shown in FIG. 26B. A specific signal of the correct size was amplified when 10², 10³, or 10⁴ cells were used.

Semi-random PCR methods can be used to determine whether a donor vector has integrated into a Ψ φC31 att site. For example the DNA Walking SpeedUp Kit (Seegene) can be used for this purpose. Alternatively the inverse PCR method can be used.

Antibody production levels were tesed as follows. A known number of cells was plated in a 6 well dish in either MEMa-media (Invitrogen) with 7% dialyzed fetal bovine sera (Invitrogen) for CHO DHFR— cells or DMEM (Invitrogen), 10% fetal bovine sera (JRH), 10 mM MgCl₂ for PER.C6™ cells. The cells were allowed to grow for 1-4 days. The media was harvested and at the same time the final number of cells was determined.

The cell number was determined using a hemocytometer. Alternatively, a MTT-based assay kit (Cell Titer 96 kit, Promega) or similar kits can be used to determine the number of cells on the plate. Instruments such as the ViaCount Assay (Guava) that can measure the number of adherent cells on a plate are also available.

The concentration of IgG in the media was determined using the Easy-Titer Human Ig (H+L) Assay Kit (Pierce) that specifically measures all classes of human IgG. The specific productivity (picograms antibody/cell/day) was calculated from the following equation:

pg/ml antibody X ml of media harvested (Final cell number+initial cell number)/2 Number of days antibody was produced

The results of screening 100 PER.C6™ attP cell lines and 100 DG44 attP cell lines are shown in FIG. 27A and FIG. 27B, respectively. Sixteen DG44 attP cell lines gave rise to pools of puromycin resistant clones with detectable expression and the best pool produced about 8 pg antibody/cell/day (FIG. 27A). Seventeen PER.C6™ attP cell lines gave rise to pools of puromycin resistant clones with detectable expression and the best pool produced about 4 pg antibody/cell/day (FIG. 27B).

Often pools of clones will contain cells that vary greatly in terms of protein expression. Therefore, we subcloned high producing pools in order to identify specific cell lines within the pools that provide a high level of protein expression. The pool derived from transfection of DG44 attP cell lines with the donor expression vector which exhibited the highest expression level (2G7) was subsequently cloned by limiting dilution on 96-well plates and assayed for antibody productivity as described above. The results are shown in FIG. 28. Within the pool, which produced 7.6 pg/cell/day, are clones that vary in productivity from 0.2 to 38 pg/cell/day. Three clones produced more than 30 pg/cell/day.

Cells that express very high levels of proteins are often at a growth disadvantage and therefore may be lost or underrepresented when expanded as described above as part of a pool. A method to circumvent this problem is as follows. After transfection with the donor expression vector and the φC31 integrase vector, the cells are incubated 48 hours to allow integration to occur. Then the transfected cells are trypsinized and plated on 96 well plates such that single colonies will grow in about 30% of the wells. The number of transfected cells that are plated per well depends on the plating efficiency and the donor vector integration efficiency. In general to obtain the maximum number of single cell clones on a 96 well plate about 0.3 cells with 100% viability are plated per well. Thus, for example, if the plating efficiency of a cell is 50% and 0.1% of the cells undergo an integration event that results in a puromycin resistant cell one would plate 0.3/0.5/0.001=6000 cells per well after transfection in order to obtain clones. If the integration efficiency is very high one may need to transfect fewer cells.

The parental PER.C6™ attP or DG44 attP cell lines that result in the highest number of clones with the highest protein expression levels are chosen to be used as the attP cell lines for integrating other donor expression vectors and producing other proteins at high levels. Those cell lines are used repeatedly and only a small number (<50) of clones are generated and screened to identify those with the highest expression levels. This scheme will work for expression of a variety of proteins, showing that the ability to achieve high expression levels by integration at one site is not specific to antibody expression. This method saves a substantial amount of time compared to methods that are currently used which can require screening hundreds or thousands of clones every time a different protein is produced. In addition, by integrating expression cassettes at the same loci each time the stability of the genes and the expression of proteins encoded by those genes is more predictable compared to methods that are currently used in which gene and protein expression stability is often highly variable, and as a result can require screening of additional clones and time-consuming assays to identify those cell lines that are stable enough to be useful. This method also eliminates gene amplification methods which often are used to boost expression if a cell line having a high level of protein expression is not obtained. Such gene amplification methods, such as those utilizing the dihydrofolate reductase gene or the glutamine synthetase gene, often take 3-6 months to achieve high expression levels and in many cases the expression may not be stable.

Several features of the chromosomal configuration that results when the donor vector is integrated into the target vector are worth noting (FIGS. 11-13). First, all promoters are in the same or opposing orientations to avoid generating antisense transcripts and siRNA that might reduce gene expression. Second, a dual CMV promoter configuration equalizes expression of the heavy and light chains of an antibody. This is important because often when there is an imbalance in the expression of the heavy or light chain proper assembly does not occur or they are degraded. Third, the φC31 attB 285 AAA and φC31 attP 103 sites were designed so that when they recombine a short 88 base long φC31 attL site, containing no upstream translation start codons, results. The short length of φC31 attL 88, which is present in the 5′ UTR of the mRNA encoding puromycin resistance, minimizes interference with expression of puromycin resistance.

Another exemplar configuration includes one in which the φC31 attL site ends up being located in an intron. To generate this configuration the donor vector is constructed to contain (in order) a promoter, the N-terminal half of the coding region of a drug resistance gene, and the 5′ half of an intron preceding a φC31 attB site. The target vector is then constructed to contain (in order) the 3′ half of an intron, the C-terminal half of the coding region of a drug resistance gene, and a poly A signal following a φC31 attP site. After integration of such a donor vector into such a target vector a fully functional drug resistance expression cassette is reconstituted which consists of a promoter, the complete coding region of a drug resistance gene, and a poly A signal. The φC31 attL site will be present in the intron.

Extensive information is available about which nucleotide sequences in an intron are required for proper splicing to occur. For example, sequences near the 5′ and 3′ exon/intron junctions and a polypyrimidine tract that is typically located about 30 bases 5′ to the 3′ end of the intron are required for efficient splicing to occur. Therefore, in configurations described above the attB in the donor vector and attP in the target vector are placed in the middle of an intron at least 100 bases from either end of the intron so that the resulting attL site will be in the middle of the intron far from any nucleotide sequences that are critical for proper splicing to occur. This will ensure that the resulting attL site is very unlikely to interfere with splicing. In addition, the intron can be long (>1 kbp) to further minimize the potential that the attL site will interfere with splicing.

Methods for Cell Line Characterization

Several procedures can be performed to characterize the gene cassette that is present in and the proteins that are produced by cell lines derived using the methods described above. The gene cassette is characterized to determine where the cassette integrated and to ensure the predicted structure is present and stable over time. The protein that is being produced by the cell line is also characterized to ensure it is present, active, and that high-level production is stable over time.

To characterize the number of integration sites and their location a number of methods are available. In some embodiments, Fluorescence in situ hybridization (FISH) is used to determine the number of integration sites in the entire genome. The location of integration sites is determined by isolating and sequencing chromosomal DNA that flanks the integrated cassette and compared to the sequence of the entire human genome (see for example Chalberg, et al., 2006).

The entire integrated cassette is isolated in two fragments by a “plasmid rescue” method every month so that the cassette is archived in case it is desirable to do a retrospective analysis. In short, plasmid rescue involves preparing genomic DNA from cell lines, digesting it with restriction enzymes that cut once in the integrated cassette and once in genomic DNA such that the DNA fragment will have an origin of replication and a selectable marker suitable for maintenance and selection in E. coli. The digested DNA is ligated and used to transform E. coli. Any DNA that contains an E. coli origin of replication (e.g., ColE1) and a selectable marker (e.g., ampicillin resistance) replicates and thus is “rescued”. The DNA cassette that results from integration of the target vector into a Ψ R4 attP site and then subsequently integration of the donor vector pD1 into the integrated target vector will have two E. coli origins of replication and two selectable markers. Several restriction enzymes cut between these sequences once and thus enable rescue of DNAs containing the target and donor vectors separately. By using this method the expression cassette integrity and stability over time can be determined. For example, the entire cassette (˜14 kbp) can be sequenced to confirm it has the intended sequence and arrangement of DNA elements.

If the restriction site in the chromosomal DNA is too far from the integrated cassette to generate a DNA small enough to be replicated in E. coli, plasmid rescue may be unsuccessful. In such embodiments, the polymerase chain reaction is used to analyze the integrated cassette. Several enzymes and conditions are available such that the entire ˜14 kbp integrated cassette can be amplified and stored as-is with no further cloning. If it is desirable to obtain the sequences of flanking chromosomal DNA a number of methods are available, such as inverse PCR or approaches that use random primers to amplify the flanking chromosomal sequences.

In addition to determining which genes are present it is also desirable to ensure that the integrase vectors have not integrated into the genome. This is because persistent expression of integrase could lead to instability of the integrated target and donor vector cassettes or instability of chromosomal DNA by mediating recombination between Ψ att sites present in the genome. Stable integrase vectors have been observed after a transient transfection, but are rare. However, in some embodiments it may be desirable to rule out the presence of integrase vectors in the cell lines. Any suitable methods for detecting the presence or absence of specific nucleic acids, such as Southern blotting or the polymerase chain reaction, can be used to determine if integrase vectors are present. Alternatively methods such as Western blotting or ELISA, which detect the presence of an integrase protein, can be used.

Characterization of Protein Production

In addition to characterization of the integrated gene cassettes, the quality, stability, and level of protein production (e.g., antibody production) is also characterized. Initially, a large number of pooled cell lines (>100) from the second integration were screened for protein production in a 96-well plate. A variety of suitable methods for antibody screening can be used. For example, an ELISA is used to measure the total amount of antibody present. If the level of antibody that is made is produced at a suitable level, SDS-polyacrylamide gel can also be used to screen production levels. If the cells are grown in serum-free media, it is possible to load cell culture supernatants directly on an SDS-PAGE gel. If the cells are grown in serum-containing media the antibody can be detected specifically and quantitated by, for example, Western blotting or ELISA.

Specific Binding Activity of Antibody Produced by Cells

DG44 or PER.C6™ were transfected with pD1-DTX1 (using Lipofectamine 2000 CD as described elsewhere). Twenty four hours after transfection the media was harvested. Total IgG was determined using an Easy-Titer (H+L) IgG assay kit (as described in other places in patent.) Anti-diphtheria toxin IgG was determined using a Diphtheria IgG ELISA kit (IBL Hamburg) exactly according to the manufacturer's instructions.

FIG. 31 shows the specific binding activity of anti-diphtheria toxin antibody expressed in DG44 cells or PER.C6™ cells. The antibody produced from each cell has the same specific binding activity. In addition, the results show that the antibody from both cell lines has the correct antigen specificity and that ˜250 mg of this antibody would be needed for a typical 10,000 IU dose.

Biological Activity of Antibody Produced by Cells

A neutralizing assay can also be used to measure functional activity of an antibody. For example anthrax toxin and other toxins such as diphtheria toxin kill cultured cells. Therefore the activity of an anti-diphtheria toxin antibody can be determined by measuring its ability to neutralize the cell killing properties of purified diphtheria toxin. The ratio of functional activity to total protein (specific activity) is a useful measure the level of active antibody or other secreted protein a particular cell line produces.

The neutralizing activity of the anti-diphtheria toxin antibody produced from DG44 or PER.C6™ was determined and compared to antibody from the D2.2 cell line, from which the anti-diphtheria toxin antibody genes were cloned. The antibody from DG44 or PER.C6™ was generated by transient transfection of cells using Lipofectamine 2000 CD as described elsewhere. The amount of antibody present in supernatants from D2.2 cells or the transfected DG44 and PER.C6™ cells was determined by ELISA using pure diphtheria toxin as the antigen. Then various amounts of antibodies were added to 10 ng/ml diphtheria toxin. After a 15 min incubation at 37° C. the antibody/toxin mixtures were added to Jurkat cells, which are sensitive to killing by diphtheria toxin. Cell division was measured by ³H-thymidine incorporation. The results are shown in FIG. 32. Control cells which were treated with toxin only and no antibody die as indicated by the lack of significant ³H-thymidine incorporation. Cells treated with increasing amounts of anti-diphtheria toxin antibody produced by D2.2, DG44, or PER.C6™ cells survived. The EC₅₀ for protecting Jurkat cells from killing by diphtheria toxin was 5, 8, and 11 ng/ml for the anti-diphtheria toxin antibodies produced by D2.2, DG44, or PER.C6™ cells, respectively.

About ten cell lines that produce the highest levels of antibody on a small scale are adapted to serum-free suspension culture at a larger scale (e.g., 100 ml-1 liter). Several clones are adapted since some may not adapt, grow fast, or retain high-level antibody expression levels. After adaptation of the cell lines to suspension culture antibody production levels are tested again. Exemplary antibody production at a laboratory scale is about 10-100 mg/L of media per day or approximately 10-100 pg/cell/day assuming a maximal cell density of 1×10⁹ cells per liter.

A variety of methods have been described for large scale human IgG antibody purification. Typically at least three chromatography resins are used. A Protein A column is used as a first affinity step to capture the IgG by binding to its Fc region. The second column is designed to remove endotoxin, remaining cellular proteins, and any protein A that leached from the first column. Exemplary resins include, hydroxyapatite, hydrophobic interaction, or cationic exchange resins that can be used for the second chromatography step. An anion exchange column is used as the third step to remove DNA.

About 100 mg of antibody is purified and tested in an appropriate activity assay. For anti-diphtheria toxin antibodies an appropriate in vivo assay is a skin test done in guinea pigs. The antibody is mixed with purified diphtheria toxin and injected into the skin. Toxin that is not neutralized results in an inflammatory response. For anti-diphtheria toxin antibodies an appropriate in vitro assay is one using Vero cells. As little as one molecule of diphtheria toxin (Sigma) is thought to be capable of killing cells via a covalent ADP-ribosylation of the elongation factor-2 (EF-2) ribosomal accessory protein. As a result all protein synthesis in the cell is inhibited and the cells die. Thus any assay that measures cell viability or cell metabolism such as an MTT-based assay is used to determine the titer of the antibody against a given amount of purified diphtheria toxin. Such assays are done every month for 12 months to establish a shelf life and study the stability of the purified antibody.

A SDS-polyacrylamide gel is used to assess some basic features of the antibody. For example SDS gel electrophoresis of a reduced antibody sample can be used to confirm the amount, purity, and correct molecular weight of the heavy (˜50 kDal) and light chains (˜25 kDal), but more importantly to confirm that the ratio of heavy to light chain is about 1:1. SDS gel electrophoresis of a denatured but non-reduced sample is used to determine whether the antibody is primarily monomeric or multimeric. This is important because the presence of aggregated antibody may indicate production or purification problems. Aggregated antibodies can have undesirable effects, such as kidney toxicity, when used as human therapeutics. Finally, aggregated antibodies are also often inactive with regard to their desired biological activity. Other bioanalytical methods can also be used to assess the aggregation state of an antibody including light scattering or gel filtration.

### Example 3

### CHO Cell Line for Protein Production Using a Selectable Donor Expression Vector

We found that transfection of DG44 pR1-DHFR cell clones with the φC31 mutant integrase expression vector pCS-M3J alone could result in puromycin resistant cells without transfecting the donor expression vector. This appears to be the result of φC31 integrase-mediated rearrangements of chromosomal DNA into the integrated pR1-DHFR plasmid in areas 5′ to the puromycin resistance gene. Such translocated chromosomal DNAs may contain promoters that drive expression of puromycin resistance. In some experiments the number of these events was up to 30% of the number of desired integration events in which the donor expression vector integrated into the target vector.

One method to circumvent this problem was to have a complete functional drug resistance gene, such as one encoding resistance to G418, on the donor expression vector. After transfection of target vector clones with a G418 gene-containing donor expression vector and the φC31 integrase vector, followed by selection for puromycin there will be two classes of integrants. In one class recombination of the donor expression vector into wild type att P sites in the target vector will have occurred and in another class rearrangements of chromosomal DNA into the target vector will have occurred. However if a G418 selection is applied after the puromycin selection only the recombinants with a complete donor expression vector will remain. Cells in which rearrangements of chromosomal DNA into the target vector has occurred will not contain the G418-donor expression vector and will be eliminated.

Note that the order of the drug resistance selections is important. If the G418 selection was done first, then cells with the G418-donor expression vector integrated randomly, into the target vector, and into Ψ att sites might be obtained. Then if a puromycin selection was done subsequently the cells with random or Ψ att site integrations would be eliminated, but chromosomal rearrangements into the target vector may still occur such as in the cells in which donor expression vector integration into the target vector had not occurred. For similar reasons it is undesirable to do the puromycin and G418 selections simultaneously.

To determine if doing a G418 selection after the puromycin selection was beneficial, pD1-DTX1-G418 was transfected into DG44 R1-DHFR clones 1A1, 2B11, 2E8, 2G7, 2H1, 2H9 as described in Example 2. Two days after transfection the cells were selected in 10 μg/mlpuromycin for 7 days. Then the colonies were split into either growth media containing 10 μg/mlpuromycin only or both 10 μg/ml puromycin and 400 μg/ml G418. Selection under these conditions continued for 21 days. Then the media was assayed for antibody production. The results of these assays are shown in Table 1. The G418 selection increased the specific productivity by 30 to 73-fold in 4 cases and had no effect in two cases. Whether or not G418 selection had an effect may depend on the efficiency of donor expression vector integration in each target vector clone, and also on the frequency of expression vector-independent events that result in puromycin resistance.

TABLE 1
Effect of using a selectable donor expression
vector on protein production

| | | | Production |
|---|---|---|---|
| Target | IgG production | IgG production | ratio (with G418 |
| vector clone | (after puromycin | (after puromycin | selection/witout |
| transfected | and G418 selection) | selection only) | G418 selection) |
| 1A1 | 15 ng/ml | 19 ng/ml | 0.8 |
| 2B11 | 1795 ng/ml | 56 ng/ml | 32 |
| 2E8 | 585 ng/ml | 10 ng/ml | 59 |
| 2G7 | 1017 ng/ml | 34 ng/ml | 30 |
| 2H1 | 815 ng/ml | 658 ng/ml | 1.2 |
| 2H9 | 1688 ng/ml | 26 ng/ml | 73 |

Complete drug resistance genes, other than one encoding resistance to G418, can be optionally incorporated into a selectable donor expression vector. The only limitation is that it must be different from the one used to select target vector inetgration (e.g., hygromycin resistance), select donor vector integration (e.g., puromycin resistance) or amplify the copy number of the target vector (e.g, dihydrofolate reductase). Thus, for example, genes encoding resistance to zeocin or blasticidin could be utilized.

Another benefit of using a selectable donor expression vector is that after φC31-mediated integration of a selectable donor expression vector into a target vector, such as pR1-DHFR, the selectable gene will be located between the coding regions of the antibody heavy and light chains. Hence continuous selection will prevent homologous recombination between repeated elements of the expression vector (e.g., promoter, signal sequence, poly adenylation signal) which could result in deletion of either the heavy or light chain coding regions.

### Example 4

### Engineered CHO Cell Line for High Yield Protein Production

The method of culturing and transfecting CHO cells will follow the procedure as described in Thyagarajan et al., Methods Mol. Bio., 308:99-106 (2005). Briefly, CHO/dhfr⁻ cells (e.g., DG44 cells) will be transfected using Fugene 6 in a 24 well plate. The following protocol is followed:
1. The first transfection is done with the target vector and φC31 integrase plasmid (FIG. 3).
2. 24 hours after transfection, the cells are transferred to 100-mm dishes.
3. 48 hours after the transfection, the cells are selected for hygromycin resistant clones.
4. Approximately 12-14 days after transfection when well-formed colonies appear, the individual clones are picked and transferred to a 24-well plate. From previous experience with using φC31 integrase, only 30-50 clones need to be screened to obtain high-expression clones.
5. The selected colonies will be maintained in two sets of 24-well plates. One set is for maintenance. The other set is for screening.
6. The screening set of CHO colonies in the 24-well plates is co-transfected with the donor vector expressing a reporter gene (for example, CIP, GFP or luciferase), and the R4 integrase plasmid (FIG. 4).
7. 48 hours after the second transfection, the non-selective medium is removed from the plates and medium containing zeocin is applied several times for about 2 weeks.
8. Cells are then harvested for appropriate reporter gene assays.
9. 3-5 clones are selected that express the highest levels of reporter gene, and the corresponding clones are expanded from the maintenance set.
10. The resultant cell lines, containing an R4 integrase phage attachment site (attP), are referred to as CHO—R4attP cells.
   Testing the CHO—R4attP Cell Line

A SARS or anthrax antibody is used to test the CHO—R4attP cell line. Most of the SARS and anthrax antibodies are IgG1. The V_{H} and V_{L} variable regions of the antibodies are cloned and then assembled in a vector that contains IgG1 constant regions to produce full-length antibodies. The cDNAs for the heavy chain and the light chain can either be cloned into two separate donor plasmids or into a single donor plasmid in tandem driven by either two identical or two different promoters. An advantage of using a phage integrase is that there is no size limitation on the gene of interest. Both a two-plasmid system and a one-plasmid system will be used to express the full length antibodies.

The expression of monoclonal antibodies at research scale has been extensively described (Wurm et al., Nat Biotechnol 22, 1393-8 (2004); Andersen et al., Curr Opin Biotechnol 13, 117-23 (2002); Wirth et al., Gene 73, 419-26 (1988); Kim et al., Biotechnol Bioeng 58, 73-84 (1998); Gandor et al., FEBS Lett 377, 290-4 (1995); and Kito et al., Appl Microbiol Biotechnol 60, 442-8 (2002)). These common procedures are followed with respect to the CHO—R4attP cell line. The serum-free medium and cell culture process is developed to optimize the antibody production for large-scale fermentation.

The parental cell line, a subclone of CHO/dhfr⁻, is selected to produce protein with a high yield of 30-50 pg/cell/day in serum-free medium. The expected production rate using the engineered CHO—R4 attP cell line will be about at least 30 pg/cell/day in serum-free medium. Once the cell line and the donor vector are developed, any antibody gene of interest can be conveniently cloned into the expression cassette of the donor vector (FIG. 2). Since selecting for high level expression clones only requires the screening of 30-50 colonies, a stable cell line that expresses high levels of an antibody can be rapidly generated in a cost-effective manner.

Characterization of the CHO—R4attP Cell Line

The memorandum “Points to Consider in the Characterization of Cell Lines Used to Produce Biologicals (1993)” published by the Center for Biologics Evaluation and Research (CBER) of the FDA is followed to characterize the CHO—R4attP cell line.

In addition, the R4 attP integration site is fully characterized, for example with regard to the number of copies and locus of the integration, by conventional methods, for example FISH, Southern blots, PCR, and DNA sequencing. Since the future integration of a gene of interest will be specifically targeted to the R4 attP site that has been previously engineered into the chromosome, characterization of the integration site of each individual gene of interest is trivial. Consequently, the future characterization of stable cell lines that express the gene of interest is significantly simplified, saving time and cost.

### Example 5

### Engineered DHFR-Amplifiable CHO Cell Line for High Yield Protein Production

The DHFR-amplification system is widely used in CHO expression systems in order to increase the copy number of a DHFR associated expression cassette. The expression system utilizes dihydrofolate reductase (DHFR) deficient CHO host cells in conjunction with a transfected DHFR gene as a selectable marker. The system amplifies genes and sequences linked to DHFR, which leads to enhanced levels of protein expression (Wurm et al., Nat Biotechnol 22, 1393-8 (2004)). Transfected cells develop resistance to methotrexate (MTX), a DHFR inhibitor, through amplification of the DHFR gene and up to 100-10,000 kilobases of the surrounding region (Coquelle et al., Cell 89, 215-25 (1997); and Stark et al., Cell 57, 901-8 (1989)). After 2-3 weeks of exposure to MTX, the majority of cells die. However, the surviving cells often contain several hundred to a few thousand copies of the integrated plasmid (Wurm et al., Ann N Y Acad Sci 782, 70-8 (1996); and Wurm et al., Biologicals 22, 95-102 (1994)). Most of the “amplified” cells produce up to 10- to 20-fold more recombinant proteins (Wirth et al., Gene 73, 419-26 (1988)). Several cycles of gene amplification are often performed and typically the concentration of methotrexate is increased 3-5 fold after each gene amplification cycle. Three alternative options are tested for optimal DHFR-amplification.

To test whether DHFR amplification of the gene of interest would allow for increased protein expression, the DHFR gene was placed on the target vector. A schematic of a target vector including a DHFR gene is provided in FIG. 15. The sequence of the resulting vector is provided in FIGS. 35A-35C. FIG. 29 shows expression of an antibody (pg/cell/day) from a pool of cells in which a donor expression vector was site-specifically integrated into a DHFR-target vector and cell populations were then exposed to increasing concentrations of methotrexate.

There are at least three advantages of linking the DHFR gene with the R4 attP site on the target vector. First, after DHFR amplification, the chromosome will also have multiple copies of the R4 attP site. After the donor vector is transfected into the CHO—R4attP (DHFR) cell line, the gene-of-interest may be integrated into multiple receiving R4 attP sites, mediated by the R4 integrase. Second, if the previously amplified CHO—R4attP (DHFR) cell line already has the capacity to express a sufficiently high level of the gene-of-interest, a second DHFR amplification may not be required after the gene-of-interest is transfected, thus saving significant time and effort. Third, since the CHO—R4attP (DHFR) cell line will have been well characterized, after integration of the gene-of-interest from the donor vector, the expression cell line producing the gene-of-interest may not need another lengthy DHFR amplification and further characterization, saving a significant amount of time and cost.

In a second example, the DHFR gene is present on the donor vector. A schematic of the donor vector including a DHFR gene is provided in FIG. 6. In a third example, the DHFR gene is present on the target vector (FIG. 5) and on the donor vector (FIG. 6). After DHFR amplification, the engineered CHO—R4attP (DHFR) cell line is expected to produce a yield well above 30 pg protein/cell/day in serum-free medium.

### Example 6

### Engineered CHO Cell Line for High Yield Protein Production with Enhanced Translation Using an IRES

The possibility and necessity of using an optimized IRES-element together with φC31 integrase to further increase the expression level is also tested. The optimized IRES-element is cloned into the donor vector, upstream of the coding region for the protein of interest and downstream of the transcription start site (FIG. 7). This IRES-element will significantly increase protein production by enhancing the translation efficiency of the target mRNA (Chappell et al., J Biol Chem 278, 33793-800 (2003); Owens et al., Proc Natl Acad Sci USA 98, 1471-6 (2001); and Chappell et al., (2000) Proc. Natl. Acad. Sci. U.S.A., 97, 1536-1541).

To obtain large quantities of therapeutic proteins and antibodies, overexpressing cell lines are developed that use novel translation-based technologies that are capable of much higher levels of protein production than is possible using traditional transcription based methods which increase the amount of target gene mRNA, e.g. through the use of strong promoters, chromosomal duplication, and selection of high expressing cell lines.

Translational enhancers have been developed recently using short RNA sequences that function as internal ribosome entry sites (IRESes) that recruit the translation machinery and facilitate translation initiation. Although the activity of individual IRES-elements is relatively weak, it was shown that IRES activity could be increased synergistically when particular IRES elements were linked together (Owens et al., Proc Natl Acad Sci U S A 98, 1471-6 (2001); and Chappell et al., (2000) Proc. Natl. Acad. Sci. U.S.A., 97, 1536-1541). In these studies, synthetic IRESes were tested in the intercistronic region of dicistronic mRNAs for their ability to enhance the translation of the second cistron. However, it was recently shown that one of these IRESes could also function as a potent translational enhancer when placed in the 5′ leader of a monocistronic mRNA. This synthetic IRES contained multiple linked copies of a 9-nt IRES-module from the 5′ leader of the Gtx homeodomain mRNA.

A goal is to identify IRES elements that function efficiently in CHO cells and use these individual elements to generate synthetic translational enhancers that function efficiently in CHO cells. Translational enhancers are also developed that function efficiently in human-hybrid and human cell lines that are used for large scale production.

Individual IRES elements that function efficiently in these cell lines are obtained using a selection methodology in which a cassette containing 18 random nucleotides is cloned into a selection vector and transfected into the cell line of interest (Owens et al., Proc Natl Acad Sci USA 98, 1471-6 (2001)). Selection experiments are performed using a GFP/CFP dicistronic retroviral vector. Cells containing active IRES elements are selected by FACS. Selected sequences are recovered and retested in a Renilla/Photinus (RPh) dual luciferase vector to show the IRES functions in another context and is not dependent on or influenced by sequences present in the GFP/CFP vectors used to select them. Various IRES elements are tested for their ability to synergize activity by linking together multiple copies of the same or different IRES-elements. Combinations of elements that show enhanced IRES activity are tested for their ability to function as translational enhancers in the 5′ leader of a monocistronic reporter RNA.

The synthetic translational enhancers that are generated are then tested in the 5′ leaders of mRNAs encoding therapeutic proteins or antibodies to determine which enhancer/gene combinations function most efficiently. Once particularly efficient combinations are identified, constructs are tested in scaled up culture conditions and further optimized if necessary to maximize antibody production.

### Example 7

### Engineered CHO Cell Line for High Yield Inducible Protein Production

Cell lines suitable for scale-up and manufacturing must have the combined capacity for fast growth and high specific-productivity. Due to the high expression level of the expression vector, the production cells might have difficulties growing when expressing high levels of foreign proteins, or the foreign proteins may aggregate during a prolonged growth phase. If this difficulty is encountered, an on-off switch is added to the donor vector to provide for inducible expression of the gene of interest. As such, the element would function to turn off the transgene expression during cell growth and would only turn on the expression when cells have grown to a critical amount and are ready for protein production. These switches are actuated by ligands that interact with an appropriate receptor system that conditionally interferes with or activates transcription. Several proprietary switches have been developed for gene therapy studies and can be used in the production system envisioned, including, but not limited to, the ARGENT system, the GENE SWITCH system, riboswitches, zinc finger proteins, ecdysone receptor-based systems, and the like. In addition, tetracycline-inducible and gas-inducible systems can also be utilized (Weber et al., Nat Biotechnol 22, 1440-4 (2004); and Weber et al., Metab Eng 7, 174-81 (2005)).

### Example 8

### Engineered PER.C6™ Cell Line for High Yield Protein Production

The method of culturing and transfecting PER.C6™ cells will follow the procedure as described in Thyagarajan et al., Methods Mol. Bio., 308:99-106 (2005). Briefly, PER.C6™ cells will be transfected using Fugene 6 in a 24 well plate. The following protocol is followed:
1. The first transfection is done with the target vector and φC31 integrase plasmid (FIG. 3).
2. 24 hours after transfection, the cells are transferred to 100-mm dishes.
3. 48 hours after the transfection, the cells are selected for hygromycin resistant clones.
4. Approximately 21 days after transfection when well-formed colonies appear, the individual clones are picked and transferred to a 24-well plate. From previous experience using φC31 integrase, only 30-50 clones need to be screened to obtain high-expression clones.
5. The selected colonies are then maintained in two sets of 24-well plates. One set is for maintenance. The other set is for screening.
6. The screening set of PER.C6™ colonies in the 24-well plates is co-transfected with the donor vector expressing a reporter gene (for example, SEAP, CIP, GFP or luciferase), and the R4 integrase plasmid (FIG. 4)
7. 48 hours after the second transfection, the non-selective medium is removed from the plates and medium containing zeocin is applied several times for about 3 weeks.
8. The cells are then harvested for appropriate reporter gene assays.
9. 3-5 clones that express the highest levels of reporter gene are selected and the corresponding clones from the maintenance set are expanded.
10. The resultant cell lines, containing an R4 integrase phage attachment site (attP), are referred to as PER.C6™ —R4attP cells.
   Testing the PER.C6™-R4attP Cell Line

A SARS or anthrax antibody is used to test and characterize the PER.C6™-R4attP cell line. Most of the SARS and anthrax antibodies are IgG1. The V_{H} and V_{L} variable regions of the antibodies are cloned and then assembled in a vector that contains IgG1 constant regions to produce full-length antibodies. The cDNAs for the heavy chain and the light chain can either be cloned into two separate donor plasmids or into a single donor plasmid in tandem driven by either two identical or two different promoters. An advantage of using a phage integrase is that there is no size limitation on the gene of interest. Both a two-plasmid system and a one-plasmid system will be used to express the full length antibodies.

The expression of monoclonal antibodies at research scale has been extensively described (Wurm et al., Nat Biotechnol 22, 1393-8 (2004); Andersen et al., Curr Opin Biotechnol 13, 117-23 (2002); Wirth et al., Gene 73, 419-26 (1988); Kim et al., Biotechnol Bioeng 58, 73-84 (1998); Gandor et al., FEBS Lett 377, 290-4 (1995); and Kito et al., Appl Microbiol Biotechnol 60, 442-8 (2002)), and also in PER.C6™ cells (Urlaub et al., Proc Natl Acad Sci USA 77, 4216-20 (1980)). These common procedures are followed with respect to the CHO—R4attP cell line. The serum-free medium and cell culture process is developed to optimize the antibody production for large-scale fermentation.

The expected production rate using the engineered PER.C6™-R4attP cell line will be about at least 30 pg/cell/day in serum-free medium. Once the cell line and the donor vector are developed, any antibody gene of interest can be conveniently cloned into the expression cassette of the donor vector (FIG. 2). Since selecting for high level expression clones only requires the screening of 30-50 colonies, a stable cell line that expresses high levels of an antibody can be rapidly generated in a cost-effective manner.

Characterization of the PER.C6™-R4attP Cell Line

The memorandum “Points to Consider in the Characterization of Cell Lines Used to Produce Biologicals (1993)” published by the Center for Biologics Evaluation and Research (CBER) of the FDA is followed to characterize the PER.C6™-R4attP cell line.

In addition, the R4 attP integration site is fully characterized, for example with regard to the number of copies and locus of the integration, by conventional methods, for example FISH, Southern blots, PCR, and DNA sequencing. Since the future integration of a gene of interest will be specifically targeted to the R4 attP site that has been previously engineered into the chromosome, characterization of the integration site of each individual gene of interest is trivial. Consequently, the future characterization of stable cell lines that express the gene of interest is significantly simplified, saving time and cost.

### Example 9

### Engineered PER.C6™ Cell Line for High Yield Protein Production with Enhanced Translation Using an IRES

The possibility and necessity of using an optimized IRES-element together with φC31 integrase to further increase the expression level is also tested. The optimized IRES-element is cloned into the donor vector, downstream of the promoter and upstream of the coding region for the gene of interest (FIG. 7). This IRES-element will significantly increase protein production by enhancing the translation efficiency of the target mRNA (Chappell et al., J Biol Chem 278, 33793-800 (2003); Owens et al., Proc Natl Acad Sci USA 98, 1471-6 (2001); and Chappell et al., (2000) Proc. Natl. Acad. Sci. U.S.A., 97, 1536-1541).

To obtain large quantities of therapeutic proteins and antibodies, overexpressing cell lines are developed that use novel translation-based technologies that are capable of much higher levels of protein production than is possible using traditional transcription based methods which increase the amount of target gene mRNA, e.g. through the use of strong promoters, chromosomal duplication, and selection of high expressing cell lines.

Translational enhancers have been developed recently using short RNA sequences that function as internal ribosome entry sites (IRESes) that recruit the translation machinery and facilitate translation initiation. Although the activity of individual IRES-elements is relatively weak, it was shown that IRES activity could be increased synergistically when particular IRES elements were linked together (Owens et al., Proc Natl Acad Sci U S A 98, 1471-6 (2001); and Chappell et al., (2000) Proc. Natl. Acad. Sci. U.S.A., 97, 1536-1541). In these studies, synthetic IRESes were tested in the intercistronic region of dicistronic mRNAs for their ability to enhance the translation of the second cistron. However, it was recently shown that one of these IRESes could also function as a potent translational enhancer when placed in the 5′ leader of a monocistronic mRNA. This synthetic IRES contained multiple linked copies of a 9-nt IRES-module from the 5′ leader of the Gtx homeodomain mRNA.

A goal is to identify IRES elements that function efficiently in PER.C6™ cells and use these individual elements to generate synthetic translational enhancers that function efficiently in PER.C6™ cells. Translational enhancers are also developed that function efficiently in human-hybrid and human cell lines that are used for large scale production.

Individual IRES elements that function efficiently in these cell lines are obtained using a selection methodology in which a cassette containing 18 random nucleotides is cloned into a selection vector and transfected into the cell line of interest (Owens et al., Proc Natl Acad Sci USA 98, 1471-6 (2001)). Selection experiments are performed using a GFP/CFP dicistronic retroviral vector. Cells containing active IRES elements are selected by FACS. Selected sequences are recovered and retested in a Renilla/Photinus (RPh) dual luciferase vector to show the IRES functions in another context and is not dependent on or influenced by sequences present in the GFP/CFP vectors used to select them. Various IRES elements are tested for their ability to synergize activity by linking together multiple copies of the same or different IRES-elements. Combinations of elements that show enhanced IRES activity are tested for their ability to function as translational enhancers in the 5′ leader of a monocistronic reporter RNA.

The synthetic translational enhancers that are generated are then tested in the 5′ leaders of mRNAs encoding therapeutic proteins or antibodies to determine which enhancer/gene combinations function most efficiently. Once particularly efficient combinations are identified, constructs are tested in scaled up culture conditions and further optimized if necessary to maximize antibody production.

### Example 10

### Engineered PER.C6™ Cell Line for High Yield Inducible Protein Production

Cell lines suitable for scale-up and manufacturing must have the combined capacity for fast growth and high specific-productivity. Due to the high expression level of the expression vector, the production cells might have difficulties growing when expressing high levels of foreign proteins, or the foreign proteins may aggregate during a prolonged growth phase. If this difficulty is encountered, an on-off switch is added to the donor vector to provide for inducible expression of the gene of interest in the PER.C6™ cell line. As such, the element would function to turn off the transgene expression during cell growth and would only turn on the expression when cells have grown to a critical amount and are ready for protein production. These switches are actuated by ligands that interact with an appropriate receptor system that conditionally interferes with or activates transcription. Several proprietary switches have been developed for gene therapy studies and can be used in the production system envisioned, including, but not limited to, the ARGENT system, the GENE SWITCH system, riboswitches, zinc finger proteins, ecdysone receptor-based systems, and the like. In addition, tetracycline-inducible and gas-inducible systems can also be utilized (Weber et al., Nat Biotechnol 22, 1440-4 (2004); and Weber et al., Metab Eng 7, 174-81 (2005)).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A site-specifically integrating target vector, said vector comprising:
(a) a first vector recombination site that recombines with a genomic recombination site in the presence of a first unidirectional site-specific recombinase;
(b) a second vector recombination site that recombines with a donor recombination site in the presence of a second unidirectional site-specific recombinase that is different from the first unidirectional site-specific recombinase;
(c) a first portion of a first selectable marker adjacent to the second vector recombination site's 3′ end; and
(d) a second selectable marker that is different from the first selectable marker.

2. The target vector of claim 1, wherein the genomic recombination site is a mammalian genomic recombination site.

3. The target vector of claim 1, wherein the first vector recombination site is a bacterial genomic recombination site (attB) or a phage genomic recombination site (attP).

4. The target vector of claim 1, wherein the first vector recombination site is a bacterial genomic recombination site (attB) and the genomic recombination site is a pseudo-phage genomic recombination site (pseudo-attP).

5. The target vector of claim 1, wherein the first vector recombination site is a phage genomic recombination site (attP) and the genomic recombination site is a pseudo-bacterial genomic recombination site (pseudo-attB).

6. The target vector of claim 1, wherein the first vector recombination site is a pseudo-bacterial genomic recombination site (pseudo-attB) or a pseudo-phage genomic recombination attP site (pseudo-attP).

7. The target vector of claim 1, wherein the second vector recombination site is a bacterial genomic recombination site (attB) or a phage genomic recombination site (attP).

8. The target vector of claim 1, wherein the second vector recombination site is a pseudo-bacterial genomic recombination site (pseudo-attB) or a pseudo-phage genomic recombination attP site (pseudo-attP).

9. The target vector of claim 1, wherein the first unidirectional site-specific recombinase is a φC31 phage recombinase, a TP901-1 phage recombinase, a R4 phage recombinase, a φFC1 phage recombinase, a φRv1 phage recombinase, or a φBT1 phage recombinase.

10. The target vector of claim 1, wherein the first unidirectional site-specific recombinase is a φC31 phage recombinase.

11. The target vector of claim 1, wherein the second unidirectional site-specific recombinase is a R4 phage recombinase.

12. A method of site-specifically integrating a polynucleotide encoding a protein of interest in a genome of a eukaryotic cell, said method comprising:
(a) introducing the target vector according to claim 1 into a mammalian cell comprising a first unidirectional site-specific recombinase and maintaining the mammalian cell under conditions sufficient for a recombination event mediated by the first unidirectional site-specific recombinase between the first vector recombination site and the genomic recombination site to site-specifically integrate the target vector into the genome of the mammalian cell;
(b) introducing a donor vector into the target cell comprising a second unidirectional site-specific recombinase, wherein the donor vector comprises the polynucleotide encoding a protein of interest and a donor recombination site, and maintaining the target cell under conditions sufficient for a recombination event mediated by the second unidirectional site-specific recombinase between the donor recombination site and the second vector recombination site of the target vector to site-specifically integrate the polynucleotide encoding a protein of interest in the genome of the mammalian cell;
wherein the first unidirectional site-specific recombinase is different from the second unidirectional site-specific recombinase.

13. The method of claim 12, further comprising selecting a cell that expresses the protein of interest.

14. The method of claim 12, wherein the first vector recombination site is a bacterial genomic recombination site (attB) or a phage genomic recombination site (attP).

15. The method of claim 12, wherein the first vector recombination site is a bacterial genomic recombination site (attB) and the genomic recombination site is a pseudo-phage genomic recombination site (pseudo-attP).

16. The method of claim 12, wherein the first vector recombination site is a phage genomic recombination site (attP) and the genomic recombination site is a pseudo-bacterial genomic recombination site (pseudo-attB).

17. The method of claim 12, wherein the first vector recombination site is a pseudo-bacterial genomic recombination site (pseudo-attB) or a pseudo-phage genomic recombination attP site (pseudo-attP).

18. The method of claim 12, wherein the second vector recombination site is a bacterial genomic recombination site (attB) or a phage genomic recombination site (attP).

19. The method of claim 12, wherein the second vector recombination site is a pseudo-bacterial genomic recombination site (pseudo-attB) or a pseudo-phage genomic recombination attP site (pseudo-attP).

20. The method of claim 12, wherein the donor recombination site is a bacterial genomic recombination site (attB) or a phage genomic recombination site (attP).

21. The method of claim 12, wherein the donor recombination site is a pseudo-bacterial genomic recombination site (pseudo-attB) or a pseudo-phage genomic recombination attP site (pseudo-attP).

22. The method of claim 12, wherein the first unidirectional site-specific recombinase is a φC31 phage recombinase, a TP901-1 phage recombinase, a R4 phage recombinase, a φFC1 phage recombinase, a φRv1 phage recombinase, or a φBT1 phage recombinase.

23. The method of claim 12, wherein the second unidirectional site-specific recombinase is a φC31 phage recombinase, a TP901-1 phage recombinase, a R4 phage recombinase, a φFC1 phage recombinase, a φRv1 phage recombinase, or a φBT1 phage recombinase.

24. The method of claim 12, wherein the first unidirectional site-specific recombinase is a φC31 phage recombinase.

25. The method of claim 12, wherein the second unidirectional site-specific recombinase is a R4 phage recombinase.

26. The method of claim 12, wherein the protein is a secreted protein.

27. The method of claim 12, wherein the secreted protein is an antibody.

28. The method of claim 12, wherein the cell is a mammalian cell.

29. The method of claim 28, wherein the mammalian cell is a rodent cell.

30. The method of claim 29, wherein the rodent cell is a CHO cell.

31. The method of claim 28, wherein the mammalian cell is a human cell.

32. The method of claim 31, wherein the human cell is a PER.C6™ cell.

33. An isolated eukaryotic cell, comprising:
a genomically integrated polynucleotide cassette comprising,
a first hybrid recombination site and a second hybrid recombination site flanking:
(a) a vector recombination site that recombines with a donor recombination site in the presence of a unidirectional site-specific recombinase;
(b) a first portion of a first selectable marker adjacent to the vector recombination site's 3′ end; and
(c) a second selectable marker that is different from the first selectable marker.

34. The isolated eukaryotic cell of claim 33, wherein the vector recombination site is a bacterial genomic recombination site (attB) or a phage genomic recombination site (attP).

35. The isolated eukaryotic cell of claim 33, wherein the donor recombination site is a bacterial genomic recombination site (attB) or a phage genomic recombination site (attP).

36. The isolated eukaryotic cell of claim 33, wherein the unidirectional site-specific recombinase is a φC31 phage recombinase, a TP901-1 phage recombinase, a R4 phage recombinase, a φFC1 phage recombinase, a φRv1 phage recombinase, or a φBT1 phage recombinase.

37. The isolated eukaryotic cell of claim 33, wherein the cell is a mammalian cell.

38. The isolated eukaryotic cell of claim 37, wherein the mammalian cell is a rodent cell.

39. The isolated eukaryotic cell of claim 38, wherein the rodent cell is a CHO cell.

40. The isolated eukaryotic cell of claim 37, wherein the mammalian cell is a human cell.

41. The isolated eukaryotic of claim 40, wherein the human cell is a PER.C6™ cell.

42. A kit for use in site-specifically integrating a polynucleotide into a genome of a cell in vitro, comprising:
(a) a vector according to claim 1; and
(b) a donor vector comprising:
(i) a multiple cloning site;
(ii) a donor recombination site; and
(iii) a second portion of a first selectable marker adjacent to the donor recombination site's 5′ end.

43. The kit of claim 42, further comprising a first unidirectional site-specific recombinase or nucleic acid encoding the same.

44. The kit of claim 43, further comprising a second unidirectional site-specific recombinase or nucleic acid encoding the same that is different from the first unidirectional site-specific recombinase.

45. The kit of claim 43, wherein the first unidirectional site-specific recombinase is a φC31 phage recombinase, a TP901-1 phage recombinase, a R4 phage recombinase, a φFC1 phage recombinase, a φRv1 phage recombinase, or a φBT1 phage recombinase.

46. The kit of claim 44, wherein the second unidirectional site-specific recombinase is a φC31 phage recombinase, a TP901-1 phage recombinase, a R4 phage recombinase, a φFC1 phage recombinase, a φRv1 phage recombinase, or a φBT1 phage recombinase.

47. A kit for use in producing a protein in a cell, comprising:
(a) an isolated eukaryotic cell according to claim 43; and
(b) a donor vector comprising:
(i) a multiple cloning site;
(ii) a donor recombination site; and
(iii) a second portion of a first selectable marker adjacent to the donor recombination site's 5′ end.

48. The kit of claim 47, further comprising a unidirectional site-specific recombinase or nucleic acid encoding the same.

49. The kit of claim 48, wherein the unidirectional site-specific recombinase is a φC31 phage recombinase, a TP901-1 phage recombinase, a R4 phage recombinase, a φFC1 phage recombinase, a φRv1 phage recombinase, or a φBT1 phage recombinase.


SEQUENCE LISTING (95 sequences)

SEQ ID NO: 1 - DNA, 18 bp, Artificial Sequence
  Oligonucleotide
cgtggggacgccgtacag

SEQ ID NO: 2 - DNA, 23 bp, Artificial Sequence
  Oligonucleotide
cccggtcaacatccagtacacct

SEQ ID NO: 3 - DNA, 37 bp, Artificial Sequence
  Oligonucleotide
aaaaaagaattcgtactgacggacacaccgaagcccc

SEQ ID NO: 4 - DNA, 49 bp, Artificial Sequence
  Oligonucleotide
cacggtaggcttgtactcggtcatggtggcgaccctacgcccccaactg

SEQ ID NO: 5 - DNA, 49 bp, Artificial Sequence
  Oligonucleotide
cagttgggggcgtagggtcgccaccatgaccgagtacaagcccacggtg

SEQ ID NO: 6 - DNA, 48 bp, Artificial Sequence
  Oligonucleotide
aaaaaacctttcgtcttcagacatgataagatacattgatgagtttgg

SEQ ID NO: 7 - DNA, 61 bp, Artificial Sequence
  Oligonucleotide
gatccaaaaaattaattaaaaaaaacaccggcgaaaaaagcgatcgcaaaaaaccagtgtg

SEQ ID NO: 8 - DNA, 53 bp, Artificial Sequence
  Oligonucleotide
ctggttttttgcgatcgcttttttcgccggtgttttttttaattaattttttg

SEQ ID NO: 9 - DNA, 24 bp, Artificial Sequence
  Oligonucleotide
gtcgacgaaataggtcacggtctc

SEQ ID NO: 10 - DNA, 24 bp, Artificial Sequence
  Oligonucleotide
tacgtcgacatgcccgccgtgacc

SEQ ID NO: 11 - DNA, 74 bp, Artificial Sequence
  Oligonucleotide
cgcgccaccatggcatgccctggcttcctgtgggcacttgtgatctccacctgcctcgagttttccatggctcg

SEQ ID NO: 12 - DNA, 70 bp, Artificial Sequence
  Oligonucleotide
ggtggtaccgtacgggaccgaaggacacccgtgaacactagaggtggacggagctcaaaaggtaccgagc

SEQ ID NO: 13 - DNA, 79 bp, Artificial Sequence
  Oligonucleotide
gatccgccaccatggcatgccctggcttcctgtgggcacttgtgatctccacgtgtcttgaattttccatggctttaat

SEQ ID NO: 14 - DNA, 73 bp, Artificial Sequence
  Oligonucleotide
gcggtggtaccgtacgggaccgaaggacacccgtgaacactagaggtgcacagaacttaaaaggtaccgaaat

SEQ ID NO: 15 - DNA, 55 bp, Artificial Sequence
  Oligonucleotide
aaaaaacacgtgtcttgaattttccatggctgaagtgcagctggtggagtctggg

SEQ ID NO: 16 - DNA, 38 bp, Artificial Sequence
  Oligonucleotide
aaaaaattaattaattatttacccggagacagggagag

SEQ ID NO: 17 - DNA, 48 bp, Artificial Sequence
  Oligonucleotide
aaaacctcgagttttccatggctgaaacgacactcacgcagtctccag

SEQ ID NO: 18 - DNA, 43 bp, Artificial Sequence
  Oligonucleotide
aaaaaagcggccgcttaacactctcccctgttgaagctctttg

SEQ ID NO: 19 - DNA, 22 bp, Artificial Sequence
  Oligonucleotide
gcttggtaccgagctcggatcc

SEQ ID NO: 20 - DNA, 24 bp, Artificial Sequence
  Oligonucleotide
gaagcttggtaccggtgaattcgg

SEQ ID NO: 21 - DNA, 47 bp, Artificial Sequence
  Oligonucleotide
cgaatcagcacggggtggcgcgccctgtggaatgtgtgtcagttagg

SEQ ID NO: 22 - DNA, 69 bp, Artificial Sequence
  Oligonucleotide
cgaatcagcacgaagtgcaccggtgtttaaacttaattaaagatctaaagccagcaaaagtcccatggt

SEQ ID NO: 23 - DNA, 41 bp, Artificial Sequence
  Oligonucleotide
aaaaaattaattaaaatgaaagaccccacctgtaggtttgg

SEQ ID NO: 24 - DNA, 62 bp, Artificial Sequence
  Oligonucleotide
aaaaaacaccggtgaaagtttaaacaaacctgcaggaatgaaagacccccgctgacgggtag

SEQ ID NO: 25 - DNA, 48 bp, Artificial Sequence
  Oligonucleotide
ttttttgaagacgaaaggctgtggaatgtgtgtcagttagggtgtgga

SEQ ID NO: 26 - DNA, 40 bp, Artificial Sequence
  Oligonucleotide
aaaaaacctgcaggaatgaaagacccccgctgacgggtag

SEQ ID NO: 27 - DNA, 141 bp, Artificial Sequence
  Oligonucleotide
gatccagcggaaacgagcgaaaaaaaaacagcggaaacgagcgaaaaaaaaacagcggaaacgagcgaaaaaaaaacagcggaaacgagcgaaaaaaaaacagcggaaacgagcggactcacaaccccagaaacagacatg

SEQ ID NO: 28 - DNA, 141 bp, Artificial Sequence
  Oligonucleotide
gatccatgtctgtttctggggttgtgagtccgctcgtttccgctgtttttttttcgctcgtttccgctgtttttttttcgctcgtttccgctgtttttttttcgctcgtttccgctgtttttttttcgctcgtttccgctg

SEQ ID NO: 29 - DNA, 143 bp, Artificial Sequence
  Oligonucleotide
cgcgccagcggaaacgagcgaaaaaaaaacagcggaaacgagcgaaaaaaaaacagcggaaacgagcgaaaaaaaaacagcggaaacgagcgaaaaaaaaacagcggaaacgagcggactcacaaccccagaaacagacatgg

SEQ ID NO: 30 - DNA, 143 bp, Artificial Sequence
  Oligonucleotide
cgcgccatgtctgtttctggggttgtgagtccgctcgtttccgctgtttttttttcgctcgtttccgctgtttttttttcgctcgtttccgctgtttttttttcgctcgtttccgctgtttttttttcgctcgtttccgctgg

SEQ ID NO: 31 - DNA, 42 bp, Artificial Sequence
  Oligonucleotide
aaaaaaaccctgcaggggcctccgcgccgggttttggcgcct

SEQ ID NO: 32 - DNA, 39 bp, Artificial Sequence
  Oligonucleotide
aaaaaaaacaccggtgcttatcggattttaccacatttg

SEQ ID NO: 33 - DNA, 42 bp, Artificial Sequence
  Oligonucleotide
aaaaaaaacaccggtgccgatatcgggtgccacgccgtcccg

SEQ ID NO: 34 - DNA, 35 bp, Artificial Sequence
  Oligonucleotide
aaaaaaaagcccgggcggcggcccgccagaaatcc

SEQ ID NO: 35 - DNA, 42 bp, Artificial Sequence; identical to SEQ ID NO: 31
  Oligonucleotide

SEQ ID NO: 36 - DNA, 35 bp, Artificial Sequence; identical to SEQ ID NO: 34
  Oligonucleotide

SEQ ID NO: 37 - DNA, 33 bp, Artificial Sequence
  Oligonucleotide
tacgaattcatcagccatatcacatttgtagag

SEQ ID NO: 38 - DNA, 29 bp, Artificial Sequence
  Oligonucleotide
ttatataccctctagagtctccgctcgga

SEQ ID NO: 39 - DNA, 100 bp, Artificial Sequence
  Oligonucleotide
ccgagcggagactctagagggtatataagcagagctcgtttagtgaaccgtcagatcgcctggagacgccatccacgctgttttgacctccatagaagac

SEQ ID NO: 40 - DNA, 93 bp, Artificial Sequence
  Oligonucleotide
aaaaaaggatccgagctcggtaccaagcttccaatgcaccgttcccggccgcggaggctggatcggtcccggtgtcttctatggaggtcaaaa

SEQ ID NO: 41 - DNA, 100 bp, Artificial Sequence; identical to SEQ ID NO: 39
  Oligonucleotide

SEQ ID NO: 42 - DNA, 98 bp, Artificial Sequence
  Oligonucleotide
aaaaaaggcgcgccgaattcaccggtaccaagcttccaatgcaccgttcccggccgcggaggctggatcggtcccggtgtcttctatggaggtcaaaa

SEQ ID NO: 43 - DNA, 33 bp, Artificial Sequence; identical to SEQ ID NO: 37
  Oligonucleotide

SEQ ID NO: 44 - DNA, 93 bp, Artificial Sequence; identical to SEQ ID NO: 40
  Oligonucleotide

SEQ ID NO: 45 - DNA, 33 bp, Artificial Sequence; identical to SEQ ID NO: 37
  Oligonucleotide

SEQ ID NO: 46 - DNA, 98 bp, Artificial Sequence; identical to SEQ ID NO: 42
  Oligonucleotide

SEQ ID NO: 47 - DNA, 42 bp, Artificial Sequence
  Oligonucleotide
gagagaggatccacgcgtctgtggaatgtgtgtcagttaggg

SEQ ID NO: 48 - DNA, 48 bp, Artificial Sequence
  Oligonucleotide
gagagagaattctctagacagacatgataagatacattgatgagtttg

SEQ ID NO: 49 - DNA, 48 bp, Artificial Sequence
  Oligonucleotide
ttttcactgcattcgacaattgtcatcccctcaggatatagtagtttc

SEQ ID NO: 50 - DNA, 45 bp, Artificial Sequence
  Oligonucleotide
gaccagcacgttgcccaggagttggaggtgcacaccaatgtggtg

SEQ ID NO: 51 - DNA, 45 bp, Artificial Sequence
  Oligonucleotide
caccacattggtgtgcacctccaactcctgggcaacgtgctggtc

SEQ ID NO: 52 - DNA, 43 bp, Artificial Sequence
  Oligonucleotide
gagagagctagcatttaaataaggacagggaagggagcagtgg

SEQ ID NO: 53 - DNA, 48 bp, Artificial Sequence; identical to SEQ ID NO: 49
  Oligonucleotide

SEQ ID NO: 54 - DNA, 43 bp, Artificial Sequence; identical to SEQ ID NO: 52
  Oligonucleotide

SEQ ID NO: 55 - DNA, 23 bp, Artificial Sequence
  Oligonucleotide
tgccccggggcttcacgttttcc

SEQ ID NO: 56 - DNA, 22 bp, Artificial Sequence
  Oligonucleotide
gcccgccgtgaccgtcgagaac

SEQ ID NO: 57 - DNA, 24 bp, Artificial Sequence
  Oligonucleotide
caggtcagaagcggttttcgggag

SEQ ID NO: 58 - DNA, 23 bp, Artificial Sequence
  Oligonucleotide
ccgctgacgctgccccgcgtatc

SEQ ID NO: 59 - DNA, 27 bp, Artificial Sequence
  Oligonucleotide
catctcaattagtcagcaaccatagtc

SEQ ID NO: 60 - DNA, 25 bp, Artificial Sequence
  Oligonucleotide
aagctctagctagaggtcgacggta

SEQ ID NO: 61 - DNA, 24 bp, Artificial Sequence
  Oligonucleotide
gtcgacgaaataggtcacggtctc

SEQ ID NO: 62 - DNA, 24 bp, Artificial Sequence
  Oligonucleotide
tacgtcgacatgcccgccgtgacc

SEQ ID NO: 63 - DNA, 24 bp, Artificial Sequence
  Oligonucleotide
caggtcagaagcggttttcgggag

SEQ ID NO: 64 - DNA, 23 bp, Artificial Sequence
  Oligonucleotide
tgccccggggcttcacgttttcc

SEQ ID NO: 65 - DNA, 22 bp, Artificial Sequence
  Oligonucleotide
gcccgccgtgaccgtcgagaac

SEQ ID NO: 66 - DNA, 23 bp, Artificial Sequence
  Oligonucleotide
ccgctgacgctgccccgcgtatc

SEQ ID NO: 67 - DNA, 14 bp, Artificial Sequence
  Oligonucleotide
gtcgacgatgtagg

SEQ ID NO: 68 - DNA, 73 bp, Artificial Sequence
  Oligonucleotide
gtactgacggacacaccgaagccccggcggcaaccctcagcggatgccccggggcttcacgttttcccaggtc

SEQ ID NO: 69 - DNA, 90 bp, Artificial Sequence
  Oligonucleotide
tcacggtctcgaagccgcggtgcgggtgccagggcgtgcccttgggctccccgggcgcgtactccacctcacccatatgatgaacgggtc

SEQ ID NO: 70 - DNA, 90 bp, Artificial Sequence
  Oligonucleotide
agaagcggttttcgggagtagtgccccaactggggtaacctttgagttctctcagttgggggcgtagggtcgccgacatgacacaagggg

SEQ ID NO: 71 - DNA, 34 bp, Artificial Sequence
  Oligonucleotide
gtgccagggcgtgcccttgggctccccgggcgcg

SEQ ID NO: 72 - DNA, 39 bp, Artificial Sequence
  Oligonucleotide
ccccaactggggtaacctttgagttctctcagttggggg

SEQ ID NO: 73 - DNA, 90 bp, Artificial Sequence
  Oligonucleotide
gaggtggcggtagttgatcccggcgaacgcgcggcgcaccgggaagccctcgccctcgaaaccgctgggcgcggtgctggtccatcgtca

SEQ ID NO: 74 - DNA, 91 bp, Artificial Sequence
  Oligonucleotide
cggtgagcacgggacgtgcgacggcgtcggcgggtgcggatacgcggggcagcgtcagcgggttctcgacggtcacggcgggcatgtcgac

SEQ ID NO: 75 - DNA, 58 bp, Artificial Sequence
  Oligonucleotide
ttgtgaccggggtggacacgtacgcgggtgcttacgaccgtcagtcgcgcgagcgcga

SEQ ID NO: 76 - DNA, 59 bp, Artificial Sequence
  Oligonucleotide
gtcgacgaaataggtcacggtctcgaagccgcggtgcgggtgccagggcgtgcccttgg

SEQ ID NO: 77 - DNA, 36 bp, Artificial Sequence
  Oligonucleotide
agttctctcagttgggggcgtagggtcgccaccatg

SEQ ID NO: 78 - DNA, 56 bp, Artificial Sequence
  Oligonucleotide
accgagtacaagcccacggtgcgcctcgccacccgcgacgacgtcccccgggccgt

SEQ ID NO: 79 - DNA, 26 bp, Artificial Sequence
  Oligonucleotide
cccatgaccatgccgaagcagtggta

SEQ ID NO: 80 - DNA, 24 bp, Artificial Sequence
  Oligonucleotide
cccatgaccatgctgggcaagatt

SEQ ID NO: 81 - DNA, 26 bp, Artificial Sequence
  Oligonucleotide
cccatgaccatgccgaagcagtggta

SEQ ID NO: 82 - DNA, 17 bp, Artificial Sequence
  Oligonucleotide
cccatgaccatgctgac

SEQ ID NO: 83 - DNA, 174 bp, Artificial Sequence
  Oligonucleotide
attccagaagtagtgaggaggcttttttggaggcctaggcttttgcaaaaagctcccgtcgacgaaataggtcacggtctcgaagccgcggtgcgggtgccagggcgtgcccttgagttctctcagttgggggcgtagggtcgccaccatgaccgagtacaagcccacggtgcg

SEQ ID NO: 84 - DNA, 174 bp, Artificial Sequence; identical to SEQ ID NO: 83
  Oligonucleotide

SEQ ID NO: 85 - DNA, 54 bp, Artificial Sequence
  Oligonucleotide
ccaactggggtaacctttgggctccccgggcgcgtactaattgcatgaagaatc

SEQ ID NO: 86 - DNA, 54 bp, Artificial Sequence; identical to SEQ ID NO: 85
  Oligonucleotide

SEQ ID NO: 87 - DNA, 174 bp, Artificial Sequence; identical to SEQ ID NO: 83
  Oligonucleotide

SEQ ID NO: 88 - DNA, 174 bp, Artificial Sequence; identical to SEQ ID NO: 83
  Oligonucleotide

SEQ ID NO: 89 - DNA, 61 bp, Artificial Sequence
  Oligonucleotide
gtagtgccccaactggggtaacctttgggctccccgggcgcgtactccacctcacccatct

SEQ ID NO: 90 - DNA, 61 bp, Artificial Sequence; identical to SEQ ID NO: 89
  Oligonucleotide

SEQ ID NO: 91 - DNA, 5437 bp, Artificial Sequence
  pR1 Plasmid
cctttcgtcttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcggtgggcctcgggggcgggtgcggggtcggcggggccgccccgggtggcttcggtcggagccatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcgaccctacgcccccaactgagagaactcaaaggttaccccagttggggcactactcccgaaaaccgcttctgacctgggaaaacgtgaagccccggggcaaagggcgaattctgcagataaattaggcaaaggaattcctcgacctgcagcccaagctaattcgcccttcgtggggacgccgtacagggacgtgcacctctcccgctgcaccgcctccagcgtcgccgccggctcgaaggacggggccgggatgacgatgcaggcggcgtgggaggtggcgcccaagttgcccatgaccatgccgaagcagtggtagaagggcaccggcagacacacccggtcctgctccgtgtagccgaccgtgcggcccacccagtagccgttgttgaggatgttgtggtgggagagcgtggcgcccttggggaagccggtggtgccggaggtgtactggatgttgaccgggaagggcgaattagcttggcactggcgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggacctgaccccacgcaacgcccaaaataataacccccacgaaccataaaccattccccatgggggaccccgtccctaacccacggggccagtggctatggcagggcctgccgccccgacgttggctgcgagccctgggccttcacccgaacttgggggttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgatccccacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatcaagctgatcttgcggcacgctgttgacgctgttaagcgggtcgctgcagggtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcggggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcgccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggc

SEQ ID NO: 92 - DNA, 8110 bp, Artificial Sequence
  pD1-DTX-1 Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagcttggtaccgagctcggatccgccaccatggcatgccctggcttcctgtgggcacttgtgatctccacgtgtcttgaattttccatggctgaagtgcagctggtggagtctgggggaggcttggtcaagcctggagggtccctgagactctcctgtgaagcctctggattcatcttcagtgactactacatgagctggatccgccaggctccagggaaggggctggaatggatttcatacattagtcctagtggtagtaccctatactacgcagactctatgaggggccgattcaccatctccagggacaacgccaagaactcactgtatctgcaaatgaacagcctgagagtcgaggacacggccgtgtatttctgtgcgagagagtaccccacaacttctaaagtcgctattaccccgaactggttcgacctctggggccagggaaccctggtcaccgtctcgagcgcgagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataattaattaaaaaaaacaccggcgaaaaaagcgatcgcaaaaaaccagtgtggtggaattctgcagataacgctagcgaattcaccggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgtcgacgaaataggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagaattgcatgaagaatctgcttagggttaggcgttttgcgctgcttcgctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagcttggtaccggtgaattcggcgcgccaccatggcatgccctggcttcctgtgggcacttgtgatctccacctgcctcgagttttccatggctgaaacgacactcacgcagtctccagccaccctgtctttgtctccaggggaaagagccaccctctcctgcagggccagtcagagtgttagcaccttcttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgaaacatctggccctctttcggcggagggaccaaagtggatatcaaacgtacggtggctgcaccatctgtattcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcggccgcaattcgctagcgttaacggatcgatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 93 - DNA, 7093 bp, Artificial Sequence
  pR1-DHFR Plasmid
cctttcgtcttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcggtgggcctcgggggcgggtgcggggtcggcggggccgccccgggtggcttcggtcggagccatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcgaccctacgcccccaactgagagaactcaaaggttaccccagttggggcactactcccgaaaaccgcttctgacctgggaaaacgtgaagccccggggcaaagggcgaattctgcagataaattaggcaaaggaattcctcgacctgcagcccaagctaattcgcccttcgtggggacgccgtacagggacgtgcacctctcccgctgcaccgcctccagcgtcgccgccggctcgaaggacggggccgggatgacgatgcaggcggcgtgggaggtggcgcccaagttgcccatgaccatgccgaagcagtggtagaagggcaccggcagacacacccggtcctgctccgtgtagccgaccgtgcggcccacccagtagccgttgttgaggatgttgtggtgggagagcgtggcgcccttggggaagccggtggtgccggaggtgtactggatgttgaccgggaagggcgaattagcttggcactggcgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggacctgaccccacgcaacgcccaaaataataacccccacgaaccataaaccattccccatgggggaccccgtccctaacccacggggccagtggctatggcagggcctgccgccccgacgttggctgcgagccctgggccttcacccgaacttgggggttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgatccccacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatcaagctgatcttgcggcacgctgttgacgctgttaagcgggtcgctgcagggtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagggcggcacctcgctaacggattcaccactccaagaagtgcaccggtgtttaattcgcccttaaaaaacaccggtgaaagtttaaacaaacctgcaggaatgaaagacccccgctgacgggtagtcaatcactcagaggagaccctcccaaggaacagcgagaccacaagtcggatgcaactgcaagagggtttattggatacacgggtacccgggcgactcagtcaatcggaggactggcgccccgagtgaggggttgtgggctcttttattgagctcggggagcagaagcgcgcgaacagaagcgagaagcgaactgattggttagttcaaataaggcacagggtcatttcaggtccttggggcaccctggaaacatctgatggttctctagaaactgctgagggctggaccgcatctggggaccatctgttcttggccctgagccggggcaggaactgcttaccacagatatcctgtttggcccatattcagctgttccatctgttcttggccctgagccggggcaggaactgcttaccacagatatcctgtttggcccatattcagctgttccatctgttcctgaccttgatctgaacttctctattctcagttatgtatttttccatgccttgcaaaatggcgttacttaagctagcttgccaaacctacaggtggggtctttcattttaattaagggcgaattaaacttaattaaagatctaaagccagcaaaagtcccatggtcttataaaaatgcatagctttaggaggggagcagagaacttgaaagcatcttcctgttagtctttcttctcgtagacttcaaacttatacttgatgcctttttcctcctggacctcagagaggacgcctgggtattctgggagaagtttatatttccccaaatcaatttctgggaaaaacgtgtcactttcaaattcctgcatgatccttgtcacaaagagtctgaggtggcctggttgattcatggcttcctggtaaacagaactgcctccgactatccaaaccatgtctactttacttgccaattccggttgttcaataagtcttaaggcatcatccaaacttttggcaagaaaatgagctcctcgtggtggttctttgagttctctactgagaactatattaattctgtcctttaaaggtcgattcttctcaggaatggagaaccaggttttcctacccataatcaccagattctgtttaccttccactgaagaggttgtggtcattctttggaagtacttgaactcgttcctgagcggaggccagggtaggtctccgttcttgccaatccccatattttgggacacggcgacgatgcagttcaatggtcgaaccatgatggcagcggggataaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagggcgcgcccccttggcagaacatatccatcgcgtccgccatctccagcagccgcacgcggcgcatctcggggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcgccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggc

SEQ ID NO: 94 - DNA, 9455 bp, Artificial Sequence
  pD1-DTX1-G418 Plasmid
gacggatcgggagatccacgcgtctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtctagagaattgcatgaagaatctgcttagggttaggcgttttgcgctgcttcgctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagcttggtaccgagctcggatccgccaccatggcatgccctggcttcctgtgggcacttgtgatctccacgtgtcttgaattttccatggctgaagtgcagctggtggagtctgggggaggcttggtcaagcctggagggtccctgagactctcctgtgaagcctctggattcatcttcagtgactactacatgagctggatccgccaggctccagggaaggggctggaatggatttcatacattagtcctagtggtagtaccctatactacgcagactctatgaggggccgattcaccatctccagggacaacgccaagaactcactgtatctgcaaatgaacagcctgagagtcgaggacacggccgtgtatttctgtgcgagagagtaccccacaacttctaaagtcgctattaccccgaactggttcgacctctggggccagggaaccctggtcaccgtctcgagcgcgagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataattaattaaaaaaaacaccggcgaaaaaagcgatcgcaaaaaaccagtgtggtggaattctgcagataacgctagcgaattcaccggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgtcgacgaaataggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagaattgcatgaagaatctgcttagggttaggcgttttgcgctgcttcgctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagcttggtaccggtgaattcggcgcgccaccatggcatgccctggcttcctgtgggcacttgtgatctccacctgcctcgagttttccatggctgaaacgacactcacgcagtctccagccaccctgtctttgtctccaggggaaagagccaccctctcctgcagggccagtcagagtgttagcaccttcttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgaaacatctggccctctttcggcggagggaccaaagtggatatcaaacgtacggtggctgcaccatctgtattcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcggccgcaattcgctagcgttaacggatcgatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 95 - DNA, 9753 bp, Artificial Sequence
  pD3-DTX1 Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagcttggtaccgagctcggatccgccaccatggcatgccctggcttcctgtgggcacttgtgatctccacgtgtcttgaattttccatggctgaagtgcagctggtggagtctgggggaggcttggtcaagcctggagggtccctgagactctcctgtgaagcctctggattcatcttcagtgactactacatgagctggatccgccaggctccagggaaggggctggaatggatttcatacattagtcctagtggtagtaccctatactacgcagactctatgaggggccgattcaccatctccagggacaacgccaagaactcactgtatctgcaaatgaacagcctgagagtcgaggacacggccgtgtatttctgtgcgagagagtaccccacaacttctaaagtcgctattaccccgaactggttcgacctctggggccagggaaccctggtcaccgtctcgagcgcgagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataattaattaaaaaaaacaccggcgaaaaaagcgatcgcaaaaaaccagtgtggtggaattctgcagataacgctagcgaattcaccggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttagagagagctagcatttaaataaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactccagcttggttcccaatagaccccgcaggccctacaggttgtcttcccaacttgccccttgctccataccacccccctccaccccataatattatagaaggacacctagtcagacaaaatgatgcaacttaattttattaggacaaggctggtgggcactggagtggcaacttccagggccaggagaggcactggggaggggtcacagggatgccacccgtagatctctcgagctattacacccactcgtgcaggctgcccaggggcttgcccaggctggtcagctgggcgatggcggtctcgtgctgctccacgaagccgccgtcctccacgtaggtcttctccaggcggtgctggatgaagtggtactcggggaagtccttcaccacgcccttgctcttcatcagggtgcgcatgtggcagctgtagaacttgccgctgttcaggcggtacaccaggatcacctggcccaccagcacgccgtcgttcatgtacaccacctcgaagctgggctgcaggccggtgatggtcttcttcatcacggggccgtcgttggggaagttgcggcccttgtactccacgcggtacacgaacatctcctcgatcaggttgatgtcgctgcggatctccaccaggccgccgtcctcgtagcgcagggtgcgctcgtacacgaagccggcggggaagctctggatgaagaagtcgctgatgtcctcggggtacttggtgaaggtgcggttgccgtactggaaggcggggctcaggatgtcgaaggcgaagggcaggggggcgcccttggtcacgcggatctgcaccagctggttgccgaacaggatgttgcccttgccgcagccctccatggtgaacacgtggttgttcaccacgccctccaggttcaccttgaagctcatgatctcctgcaggccggtgttcttcaggatctgcttgctcaccatggtgaattcaatcgatgttcgaatcccaattctttgccaaagtgatgggccagcacacagaccagcacgttgcccaggagttggaggtgcacaccaatgtggtgaatggtcaaatggcgtttgctgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacatttccccctccctatgcaaaagcgaaactactatatcctgaggggatgacaattgtcgaatgcataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgtcgacgaaataggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagaattgcatgaagaatctgcttagggttaggcgttttgcgctgcttcgctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagcttggtaccggtgaattcggcgcgccaccatggcatgccctggcttcctgtgggcacttgtgatctccacctgcctcgagttttccatggctgaaacgacactcacgcagtctccagccaccctgtctttgtctccaggggaaagagccaccctctcctgcagggccagtcagagtgttagcaccttcttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgaaacatctggccctctttcggcggagggaccaaagtggatatcaaacgtacggtggctgcaccatctgtattcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcggccgcaattcgctagcgttaacggatcgatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc